SEQUENCE LISTING (1 sequence)

SEQ ID NO: 1 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

(19) United States
(12) Patent Application Publication
(10) Pub. No.: US 2009/0087860 A1
(43) Pub. Date: Apr. 2, 2009

(54) HIGHLY SENSITIVE SYSTEM AND METHODS FOR ANALYSIS OF PROSTATE SPECIFIC ANTIGEN (PSA)

(76) Inventors: John A. Todd, Lafayette, CA (US); Ann Lu, Mountain View, CA (US)

Correspondence Address: WILSON SONSINI GOODRICH & ROSATI, 650 PAGE MILL ROAD, PALO ALTO, CA, 94304-1050, US

(21) Appl. No.: 12/197,159

(22) Filed: Aug. 22, 2008

Related U.S. Application Data
(60) Provisional application No. 60/957,808, filed on Aug. 24, 2007.
(60) Provisional application No. 61/062,210, filed on Jan. 23, 2008.

(51) Int. Cl. G01N 33/53; C07K 16/00

(52) U.S. Cl. 435/7.1; 530/391.3

(57) ABSTRACT

The invention described herein provides methods, compositions, kits, and systems for the sensitive detection of prostate specific antigen. Such methods, compositions, kits, and systems are useful in diagnosis, prognosis, and determination of methods of treatment in conditions that involve release of prostate specific antigen.

### CROSS-REFERENCE

This application claims the benefit of U.S. Provisional Application No. 60/957,808, fled Aug. 24, 2007 and U.S. Provisional Application 61/062,210 filed Jan. 23, 2008, which applications are incorporated herein by reference in their entirety.

### BACKGROUND OF THE INVENTION

Each year over 300,000 men are diagnosed with prostate cancer in the U.S. alone. It is the most prevalent form of cancer for men of all races. Both the incidence of prostate cancer and its associated mortality have been increasing over the past ten years. It is estimated that about 50-65% of the prostate cancer is localized, 9-17% has spread to an area near the prostate and 20-25% has metastasized to other parts of the body. Screening for prostate cancer is primarily by PSA (a blood test for Prostate Specific Antigen) and DRE (Digital Rectal Exam) testing. Confirmation of cancer is by biopsy. Treatment options depend on disease progression and include surgery, radiation and hormonal manipulation. Chemotherapy has not proven to be successful in the past, but is being tested in combination with other treatments.

Recently, it has been shown that women with breast cancer also exhibit PSA. Therefore, PSA can be used as a prognostic indicator. PSA production in breast tumors is associated with estrogen and/or progesterone receptor presence. Normally, PSA levels in female serum are undetectable in 90% of women.

### SUMMARY OF THE INVENTION

In one aspect the invention provides methods for detecting single molecules of prostate specific antigen (PSA). In some embodiments, the invention provides a method for determining the presence or absence of a single molecule of prostate specific antigen (PSA) or a fragment or complex thereof in a sample, including i) labeling the molecule, fragment, or complex, if present, with a label; and ii) detecting the presence or absence of the label, where the detection of the presence of the label indicates the presence of the single molecule, fragment, or complex of PSA in the sample. In some embodiments, the PSA is produced by the epithelial cells of the prostate gland. In some embodiments of the methods of the inventions, the level of PSA measured is the total amount of PSA. In some embodiments of the methods of the invention, the PSA is free PSA. In some embodiments of the methods of the invention, the PSA can be a PSA complex. In some embodiments the PSA complex is PSA-ACT. In some embodiments, the PSA complex is PSA-A2M. In some embodiments of the methods of the invention, a single molecule of prostate specific antigen can be detected at a limit of detection of less than about 100 pg/ml. In some embodiments of the methods of the invention, a single molecule or PSA can be detected at a level of detection of less than about 50 pg/ml. In some embodiments of the methods of the invention, a single molecule or PSA can be detected at a level of detection of less than about 10 pg/ml. In some embodiments of the methods of the invention, a single molecule or PSA can be detected at a level of detection of less than about 5 pg/ml. In some embodiments of the methods of the invention, a single molecule or PSA can be detected at a level of detection of less than about 1 pg/ml. In some embodiments of the methods of the invention, a single molecule or PSA can be detected at a level of detection of less than about 0.5 pg/ml. In some embodiments of the methods of the invention, a single molecule or PSA can be detected at a level of detection of less than about 0.1 pg/ml. In some embodiments, the PSA detected is PSA produced by the epithelial cells of the prostate gland. In some embodiments of the methods of the invention, the label includes a fluorescent moiety. In some embodiments, the fluorescent moiety is capable of emitting at least about 200 photons when simulated by a laser emitting light at the excitation wavelength of the moiety, where the laser is focused on a spot not less than about 5 microns in diameter that contains the moiety, and where the total energy directed at the spot by the laser is no more than about 3 microJoules. In some embodiments of the methods of the invention, the fluorescent moiety includes a molecule that contains at least one substituted indolium ring system in which the substituent on the 3-carbon of the indolium ring contains a chemically reactive group or a conjugated substance group. In some embodiments of the methods of the invention, the fluorescent moiety includes a dye. Examples of dyes include, but are not limited to, AlexaFluor 488, AlexaFluor 532, AlexaFluor 647, AlexaFluor 680 and AlexaFluor 700. In some embodiments of the methods of the invention, the fluorescent moiety includes AlexaFluor 647. In some embodiments, the fluorescent moiety includes a molecule that contains at least one substituted indolium ring system in which the substituent on the 3-carbon of the indolium ring contains a chemically reactive group or a conjugated substance group. In some embodiments of the methods of the invention, the label further includes a binding partner for the PSA molecule, fragment, or complex. In some embodiments of the methods of the invention, the binding partner includes an antibody specific to the PSA molecule, fragment, or complex. In some embodiments of the methods of the invention, the antibody can be a polyclonal antibody. In some embodiments of the methods of the invention, the antibody is a monoclonal antibody. In some embodiments of the methods of the invention, the methods further include capturing PSA or PSA complexes on a solid support. In some embodiments of the methods of the invention, the solid support can be a microtiter plate or paramagnetic beads. In some embodiments of the methods of the invention, the solid support includes a capture partner specific for the PSA or PSA complex that is attached to the solid support. In some embodiments of the methods of the invention, the attachment of the capture partner to the solid support is noncovalent. In some embodiments of the methods of the invention, the attachment of the capture partner to the solid support is covalent. In some embodiments of the methods of the invention, the covalent attachment of the capture partner is such that the capture partner is attached to the solid support in a specific orientation. In some embodiments of the methods of the invention, the specific orientation serves to maximize specific binding of the PSA or PSA complexes to the capture partner. In some embodiments of the methods of the invention, the capture partner comprises an antibody. In some embodiments of the methods of the invention, the antibody is a monoclonal antibody. In some embodiments of the methods of the invention, the sample is a blood, serum, or plasma sample. In some embodiments of the methods of the invention, the sample is a serum sample. In some embodiments of the methods of the invention, the label include a fluorescent moiety, and includes passing the label through a single molecule detector. In some embodiments of the methods of the invention, the single molecule detector include: a) an electromagnetic radiation source for stimulating the fluorescent moiety; b) a capillary flow cell for passing the fluorescent moiety; c) a source of motive force for moving the fluorescent moiety in the capillary flow cell; d) an interrogation space defined within the capillary flow cell for receiving electromagnetic radiation emitted from the electromagnetic source; e) an electromagnetic radiation detector operably connected to the interrogation space for measuring an electromagnetic characteristic of the stimulated fluorescent moiety; and f) a microscope objective lens situated between the interrogation space and the detector, where the lens is a high numerical aperture lens.

In some embodiments, the invention provides a method for prostate cancer wherein the PSA levels are measured post resection and where the levels of PSA post resection decrease to approximately less than 100 pg/ml. In some embodiments, the method provides for diagnosing the recurrence of prostate cancer after surgical resection. In some embodiments, the diagnosis of prostate cancer is made by comparing the level of PSA in a sample to a predetermined threshold level of PSA. In some embodiments, the predetermined threshold level of PSA is a low level of PSA. In some embodiments, the method for detecting recurrence of prostate cancer includes measuring a level of PSA in a sample less than 100 pg/ml. In some embodiments, the diagnosis of recurrence of prostate cancer is made by measuring the PSA levels from a series of samples. In some embodiments, the diagnosis of recurrence of prostate cancer is made by measuring the change in the level of PSA in a series of samples. In some embodiments the recurrence of prostate cancer is indicated by an increase in PSA levels from a predetermined threshold level of PSA. In some embodiments the diagnosis of recurrence of prostate cancer is not made as based on a finding of a decrease in PSA levels from a predetermined threshold level of PSA. In some embodiments, the level of PSA in a sample is measured, wherein the sample consists of blood, serum, plasma, broncheoalveolar lavage fluid, urine, cerebrospinal fluid, pleural fluid, synovial fluid, peritoneal fluid, amniotic fluid, gastric fluid, lymph fluid, intestinal fluid, tissue homogenate, cell extracts, saliva, sputum, stool, physiological secretions, tears, mucus, sweat, milk, semen, seminal fluid, vaginal secretions, fluid from ulcers, and other eruptions, blisters, and abcesses, and extracts of tissue including biopsies of normal malignant, and suspect tissues or any other constituents of the body which may contain PSA. In some embodiments, the sample is selected from blood or serum. In some embodiment, the invention herein provides for a method of monitoring decreases in a level of PSA after surgical resection, wherein the level of PSA in a sample obtained from an individual is measured wherein the sample is taken from the individual prior to surgical resectioning. A second sample is then taken from the individual after surgical resection is complete and the level of PSA in the second sample is compared to the level of PSA in the first sample. In some embodiments, what is provided herein is a method for monitoring the effectiveness of a therapeutic treatment in an individual comprising: i) measuring the level of PSA in a first sample obtained from the individual, wherein the first sample it taken from the individual prior to administration of a therapeutic treatment, and ii) measuring the level of PSA in a series of samples taken from the individual at different time points subsequent to beginning the therapeutic treatment, and iii) comparing the level of PSA prior to the therapeutic treatment to the level of PSA subsequent to the therapeutic treatment to determine the effectiveness of the therapeutic treatment. In a further embodiment, the therapeutic treatment is altered in response to the level of PSA measured in a sample subsequent to the administration of treatment.

In some embodiments, the invention disclosed herein provides for a method of diagnosing breast cancer in an individual comprising comparing the level of PSA in a sample obtained from the individual to a normal range of PSA levels wherein the normal range is determined from a distribution of PSA levels in a reference population consisting of normal individuals. In some embodiments, the invention provides a method of diagnosis for breast cancer wherein a level of PSA in a sample obtained from an individual is compared to a PSA sample in a normal range where the normal range is determined by determining the distribution of PSA in a reference normal population and then identifying the increase or decrease in PSA levels from the reference population. In some embodiments, the method further comprises detecting levels of PSA in a sample at a sensitivity of less than 100 pg/ml. In some embodiments, the method comprises identifying increases or decreases in levels of PSA from the reference population. In some embodiments, the reference population consists of a group of individuals selected based on a common trait. In a further embodiments, the trait is selected from at least one of race, age, stage of menopause, or other desired demographic. In some embodiments, the method is used to diagnose the stage of breast cancer in an individual by comparing the level of PSA in an individual to a range of PSA levels for different stages of breast cancer. In some embodiments, the PSA levels for different stages of breast cancer are determined from a distribution of PSA levels in a reference population consisting of individuals with a specific stage of breast cancer. In some embodiments, the level of PSA in a sample is measured, wherein the sample consists of blood, serum, plasma, broncheoalveolar lavage fluid, urine, cerebrospinal fluid, pleural fluid, synovial fluid, peritoneal fluid, amniotic fluid, gastric fluid, lymph fluid, intestinal fluid, tissue homogenate, cell extracts, saliva, sputum, stool, physiological secretions, tears, mucus, sweat, milk, semen, seminal fluid, vaginal secretions, fluid from ulcers, and other eruptions, blisters, and abcesses, and extracts of tissue including biopsies of normal malignant, and suspect tissues or any other constituents of the body which may contain PSA. In some embodiments, the sample is selected from blood or serum. In some embodiments, what is disclosed is a method for screening an individual for the presence of breast cancer comprising detecting a level of PSA in a series of samples taken from said individual and further detecting increases or decreases in PSA levels in the samples compared to a predetermined threshold level of PSA. In some embodiment, the level of PSA in a series of samples are detected at a level of sensitivity of less than 100 pg/ml.

In some embodiment, the method disclosed provides for a method for diagnosing a condition indicated by increased levels of kallikreins wherein the level of kallikreins in a sample is detected at a level of sensitivity of less than 100 pg/ml. In some embodiments, the kallikrein detected is human kallikrein 3 (hk3). In some embodiments, the method diagnosis a condition that affects at least one of the pancreas, salivary glands, colon, small intestine, esophagus, kidney, lymph node, prostate gland, stomach, thyroid, ureter, vagina, adrenal gland, skin, breast, brain, spinal cord, heart, tonsils, liver, cervix, fallopian tube, lung, thymus, trachea, testis, bone marrow, bone, and cartilage. In some embodiments, the condition affects the prostate gland. In some embodiments, the level of PSA in a sample is measured, wherein the sample consists of blood, serum, plasma, broncheoalveolar savage fluid, urine, cerebrospinal fluid, pleural fluid, synovial fluid, peritoneal fluid, amniotic fluid, gastric fluid, lymph fluid, intestinal fluid, tissue homogenate, cell extracts, saliva, sputum, stool, physiological secretions, tears, mucus, sweat, milk, semen, seminal fluid, vaginal secretions, fluid from ulcers, and other eruptions, blisters, and abcesses, and extracts of tissue including biopsies of normal malignant, and suspect tissues or any other constituents of the body which may contain PSA. In some embodiments, the sample is selected from seminal fluid or urine.

The invention provides a method for determining a diagnosis, prognosis, or method of treatment in an individual that includes: a) determining a concentration of PSA in a sample or determining the concentrations of PSA in a series of samples from the individual, where the concentration is determined by a PSA assay with a limit of detection for the PSA in the sample of less than about 100 pg/ml; and b) determining a diagnosis, prognosis, or method of treatment in the individual, based on the concentration in the sample, or on the concentrations in the series of samples. In some embodiments of the method of the invention, step b) comprises a diagnosis, prognosis, or method of treatment of an individual having, or susceptible to having, prostate cancer. In some embodiments of the method of the invention, step b) comprises a diagnosis, prognosis, or method of treatment for an individual in which recurrence of prostate cancer has occurred. In some embodiments of the method of the invention, step b) comprises a diagnosis, prognosis, or method of treatment of an individual having, or susceptible to having, breast cancer. In some embodiments of the method of the invention, step b) comprises a diagnosis, prognosis, or method of treatment for an individual in which recurrence of breast cancer has occurred. In some embodiments of the methods of the invention, step b) includes an analysis such as comparing the concentration or series of concentrations to a normal value for the concentration, comparing the concentration or series of concentrations to a predetermined threshold level, comparing the concentration or series of concentrations to a baseline value, and determining a rate of change of concentration for the series of concentrations. In some embodiments of the methods of the invention, step b) includes comparing the concentration of PSA in the sample with a predetermined threshold concentration, and determining a diagnosis, prognosis, or method of treatment if the sample concentration is greater than the threshold level. In some embodiments of the methods for detecting breast cancer of the invention, the threshold concentration is determined by determining the 99th percentile concentration of PSA in a group of normal individuals, and setting the threshold concentration at the 99th percentile concentration. In some embodiments of the methods of the invention, at least one sample is taken as a PSA blood test to measure the level of PSA in the blood. In some embodiments the sample is taken before or after a digital rectal exam. In some embodiments, the sample is taken before or after a mammogram. In some embodiments of the methods of the invention, the prostate specific antigen is selected from the group consisting of free prostate specific antigen. In some embodiments of the methods of the invention, the prostate specific antigen is complexed prostate specific antigen. In some embodiments of the methods of the invention, the concentration of prostate specific antigen is a concentration of total prostate specific antigen. In some embodiments of the methods of the invention, the concentration of prostate specific antigen is a concentration of a prostate specific antigen complex, prostate specific antigen fragment, or a combination thereof. In some embodiments of the methods of the invention, the concentration of free prostate specific antigen is compared to total prostate specific antigen. In some embodiments of the methods of the invention, the diagnosis, prognosis, or method of treatment is a diagnosis, prognosis, or method of treatment of myocardial infarct. In some embodiments of the methods of the invention, the concentration or series of concentrations is determined at or near the time the individual presents to a health professional with one or more symptoms indicative of prostate cancer or the possibility thereof. In some embodiments of the methods of the invention, the concentration or series of concentrations is determined at or near the time the individual presents to a health professional with one or more symptoms indicative of breast cancer or the possibility thereof. In some embodiments, the one or more symptoms of prostate cancer can be slowing or weakening of a urinary stream, increase in the frequency of urination, hematuria, or impotence, pain to the hips, spine, ribs, numbness in the legs or feet or loss of bladder or bowel control. In some embodiments, the one or more symptoms of breast cancer can be nipple tenderness, a lump or thickening in the breast or underarm area, change in the size or shape of the breast, inverted nipple, scaly, red or swollen skin of the breast, areola, or nipple, ridges or pitting in the skin of the breast, areola or nipple, or nipple discharge. In some embodiments, the concentration is determined by a method that includes detecting single molecules of free prostate specific antigen or complexes or fragments thereof. In some embodiments, the methods of the invention involve labeling prostate specific antigen or a prostate specific antigen complex with a label that comprises a fluorescent moiety. In some embodiments of the methods of the invention, the fluorescent moiety is capable of emitting at least about 200 photons when simulated by a laser emitting light at the excitation wavelength of the moiety, where the laser is focused on a spot 5 microns in diameter that contains the moiety, and where the total energy directed at the spot by the laser is no more than about 3 microJoules. In some embodiments of the methods of the invention, the fluorescent moiety includes a molecule that contains at least one substituted indolium ring system in which the substituent on the 3-carbon of the indolium ring contains a chemically reactive group or a conjugated substance group. In some embodiments of the methods of the invention, the fluorescent moiety includes a dye selected from the group consisting of AlexaFluor 488, AlexaFluor 532, AlexaFluor 647, AlexaFluor 680 or AlexaFluor 700. In some embodiments of the methods of the invention, the fluorescent moiety comprises AlexaFluor 647. In some embodiments of the methods of the invention, the label further comprises a binding partner for the prostate specific antigen. In some embodiments, the binding partner comprises an antibody specific to prostate specific antigen. In some embodiments, the antibody is a polyclonal antibody. In some embodiments of the methods of the invention, the methods further include capturing prostate specific antigen or prostate specific antigen complexes on a solid support. In some embodiments of the methods of the invention, the solid support can be a microtiter plate or paramagnetic beads. In some embodiments of the methods of the invention, the solid support includes a capture partner specific for the prostate specific antigen or prostate specific antigen complexes that is attached to the solid support. In some embodiments of the methods of the invention, the attachment of the capture partner to the solid support is noncovalent. In some embodiments of the methods of the invention, the attachment of the capture partner to the solid support is covalent. In some embodiments of the methods of the invention, the covalent attachment of the capture partner is such that the capture partner is attached to the solid support in a specific orientation. In some embodiments of the methods of the invention, the specific orientation serves to maximize specific binding of the prostate specific antigen or prostate specific antigen complex to the capture partner. In some embodiments of the methods of the invention, step i) further involves assessing another indicator for the individual, and step ii) involves determining a diagnosis, prognosis, or method of treatment in the individual, based on the concentration of prostate specific antigen and the assessment of the other indicator of the non-prostate specific antigen marker in the sample, or on the concentrations in the series of samples. In some embodiments, the other indicator is the concentration of one or more non-prostate specific antigen markers in the sample or the series of samples. In some embodiments of the methods of the invention, the one or more markers are markers of prostate cancer or breast cancer. In some embodiment, the one or more markers of prostate cancer can be prostate specific membrane antigen (PSMA), KIAA 18, KIAA 96, prostate carcinoma tumor antigen-1 (PCTA-1), prostate secretory protein (PSP), prostate acid phosphatase (PAP), human glandular kallekrein 2 (HK-2), prostate stem cell antigen (PSCA), PTI-1, CLAR1 (U.S. Pat. No. 6,361,948), PG1, BPC-1, prostate-specific transglutaminase, cytokeratin 15, semenogelin II, NAALADase, PD-41, p53, TCSF (U.S. Pat. No. 5,856,112), p300, actin, EGFR, and HER-2/neu protein. In some embodiments, the one or more markers of breast cancer can be estrogen, epidermal growth factor (EGF), transforming growth factor (TGF), prostaglandin E2 (PGE2); estrogen-regulated proteins such as pS2; interleukins (eg., IL-10); S-100 protein; vimentin; epithelial membrane antigen; bcl-2; CA15-3 (an aberrant form of polymorphic epithelial mucin (PEM)); CA 19-9; mucin core carbohydrates (eg., Tn antigen and Tn-like antigens); alpha-lactalbumin; lipid-associated sialic acid (LASA); galactose-N-acetylgalactosamine (Gal-GalNAC); GCDFP-15; Le(y)-related carbohydrate antigen; CA 125; urokinase-type plasminogen activator (uPA) and uPA related antigens and complexes (eg., LMW-uPA, HMW-upA, uPA aminoterminal fragment (ATF), uPA receptor (uPAR) and complexes with inhibitors such as PA1-1 and PA1-2); beta-glucuronidase; CD31; CD44 splice variants; blood group antigens. In some embodiments of the methods of the invention, the diagnosis, prognosis, or method of treatment is a diagnosis, prognosis, or method of treatment of a condition that is not cancer. In some embodiments, the condition is enlarged prostate.

In another aspect the invention includes compositions.

In some embodiments, the invention includes a composition for the detection of a prostate specific antigen molecule, fragment, or complex including a binding partner to prostate specific antigen molecule, fragment, or complex attached to a fluorescent moiety, where the fluorescent moiety is capable of emitting at least about 200 photons when simulated by a laser emitting light at the excitation wavelength of the moiety, where the laser is focused on a spot not less than about 5 microns in diameter that contains the moiety, and where the total energy directed at the spot by the laser is no more than about 3 microJoules. In some embodiments of the compositions of the invention, the binding partner comprises an antibody to the prostate specific antigen molecule, fragment, or complex. In some embodiments, the antibody is a polyclonal antibody. In some embodiments, the antibody is a monoclonal antibody. In some embodiments, the prostate specific antigen molecule, fragment, or complex is free prostate specific antigen. In some embodiments, the prostate specific antigen molecule, fragment, or complex is a prostate specific antigen complex. In some embodiments, the prostate specific antigen complex is PSA-ACT. In some embodiments, the prostate specific antigen complex is PSA-A2M. In some embodiments of the compositions of the invention, the fluorescent moiety comprises a molecule that comprises at least one substituted indolium ring system in which the substituent on the 3-carbon of the indolium ring contains a chemically reactive group or a conjugated substance group. In some embodiments, the fluorescent moiety includes a dye that can be AlexaFluor 488, AlexaFluor 532, AlexaFluor 647, AlexaFluor 680 or AlexaFluor 700. In some embodiments, the fluorescent moiety comprises AlexaFluor 647.

In some embodiments the invention involves a composition comprising a set of standards for the determination of a concentration of prostate specific antigen, where at least one of the standards is at a concentration of prostate specific antigen less than about 100 pg/ml.

In some embodiments the invention involves a kit containing a composition including an antibody to cardiac prostate specific antigen attached to a fluorescent dye moiety, where the moiety is capable of emitting at least about 200 photons when simulated by a laser emitting light at the excitation wavelength of the moiety, where the laser is focused on a spot not less than about 5 microns in diameter that contains the moiety, and where the total energy directed at the spot by the laser is no more than about 3 microJoules, where the composition is packaged in suitable packaging. In some embodiments of the kits of the invention, the prostate specific antigen is free prostate specific antigen or complexed prostate specific antigen. In some embodiments, the prostate specific antigen is PSA-ACT. In some embodiments, the prostate specific antigen is PSA-A2M. In some embodiments of the kits of the invention, the kits further include instructions. In some embodiments of the kits of the invention, the kits further include a composition containing a capture antibody. In some embodiments, the prostate specific antigen is prostate specific antigen attached to a solid support. In some embodiments, the solid support comprises a microtiter plate or paramagnetic microparticles. In some embodiments of the kits of the invention, the kits further include a component selected from the group consisting of wash buffer, assay buffer, elution buffer, and calibrator diluent. In some embodiments of the kits of the invention, the kit can further comprise a standard for the prostate specific antigen.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate schematic diagrams of the arrangement of the components of a single particle analyzer; FIG. 1A shows an analyzer that includes one electromagnetic source and one electromagnetic detector; FIG. 1B shows an analyzer that includes two electromagnetic sources and one electromagnetic detectors;

FIGS. 2A and 2B illustrate schematic diagrams of a capillary flow cell for a single particle analyzer; FIG. 2A shows the flow cell of an analyzer that includes one electromagnetic source; FIG. 2B shows the flow cell of an analyzer that includes two electromagnetic sources;

FIGS. 3A and 3B illustrate schematic diagrams showing conventional (A) and confocal (B) positioning of laser and detector optics of a single particle analyzer; FIG. 3A shows the arrangement for an analyzer that has one electromagnetic source and one electromagnetic detector; FIG. 3B shows the arrangement for an analyzer that has two electromagnetic sources and two electromagnetic detectors;

FIGS. 4A-4C illustrate comparison of different assay system for PSA detection; FIG. 4A shows the correlation of detection between Singulex PSA assay system and Centaur or ACCESS(R2=0.9916); FIG. 4B shows the correlation between Singulex PSA assay system and ACS:Centaur (R2=0.999); FIG. 4C shows the correlation between Singulex and Beckman ACCESS(R2=0.9832);

FIG. 5 is a graph showing the linearized standard curves for fours standards with level of detection less than 0.1 pg/ml. Four standard curves, represented by separate point on the graph show linearity and curve fit for all four standard curves up to 0.14 pg/ml of PSA (R2=0.9972);

FIG. 6 is a graph showing the linearized back interpolation of PSA standard curves with regression analysis for fit (R²=0.9947);

FIG. 7 is a graph showing the linear dilution of male plasma samples; male plasma samples were diluted 1:100, 1:1000, 1:10,000 but remained linear throughout; and

FIGS. 8A & 8B is a bar graph showing PSA distribution in 53 subjects with normal female sera and plasma; PSA concentrations were detected as low as about 0-0.1 pg/ml and as high as above 4 pg/ml.

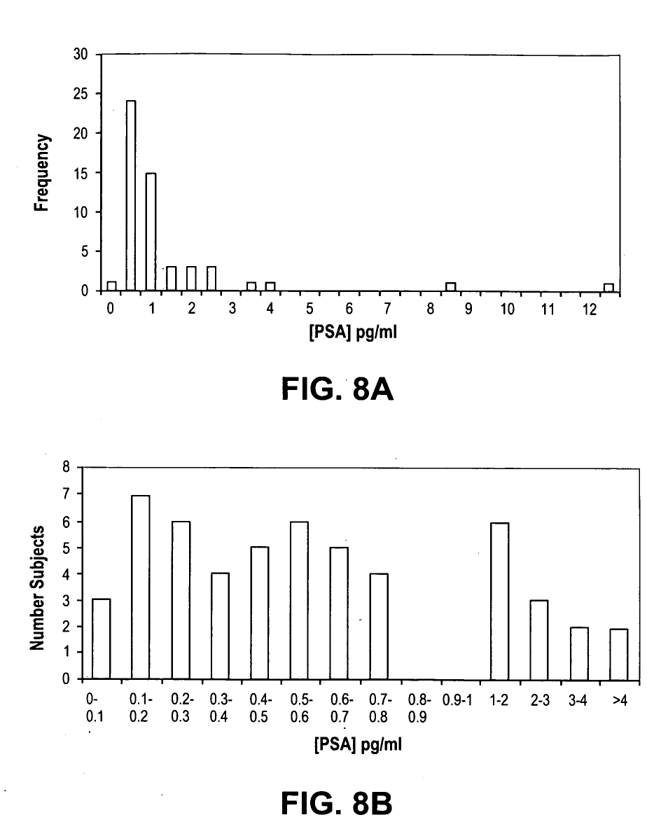

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### SUMMARY

### I. Introduction

### II. Prostate Specific Antigen

### III. Labels for Prostate Specific Antigen

A. Binding partners for prostate specific antigen
1. Antibodies
2. Cross-reacting antibodies

B. Fluorescent Moieties To Be Used With Binding Partners
1. Dyes
2. Quantum dots

C. Binding Partner-Fluorescent Moiety Compositions

### IV. Highly Sensitive Analysis of Prostate Specific Antigen

A. Sample

B. Sample preparation

C. Detection of prostate specific antigen and determination of concentration

### V. Instruments and Systems Suitable for Highly Sensitive Analysis of Prostate Specific Antigen

A. Apparatus/System

B. Single Particle Analyzer
1. Electromagnetic Radiation Source
2. Capillary Flow Cell
3. Motive Force
4. Detectors

C. Sampling System

D. Sample preparation system

E. Sample recovery

### VI. Methods Using Highly Sensitive Analysis of Prostate Specific Antigen

A. Samples

B. Determination of diagnosis, prognosis, or method of treatment
1. Prostate Cancer
2. Breast Cancer

C. Business Methods

### VII. Compositions

### VIII. Kits

### I. Introduction

The methods and compositions of the invention thus include methods and compositions for the highly sensitive detection and quantitation of prostate specific antigen, and compositions and methods for diagnosis, prognosis, and/or determination of treatment based on such highly sensitive detection and quantization.

The release into the blood of PSA is indicative of cancer, and provides the basis for their use as diagnostic or prognostic markers, or to aid in determination of treatment.

Prostate specific antigen (PSA) is most commonly known as a protein produced by the epithelial cells of the prostate gland. PSA is present in small quantities in the serum of normal men, and is often elevated in the presence of prostate cancer or other prostate disorders. Currently, a blood test is used to measure PSA levels as a method of early detection of prostate cancer. Higher than normal levels of PSA are associated with both localized and metastatic prostate cancer.

In addition to seminal fluid, the presence of PSA has been demonstrated in salivary glands, pancreas, breast (healthy breast tissues and breast tumors, breast cystic disease), various breast secretions (nipple aspirate fluid, milk of lactating women), periurethral gland, endometrial tissue, amniotic fluid, bronchoalveolar washing, ascitic fluid, plueral effusions, and cerebrospinal fluid. Very low levels of PSA are detectable in female sera. PSA has also been detected in a variety of tumors including, ovarian tumors, thyroid neoplasm, bile duct neoplasm, lung neoplasm, bladder neoplasm, sweat gland neoplasm, paraurethral gland neoplasm, salivary gland neoplasm, pancreas neoplasm, kidney, colon and liver neoplasm.

PSA is normally present in the blood at very low levels; normal PSA levels are defined as between zero (0) to four (4) ng per ml. Increased levels of PSA may suggest the presence of prostate cancer in men or breast or other cancers in women. Most PSA in the blood is bound to serum protein. A small amount of PSA is not bound to serum protein. PSA in this form is called free PSA.

PSA, used as a marker for cancer, especially cancer in women, or recurrence of cancer after surgical resection in men, is at a disadvantage in that there is a limit to its clinical usefulness. The best commercially available PSA assays claim an analytical limit of detection of 5-10 pg/ml and the lower limit of quantification of these assays is 50-100 pg/ml. A PSA assay could be used to detect recurrence of cancer tumors but the limited sensitivity of the current assays makes it difficult to determine if the PSA levels are from the tumor growing back.

### II. Prostate Specific Antigen (PSA)

Prostate-specific antigen (PSA), also known as also known as human kallikrein III (hk3), seminin, semenogelase, gamma-seminoprotein, and P-30, is a member of the human kallikrein gene family, 33 kDa chymotrypsin like protein that is synthesized exclusively by normal, hyperplastic, and malignant prostatic epithelia. Hence, the PSA's tissue-specific relationship has made it an excellent biomarker for identifying benign prostatic hyperplasia (BPH) and prostatic carcinoma (CaP) or metastatic cancer. Normal serum levels of PSA and blood are typically below 5 ng/ml, with elevated levels indicative of BPH or CaP. For example, serum levels of 200 ng/ml have been measured in end-stage metastatic CaP.

In blood, PSA is primarily found in three forms, as free-PSA, PSAα1-antichymotrysin complexes (PSA-ACT), and PSAα2-macroglobulin complexes (PSA-A2M). Trace amounts of PSA complexing to inter-trypsin inhibitor (IαI) and α1-protease inhibitor (α1PI) have also been reported. In some embodiments, the invention provides methods and compositions for the detection and/or determination of a concentration of total PSA, i.e. the sum of all or a substantial portion of the PSA in a sample, e.g., blood, serum or plasma sample, whether it is free or complexed. As used herein, “total PSA” refers to the total of all the three major forms of PSA. “Free PSA” refers to unbound PSA and is measured by antibodies that will normally recognize an epitope on PSA which PSA is not complexed, but will not recognize the same epitope if PSA is complexed.

It will be appreciated that an absolute total measurement need not be achieved, as long as a consistent proportion of the total is determined, which can be compared to standard values. It will also be appreciated that if a form of PSA is a minor constituent of the total, absence or low levels of detection of that form will not appreciably affect measures of total PSA. Currently, there are several measurements to be made of PSA in the blood or serum that indicate whether or not PSA levels have risen to problematic levels. In some cases, the amount of free PSA in the blood is measured. In other cases, the ratio of free PSA to total PSA is measured. In some cases, it has been suggested by some that the amount of PSA-ACT complex in the blood is a sufficient measure for indicating whether PSA levels have rises to an unhealthy level. Although the levels of PSA-A2M, IαI-PSA, and α1PI-PSA are currently too low to detect using current methods for detecting PSA, the method disclosed herein may be sensitive enough to measure the levels of PSA-A2M, IαI-PSA, and α1PI-PSA. As such, these PSA complexes may also be used as diagnostic indicators of whether PSA levels have risen to unhealthy levels.

### III. Labels for Prostate Specific Antigen (PSA)

In some embodiments, the invention provides methods and compositions that include labels for the highly sensitive detection and quantitation of prostate specific antigen.

One skilled in the art will recognize that many strategies can be used for labeling target molecules to enable their detection or discrimination in a mixture of particles. The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner to prostate specific antigen that is attached to a fluorescent moiety.

A. Binding Partners for PSA

Any suitable binding partner with the requisite specificity for prostate specific antigen to be detected may be used. Typically, binding partners bind to a region of PSA that is common to all or most of the different forms likely to be found in a sample. In some embodiments, a binding partner specific to one or more particular forms of PSA may be used, e.g., a binding partner to complexed PSA, free PSA, PSA-ACT complexes PSA-A2M complexes. Binding partners are known in the art and include, without limitation, aptamers, lectins, and receptors. The more specific the binding partner is to PSA the less likely the chance of a false measure of the level of PSA. For example, PSA has a similar amino acid sequence to human kallikrein 2 (hk2) and often antibodies meant to bind to PSA will instead bind to hk2 giving a false measure that PSA levels are elevated. Hence, it is in the best interest to use as specific a binding partner as necessary. In some embodiments, antibodies can be used as the binding partner.

1. Antibodies

In some embodiments, the binding partner is an antibody specific for prostate specific antigen (PSA). The term “antibody,” as used herein, is a broad term and is used in its ordinary sense, including, without limitation, to refer to naturally occurring antibodies as well as non-naturally occurring antibodies, including, for example purposes only, single chain antibodies, chimeric, bifunctional and humanized antibodies, as well as antigen-binding fragments thereof. In some embodiments, the antibody is specific for free PSA. In some embodiments, the antibody is specific for PSA complexes. In some embodiments, the label includes antibodies to any PSA. In some embodiments, an antibody specific to one or more particular forms of PSA may be used, e.g., a binding partner to complexed PSA, free PSA, total PSA, PSA-1-antichymotrypsin (ACT) complexes, PSA-2-macroglobulin (A2M) complexes, etc. Mixtures of antibodies are also encompassed by the invention, e.g., mixtures of antibodies to the various forms of the PSA (free, complexed, etc.), or mixtures of mixtures.

It will be appreciated that the choice epitope or region of PSA to which the antibody is raised will determine its specificity, e.g., for free PSA, for complexed PSA, and the like. In some embodiments, the antibody is specific to a specific amino acid region of PSA. The amino acid sequence for PSA (SEQ ID NO. 1) is shown in Table 1.

TABLE 1
SEQ ID NO:1 is the amino acid sequence of the matured form of PSA.

```
SEQUENCE LISTING
<160> NUMBER OF SEQ ID NOS:1
<200> SEQUENCE CHARACTERISTICS:
<210> SEQ ID NO 1
<211> LENGTH: 237
<212> TYPE: PRT
<213> ORGANISM: Homo sapiens
Ile Val Gly Gly Trp Glu Cys Glu Lys His Ser Gln Pro Trp Gln Val
1 5 10 15
Leu Val Ala Ser Arg Gly Arg Ala Val Cys Gly Gly Val Leu Val His
                        20 25 30
Pro Gln Trp Val Leu Thr Ala Ala His Cys Ile Arg Asn Lys Ser Val
                35 40 45
Ile Leu Leu Gly Arg His Ser Leu Phe His Pro Glu Asp Thr Gly Gln
        50 55 60
Val Phe Gln Val Ser His Ser Phe Pro His Pro Leu Tyr Asp Met Ser
65 70 75 80
Leu Leu Lys Asn Arg Phe Leu Arg Pro Gly Asp Asp Ser Ser His Asp
                                85 90 95
Leu Met Leu Leu Arg Leu Ser Glu Pro Ala Glu Leu Thr Asp Ala Val
                        100 105 110
Lys Val Met Asp Leu Pro Thr Gln Glu Pro Ala Leu Gly Thr Thr Cys
                115 120 125
Tyr Ala Ser Gly Trp Gly Ser Ile Glu Pro Glu Glu Phe Leu Thr Pro
        130 135 140
Lys Lys Leu Gln Cys Val Asp Leu His Val Ile Ser Asn Asp Val Cys
145 150 155 160
Ala Gln Val His Pro Gln Lys Val Thr Lys Phe Met Leu Cys Ala Gly
                                165 170 175
Arg Trp Thr Gly Gly Lys Ser Thr Cys Ser Gly Asp Ser Gly Gly Pro
                        180 185 190
Leu Val Cys Asn Gly Val Leu Gln Gly Ile Thr Ser Trp Gly Ser Glu
                195 200 205
Pro Cys Ala Leu Pro Glu Arg Pro Ser Leu Tyr Thr Lys Val Val His
        210 215 220
Tyr Arg Lys Trp Ile Lys Asp Thr Ile Val Ala Asn Pro
225 230 235
```

In some embodiments the antibody is a polyclonal antibody. Polyclonal antibodies are useful as binding partners. In some embodiments, the antibody is specific to amino acids 1-13 of human PSA. In some embodiments, the antibody is specific to the N-terminal peptide sequence of PSA. In some embodiments, the antibody is a polyclonal antibody specific to amino acids 53-64 of PSA. In some embodiments, the antibody is a polyclonal antibody specific to amino acids 80-91 of PSA. In some embodiments, the antibody is a polyclonal antibody specific to amino acids 151-164 of PSA.

Methods for producing antibodies are well established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). The antibodies used in the present methods may be obtained in accordance with known techniques, and may be monoclonal or polyclonal, and may be of any species of origin, including (for example) mouse, rat, rabbit, horse, or human, or may be chimeric antibodies. See, e.g., M. Walker et al., Molec. Immunol. 26:403 (1989). The antibodies may be recombinant monoclonal antibodies produced according to the methods disclosed in U.S. Pat. No. 4,474,893, or 4,816,567, and WO/1998/022509, which are herein incorporated by reference in their entirety. The antibodies may also be chemically constructed by specific antibodies made according to the method disclosed in U.S. Pat. Nos. 4,676,980 and 5,501,983, which is herein incorporated by reference in their entirety. Monoclonal and polyclonal antibodies to free and complexed PSA are also commercially available (Dako, Carpenteria, Calif., Scantibodies, Inc, Santee, Calif., BiosPacific, Emeryville, Calif.).

In some embodiments, the antibody is a mammalian, e.g., goat polyclonal anti-PSA, antibody. The antibody may be specific to specific regions of PSA. Capture binding partners and detection binding partner pairs, e.g., capture and detection antibody pairs, may be used in embodiments of the invention. Thus, in some embodiments, a heterogeneous assay protocol is used in which, typically, two binding partners, e.g., two antibodies, are used. One binding partner is a capture partner, usually immobilized on a solid support, and the other binding partner is a detection binding partner, typically with a detectable label attached. In some embodiments, the capture binding partner member of a pair is an antibody that is specific to all or substantially all forms of PSA. An example is an antibody, e.g., a monoclonal antibody, specific to free PSA, and PSA complexes. Thus, it is thought that the antibody binds to total PSA. Another example is a monoclonal antibody, specific to free PSA, but which does not cross-react with PSA-ACT. Such antibodies are available from BiosPacific, Emeryville, Calif. Another example is a monoclonal antibody, specific to PSA-ACT, which does not cross-react with PSA, ACT or CG-ACT. Other suitable antibody pairs know in the art can be used. Additionally, suitable antibody pairs can be designed.

2. Cross-Reacting Antibodies

In some embodiments it is useful to use an antibody that cross-reacts with a variety of species, either as a capture antibody, a detection antibody, or both. Such embodiments include the measurement of drug toxicity by determining, e.g., the release of PSA into the blood as a marker of cancer. A cross-reacting antibody allows studies of toxicity to be done in one species, e.g. a non-human species, and direct transfer of the results to studies or clinical observations of another species, e.g., humans, using the same antibody or antibody pair in the reagents of the assays, thus decreasing variability between assays. Thus, in some embodiments, one or more of the antibodies for use as a binding partner to the marker, e.g., PSA may be a cross-reacting antibody. In some embodiments, the antibody cross-reacts with the marker, e.g. PSA, from at least two species selected from the group consisting of human, monkey, dog, and rat. In some embodiments the antibody cross-reacts with the marker, e.g. PSA, from all members of the group consisting of human, monkey, dog, and rat.

B. Fluorescent Moieties to be Used with Binding Partners

In some embodiments, the binding partner, e.g., antibody, is attached to a fluorescent moiety. The fluorescence of the moiety will be sufficient to allow detection in a single molecule detector, such as the single molecule detectors described herein. A “fluorescent moiety,” as that term is used herein, includes one or more fluorescent entities whose total fluorescence is such that the moiety may be detected in the single molecule detectors described herein. Thus, a fluorescent moiety may comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired level of detection, accuracy, and precision of the assay. For example, in some embodiments, the fluorescence of the fluorescent moiety is such that it allows detection and/or quantitation of PSA at a level of detection of less than about 10, 5, 4, 3, 2, or 1 pg/ml and with a coefficient of variation of less than about 20, 15, 14, 13, 12, 11, 10, 9, 8, 7, 6, 5, 4, 3, 2, 1% or less, e.g., about 10% or less, in the instruments described herein. In some embodiments, the fluorescence of the fluorescent moiety is such that it allows detection and/or quantitation of PSA at a limit of detection of less than about 5 pg/ml and with a coefficient of variation of less than about 10%, in the instruments described herein. “Limit of detection,” as that term is used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

Furthermore, the moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the biomolecule of interest (e.g., protein) so that it may be analyzed using the analyzers and systems of the invention (e.g., does not cause precipitation of the protein of interest, or precipitation of a protein to which the moiety has been attached).

Fluorescent moieties, e.g., a single fluorescent dye molecule or a plurality of fluorescent dye molecules, that are useful in some embodiments of the invention may be defined in terms of their photon emission characteristics when stimulated by electromagnetic (EM) radiation. For example, in some embodiments, the invention utilizes a fluorescent dye moiety, e.g., a single fluorescent dye molecule or a plurality of fluorescent dye molecules, that is capable of emitting an average of at least about 10, 20, 30, 40, 50, 75, 100, 125, 150, 175, 200, 225, 250, 275, 300, 350, 400, 500, 600, 700, 800, 900, or 1000 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. It will be appreciated that the total energy may be achieved by many different combinations of power output of the laser and length of time of exposure of the dye moiety. For example, a laser of a power output of 1 mW may be used for 3 ms, 3 mW for 1 ms, 6 mW for 0.5 ms, 12 mW for 0.25 ms, and so on.

In some embodiments, the invention utilizes a fluorescent dye moiety, e.g., a single fluorescent dye molecule or a plurality of fluorescent dye molecules, that is capable of emitting an average of at least about 50 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. In some embodiments, the invention utilizes a fluorescent dye moiety, e.g., a single fluorescent dye molecule or a plurality of fluorescent dye molecules, that is capable of emitting an average of at least about 100 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. In some embodiments, the invention utilizes a fluorescent dye moiety, e.g., a single fluorescent dye molecule or a plurality of fluorescent dye molecules, that is capable of emitting an average of at least about 150 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. In some embodiments, the invention utilizes a fluorescent dye moiety, e.g., a single fluorescent dye molecule or a plurality of fluorescent dye molecules, that is capable of emitting an average of at least about 200 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. In some embodiments, the invention utilizes a fluorescent dye moiety, e.g., a single fluorescent dye molecule or a plurality of fluorescent dye molecules, that is capable of emitting an average of at least about 300 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. In some embodiments, the invention utilizes a fluorescent dye moiety e.g., a single fluorescent dye molecule or a plurality of fluorescent dye molecules, that is capable of emitting an average of at least about 500 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules.

In some embodiments, the fluorescent moiety comprises an average of at least about 1, 2, 3, 4, 5, 6, 7, 8, 9, or 10 fluorescent entities, e.g., fluorescent molecules. In some embodiments, the fluorescent moiety comprises an average of no more than about 2, 3, 4, 5, 6, 7, 8, 9, 10, or 11 fluorescent entities, e.g., fluorescent molecules. In some embodiments, the fluorescent moiety comprises an average of about 1 to about 11, or about 2 to about 10, or about 2 to about 8, or about 2 to about 6, or about 2 to about 5, or about 2 to about 4, or about 3 to about 10, or about 3 to about 8, or about 3 to about 6, or about 3 to about 5, or about 4 to about 10, or about 4 to about 8, or about 4 to about 6, or about 2, 3, 4, 5, 6, or more than about 6 fluorescent entities. In some embodiments, the fluorescent moiety comprises an average of about 2 to about 8 fluorescent moieties are attached. In some embodiments, an average of about 2 to about 6 fluorescent entities. In some embodiments, the fluorescent moiety comprises an average of about 2 to about 4 fluorescent entities. In some embodiments, the fluorescent moiety comprises an average of about 3 to about 10 fluorescent entities. In some embodiments, the fluorescent moiety comprises an average of about 3 to about 8 fluorescent entities. In some embodiments, the fluorescent moiety comprises an average of about 3 to about 6 fluorescent entities. What is meant by “average” is that, in a given sample that is a representative sample of a group of labels of the invention, where the sample contains a plurality of the binding partner-fluorescent moiety units, the molar ratio of the particular fluorescent entity of which the fluorescent moiety is comprise, to the binding partner, as determined by standard analytical methods, corresponds to the number or range of numbers specified. For example, in embodiments in which the label comprises a binding partner that is an antibody and a fluorescent moiety that comprises a plurality of fluorescent dye molecules of a specific absorbance, a spectrophometric assay may be used in which a solution of the label is diluted to an appropriate level and the absorbance at 280 nm is taken to determine the molarity of the protein (antibody) and an absorbance at, e.g., 650 nm (for AlexaFluor 647) is taken to determine the molarity of the fluorescent dye molecule. The ratio of the latter molarity to the former represents the average number of fluorescent entities (dye molecules) in the fluorescent moiety attached to each antibody.

1. Dyes

In some embodiments, the invention utilizes fluorescent moieties that comprise fluorescent dye molecules. In some embodiments, the invention utilizes a fluorescent dye molecule that is capable of emitting an average of at least about 50 photons when simulated by a laser emitting light at the excitation wavelength of the molecule, where the laser is focused on a spot of not less than about 5 microns in diameter that contains the molecule, and wherein the total energy directed at the spot by the laser is no more than about 3 microJoules. In some embodiments, the invention utilizes a fluorescent dye molecule that is capable of emitting an average of at least about 75 photons when simulated by a laser emitting light at the excitation wavelength of the molecule, where the laser is focused on a spot of not less than about 5 microns in diameter that contains the molecule, and wherein the total energy directed at the spot by the laser is no more than about 3 microJoules. In some embodiments, the invention utilizes a fluorescent dye molecule that is capable of emitting an average of at least about 100 photons when simulated by a laser emitting light at the excitation wavelength of the molecule, where the laser is focused on a spot of not less than about 5 microns in diameter that contains the molecule, and wherein the total energy directed at the spot by the laser is no more than about 3 microJoules. In some embodiments, the invention utilizes a fluorescent dye molecule that is capable of emitting an average of at least about 150 photons when simulated by a laser emitting light at the excitation wavelength of the molecule, where the laser is focused on a spot of not less than about 5 microns in diameter that contains the molecule, and wherein the total energy directed at the spot by the laser is no more than about 3 microJoules. In some embodiments, the invention utilizes a fluorescent dye molecule that is capable of emitting an average of at least about 200 photons when simulated by a laser emitting light at the excitation wavelength of the molecule, where the laser is focused on a spot of not less than about 5 microns in diameter that contains the molecule, and wherein the total energy directed at the spot by the laser is no more than about 3 microJoules

A non-inclusive list of useful fluorescent entities for use in the fluorescent moieties of the invention is given in Table 2, below. In some embodiments, the fluorescent entity is selected from the group consisting of Alexa Flour 488, 532, 647, 700, 750, Fluorescein, B-phycoerythrin, allophycocyanin, PBXL-3, and Qdot 605.

TABLE 2
FLUORESCENT ENTITIES

| Dye | Ex (nm) | E (M)−1 | Em (nm) | Mw |
|---|---|---|---|---|
| Bimane | 380 | 5,700 | 458 | 282.31 |
| Dapoxyl | 373 | 22,000 | 551 | 362.83 |
| Dimethylamino | 375 | 22,000 | 470 | 344.32 |
| coumarin-4-acetic acid | | | | |
| Marina blue | 365 | 19,000 | 460 | 367.26 |
| 8-Anilino naphthalene-1- | 372 | | 480 | |
| sulfonic acid | | | | |
| Cascade blue | 376 | 23,000 | 420 | 607.42 |
| Alexa Fluor 405 | 402 | 35,000 | 421 | 1028.26 |
| Cascade blue | 400 | 29,000 | 420 | 607.42 |
| Cascade yellow | 402 | 24,000 | 545 | 563.54 |
| Pacific blue | 410 | 46,000 | 455 | 339.21 |
| PyMPO | 415 | 26,000 | 570 | 582.41 |
| Alexa 430 | 433 | 15,000 | 539 | 701.75 |
| Atto-425 | 438 | | 486 | |
| NBD | 465 | 22,000 | 535 | 391.34 |
| Alexa 488 | 495 | 73,000 | 519 | 643.41 |
| Fluorescein | 494 | 79,000 | 518 | 376.32 |
| Oregon Green 488 | 496 | 76,000 | 524 | 509.38 |
| Atto 495 | 495 | | 522 | |
| Cy2 | 489 | 150,000 | 506 | 713.78 |
| DY-480-XL | 500 | 40,000 | 630 | 514.60 |
| DY-485-XL | 485 | 20,000 | 560 | 502.59 |
| DY-490-XL | 486 | 27,000 | 532 | 536.58 |
| DY-500-XL | 505 | 90,000 | 555 | 596.68 |
| DY-520-XL | 520 | 40,000 | 664 | 514.60 |
| Alexa Fluor 532 | 531 | 81,000 | 554 | 723.77 |
| BODIPY 530/550 | 534 | 77,000 | 554 | 513.31 |
| 6-HEX | 535 | 98,000 | 556 | 680.07 |
| 6-JOE | 522 | 75,000 | 550 | 602.34 |
| Rhodamine 6G | 525 | 108,000 | 555 | 555.59 |
| Atto-520 | 520 | | 542 | |
| Cy3B | 558 | 130,000 | 572 | 658.00 |
| Alexa Fluor 610 | 612 | 138,000 | 628 | |
| Alexa Fluor 633 | 632 | 159,000 | 647 | ca. 1200 |
| Alexa Fluor 647 | 650 | 250,000 | 668 | ca. 1250 |
| BODIPY 630/650 | 625 | 101,000 | 640 | 660.50 |
| Cy5 | 649 | 250,000 | 670 | 791.99 |
| Alexa Fluor 660 | 663 | 110,000 | 690 | |
| Alexa Fluor 680 | 679 | 184,000 | 702 | |
| Alexa Fluor 700 | 702 | 192,000 | 723 | |
| Alexa Fluor 750 | 749 | 240,000 | 782 | |
| B-phycoerythrin | 546, 565 | 2,410,000 | 575 | 240,000 |
| R-phycoerythrin | 480, 546, | 1,960,000 | 578 | 240,000 |
| | 565 | | | |
| Allophycocyanin | 650 | 700,000 | 660 | 700,000 |
| PBXL-1 | 545 | | 666 | |
| PBXL-3 | 614 | | 662 | |

Atto-tec dyes

| Name | Ex (nm) | Em (nm) | QY | (ns) |
|---|---|---|---|---|
| Atto 425 | 436 | 486 | 0.9 | 3.5 |
| Atto 495 | 495 | 522 | 0.45 | 2.4 |
| Atto 520 | 520 | 542 | 0.9 | 3.6 |
| Atto 560 | 561 | 585 | 0.92 | 3.4 |
| Atto 590 | 598 | 634 | 0.8 | 3.7 |
| Atto 610 | 605 | 630 | 0.7 | 3.3 |
| Atto 655 | 665 | 690 | 0.3 | 1.9 |
| Atto 680 | 680 | 702 | 0.3 | 1.8 |

Dyomics Fluors

| | | Molar absorbance* | Em | molecular weight# |
|---|---|---|---|---|
| label | Ex (nm) | [l · mol−1 · cm−1] | (nm) | [g · mol−1] |
| DY-495/5 | 495 | 70.000 | 520 | 489.47 |
| DY-495/6 | 495 | 70.000 | 520 | 489.47 |
| DY-495X/5 | 495 | 70.000 | 520 | 525.95 |
| DY-495X/6 | 495 | 70.000 | 520 | 525.95 |
| DY-505/5 | 505 | 85.000 | 530 | 485.49 |
| DY-505/6 | 505 | 85.000 | 530 | 485.49 |
| DY-505X/5 | 505 | 85.000 | 530 | 523.97 |
| DY-505X/6 | 505 | 85.000 | 530 | 523.97 |
| DY-550 | 553 | 122.000 | 578 | 667.76 |
| DY-555 | 555 | 100.000 | 580 | 636.18 |
| DY-610 | 609 | 81.000 | 629 | 667.75 |
| DY-615 | 621 | 200.000 | 641 | 578.73 |
| DY-630 | 636 | 200.000 | 657 | 634.84 |
| DY-631 | 637 | 185.000 | 658 | 736.88 |
| DY-633 | 637 | 180.000 | 657 | 751.92 |
| DY-635 | 647 | 175.000 | 671 | 658.86 |
| DY-636 | 645 | 190.000 | 671 | 760.91 |
| DY-650 | 653 | 170.000 | 674 | 686.92 |
| DY-651 | 653 | 160.000 | 678 | 888.96 |
| DYQ-660 | 660 | 117.000 | — | 668.86 |
| DYQ-661 | 661 | 116.000 | — | 770.90 |
| DY-675 | 674 | 110.000 | 699 | 706.91 |
| DY-676 | 674 | 145.000 | 699 | 807.95 |
| DY-680 | 690 | 125.000 | 709 | 634.84 |
| DY-681 | 691 | 125.000 | 708 | 736.88 |
| DY-700 | 702 | 96.000 | 723 | 668.86 |
| DY-701 | 706 | 115.000 | 731 | 770.90 |
| DY-730 | 734 | 185.000 | 750 | 660.88 |
| DY-731 | 736 | 225.000 | 759 | 762.92 |
| DY-750 | 747 | 240.000 | 776 | 712.96 |
| DY-751 | 751 | 220.000 | 779 | 814.99 |
| DY-776 | 771 | 147.000 | 801 | 834.98 |
| DY-780-OH | 770 | 70.000 | 810 | 757.34 |
| DY-780-P | 770 | 70.000 | 810 | 957.55 |
| DY-781 | 783 | 98.000 | 800 | 762.92 |
| DY-782 | 782 | 102.000 | 800 | 660.88 |
| EVOblue-10 | 651 | 101.440 | 664 | 389.88 |
| EVOblue-30 | 652 | 102.000 | 672 | 447.51 |

### Quantum Dots: Qdot 525, 565, 585, 605, 655, 705, 800

Suitable dyes for use in the invention include modified carbocyanine dyes. The modification of carbocyanine dyes includes the modification of an indolium ring of the carbocyanine dye to permit a reactive group or conjugated substance at the number 3 position. The modification of the indolium ring provides dye conjugates that are uniformly and substantially more fluorescent on proteins, nucleic acids and other biopolymers, than conjugates labeled with structurally similar carbocyanine dyes bound through the nitrogen atom at the number one position. In addition to having more intense fluorescence emission than structurally similar dyes at virtually identical wavelengths, and decreased artifacts in their absorption spectra upon conjugation to biopolymers, the modified carbocyanine dyes have greater photostability and higher absorbance (extinction coefficients) at the wavelengths of peak absorbance than the structurally similar dyes. Thus, the modified carbocyanine dyes result in greater sensitivity in assays that use the modified dyes and their conjugates. Preferred modified dyes include compounds that have at least one substituted indolium ring system in which the substituent on the 3-carbon of the indolium ring contains a chemically reactive group or a conjugated substance. Other dye compounds include compounds that incorporate an azabenzazolium ring moiety and at least one sulfonate moiety. The modified carbocyanine dyes that can be used to detect individual particles in various embodiments of the invention are described in U.S. Pat. No. 6,977,305, which is herein incorporated by reference in its entirety. Thus, in some embodiments the labels of the invention utilize a fluorescent dye that includes a substituted indolium ring system in which the substituent on the 3-carbon of the indolium ring contains a chemically reactive group or a conjugated substance group.

In some embodiments, the label comprises a fluorescent moiety that includes one or more Alexa dyes (Molecular Probes, Eugene, Oreg.). The Alexa dyes are disclosed in U.S. Pat. Nos. 6,977,305, 6,974,874, 6,130,101, and 6,974,305, which are herein incorporated by reference in their entirety. Some embodiments of the invention utilize a dye chosen from the group consisting of AlexaFluor 647, AlexaFluor 488, AlexaFluor 532, AlexaFluor 555, AlexaFluor 610, AlexaFluor 680, AlexaFluor 700, and AlexaFluor 750. Some embodiments of the invention utilize a dye chosen from the group consisting of AlexaFluor 488, AlexaFluor 532, AlexaFluor 647, AlexaFluor 700 and AlexaFluor 750. Some embodiments of the invention utilize the AlexaFluor 647 molecule, which has an absorption maximum between about 650 and 660 nm and an emission maximum between about 660 and 670 nm. The AlexaFluor 647 dye is used alone or in combination with other AlexaFluor dyes.

In addition, currently available organic fluors can be improved by rendering them less hydrophobic by adding hydrophilic groups such as polyethylene. Alternatively, currently sulfonated organic fluors such as the AlexaFluor 647 dye can be rendered less acidic by making them zwitterionic. Particles such as antibodies that are labeled with the modified fluors are less likely to bind non-specifically to surfaces and proteins in immunoassays, and thus enable assays that have greater sensitivity and lower backgrounds. Methods for modifying and improving the properties of fluorescent dyes for the purpose of increasing the sensitivity of a system that detects single particles are known in the art. Preferably, the modification improves the Stokes shift while maintaining a high quantum yield.

2. Quantum Dots

In some embodiments, the fluorescent label moiety that is used to detect a molecule in a sample using the analyzer systems of the invention is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which can be thought of the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One of the optical features of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dots dependent on the bandgap, it is therefore possible to control the output wavelength of a dot with extreme precision. In some embodiments the protein that is detected with the single particle analyzer system is labeled with a QD. In some embodiments, the single particle analyzer is used to detect a protein labeled with one QD and using a filter to allow for the detection of different proteins at different wavelengths.

QDs have broad excitation and narrow emission properties which when used with color filtering require only a single electromagnetic source for multiplex analysis of multiple targets in a single sample to resolve individual signals. Thus, in some embodiments, the analyzer system comprises one continuous wave laser and particles that are each labeled with one QD. Colloidally prepared QDs are free floating and can be attached to a variety of molecules via metal coordinating functional groups. These groups include but are not limited to thiol, amine, nitrile, phosphine, phosphine oxide, phosphonic acid, carboxylic acids or other ligands. By bonding appropriate molecules to the surface, the quantum dots can be dispersed or dissolved in nearly any solvent or incorporated into a variety of inorganic and organic films. Quantum dots (QDs) can be coupled to streptavidin directly through a maleimide ester coupling reaction or to antibodies through a meleimide-thiol coupling reaction. This yields a material with a biomolecule covalently attached on the surface, which produces conjugates with high specific activity. In some embodiments, the protein that is detected with the single particle analyzer is labeled with one quantum dot. In some embodiments the quantum dot is between about 10 and about 20 nm in diameter. In other embodiments, the quantum dot is between about 2 and about 10 nm in diameter. Useful Quantum Dots include QD 605, QD 610, QD 655, and QD 705. A particularly preferred Quantum Dot is QD 605.

C. Binding Partner-Fluorescent Moiety Compositions (Labels)

The labels of the invention generally contain a binding partner, e.g., antibody, bound to a fluorescent moiety to provide the requisite fluorescence for detection and quantitation in the instruments described herein. Any suitable combination of binding partner and fluorescent moiety for detection in the single molecule detectors described herein may be used as a label in the invention. In some embodiments, the invention provides a label for a PSA molecule, fragment, or complex, phosphorylated or oxidized form thereof, where the label includes an antibody to PSA and a fluorescent moiety. The antibody may be any suitable antibody as described above. In some embodiments, the antibody is an antibody to PSA. In some embodiments, the antibody is specific to a specific region of the PSA, e.g., specific to the promoter region of PSA. In some embodiments, the invention provides compositions comprising a fluorescent moiety attached to an anti-PSA antibody, e.g., a polyclonal antibody, such as a goat polyclonal antibody from those designated G126C available from BiosPacific, Emeryville. A fluorescent moiety may be attached such that the label is capable of emitting an average of at least about 50, 75, 100, 125, 150, 175, 200, 225, 250, 275, 300, 350, 400, 500, 600, 700, 800, 900, or 1000 photons when simulated by a laser emitting light at the excitation wavelength of the moiety, where the laser is focused on a spot of not less than about 5 microns in diameter that contains the label, and wherein the total energy directed at the spot by the laser is no more than about 3 microJoules. In some embodiments, the fluorescent moiety may be a fluorescent moiety that is capable of emitting an average of at least about 50, 100, 150, or 200 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. The fluorescent moiety may be a fluorescent moiety that includes one or more dye molecules with a structure that includes a substituted indolium ring system in which the substituent on the 3-carbon of the indolium ring contains a chemically reactive group or a conjugated substance group. The label composition may include a fluorescent moiety that includes one or more dye molecules selected from the group consisting of AlexaFluor 488, 532, 647, 700, or 750. The label composition may include a fluorescent moiety that includes one or more dye molecules selected from the group consisting of AlexaFluor 488, 532, 700, or 750. The label composition may include a fluorescent moiety that includes one or more dye molecules that are AlexaFluor 488. The label composition may include a fluorescent moiety that includes one or more dye molecules that are AlexaFluor 555. The label composition may include a fluorescent moiety that includes one or more dye molecules that are AlexaFluor 610. The label composition may include a fluorescent moiety that includes one or more dye molecules that are AlexaFluor 647. The label composition may include a fluorescent moiety that includes one or more dye molecules that are AlexaFluor 680. The label composition may include a fluorescent moiety that includes one or more dye molecules that are AlexaFluor 700. The label composition may include a fluorescent moiety that includes one or more dye molecules that are AlexaFluor 750.

In some embodiments the invention provides a composition for the detection of PSA that includes an AlexaFluor molecule, e.g., an AlexaFluor molecule selected from the described groups, such as an AlexaFluor 647 molecule attached to an antibody, e.g., a goat polyclonal anti-PSA antibody. In some embodiments the invention provides a composition for the detection of PSA that includes an average of about 1 to about 11, or about 2 to about 10, or about 2 to about 8, or about 2 to about 6, or about 2 to about 5, or about 2 to about 4, or about 3 to about 10, or about 3 to about 8, or about 3 to about 6, or about 3 to about 5, or about 4 to about 10, or about 4 to about 8, or about 4 to about 6, or about 2, 3, 4, 5, 6, or more than about 6 AlexaFluor 647 molecules molecule attached an antibody, e.g., a goat polyclonal anti-PSA antibody. In some embodiments the invention provides a composition for the detection of PSA that includes an average of about 1 to about 11, or about 2 to about 10, or about 2 to about 8, or about 2 to about 6, or about 2 to about 5, or about 2 to about 4, or about 3 to about 10, or about 3 to about 8, or about 3 to about 6, or about 3 to about 5, or about 4 to about 10, or about 4 to about 8, or about 4 to about 6, or about 2, 3, 4, 5, 6, or more than about 6 AlexaFluor 647 molecules molecule attached to an antibody, e.g., a goat polyclonal anti-PSA antibody. In some embodiments the invention provides a composition for the detection of PSA that includes an average of about 2 to about 10 AlexaFluor 647 molecules molecule attached to an antibody, e.g., a goat polyclonal anti-PSA antibody. In some embodiments the invention provides a composition for the detection of PSA that includes an average of about 2 to about 8 AlexaFluor 647 molecules molecule attached to an antibody, e.g., a goat polyclonal anti-PSA antibody. In some embodiments the invention provides a composition for the detection of PSA that includes an average of about 2 to about 6 AlexaFluor 647 molecules molecule attached to an antibody, e.g., a goat polyclonal anti-PSA antibody. In some embodiments the invention provides a composition for the detection of PSA that includes an average of about 2 to about 4 AlexaFluor 647 molecules molecule attached to an antibody, e.g., a goat polyclonal anti-PSA antibody. In some embodiments the invention provides a composition for the detection of PSA that includes an average of about 3 to about 8 AlexaFluor 647 molecules molecule attached to an antibody, e.g., a goat polyclonal anti-PSA antibody. In some embodiments the invention provides a composition for the detection of PSA that includes an average of about 3 to about 6 AlexaFluor 647 molecules molecule attached to an antibody, e.g., a goat polyclonal anti-PSA antibody. In some embodiments the invention provides a composition for the detection of PSA that includes an average of about 4 to about 8 AlexaFluor 647 molecules molecule attached to an antibody, e.g., a goat polyclonal anti-PSA antibody.

Attachment of the fluorescent moiety, or fluorescent entities that make up the fluorescent moiety, to the binding partner, e.g., antibody, may be by any suitable means; such methods are well-known in the art and exemplary methods are given in the Examples. In some embodiments, after attachment of the fluorescent moiety to the binding partner to form a label for use in the methods of the invention, and prior to the use of the label for labeling the protein of interest, it is useful to perform a filtration step. For example, an antibody-dye label may be filtered prior to use, e.g., through a 0.2 micron filter, or any suitable filter for removing aggregates. Other reagents for use in the assays of the invention may also be filtered, e.g., through a 0.2 micron filter, or any suitable filter. Without being bound by theory, it is thought that such filtration removes a portion of the aggregates of the, e.g., antibody-dye labels. As such, aggregates will bind as a unit to the protein of interest, but upon release in elution buffer, the aggregates are likely to disaggregate, which may cause false positives to result; i.e., several labels will be detected from an aggregate that has bound to only a single protein molecule of interest. Regardless of theory, filtration has been found to reduce false positives in the subsequent assay and to improve accuracy and precision.

### IV. Highly Sensitive Analysis of Prostate Specific Antigen

In one aspect the invention provides a method for determining the presence or absence of a single molecule of prostate specific antigen or a fragment or complex thereof in a sample, by i) labeling the molecule, fragment, or complex, if present, with a label; and ii) detecting the presence or absence of the label, where the detection of the presence of the label indicates the presence of the single molecule, fragment, or complex of PSA in the sample. As used herein, “molecule of PSA” includes a molecule that contains substantially the entire naturally-occurring amino acid sequence of the particular type of PSA, including post-translationally modified forms, e.g., phosphorylated forms, as well as oxidized or otherwise chemically altered forms. As used herein, a “fragment” of a molecule includes a molecule of PSA that contains less than the entire naturally-occurring amino acid sequence, including modifications as for the entire molecule. As used herein, a “complex” of a molecule of PSA includes a molecule of PSA or a fragment that is associated with one or more other molecules or substances, e.g., PSA associated with ACT, A2M, IαI, α1pI, and in some cases, PCI and PZP. In some embodiments, the method is capable of detecting the PSA at a limit of detection of less than about 150, 100, 80, 60, 40, 20, 10, 5, 1, 0.5, 0.1. or 0.05 pg/ml, e.g., less than about 100 pg/ml. In some embodiments, the method is capable of detecting the PSA at a limit of detection of less than about 150 pg/ml. In some embodiments, the method is capable of detecting PSA at a limit of detection of less than about 100 pg/ml. In some embodiments, the method is capable of detecting the PSA at a limit of detection of less than about 80 pg/ml. In some embodiments, the method is capable of detecting the PSA at a limit of detection of less than about 60 pg/ml. In some embodiments, the method is capable of detecting the PSA at a limit of detection of less than about 50 pg/ml. In some embodiments, the method is capable of detecting the PSA at a limit of detection of less than about 25 pg/ml. In some embodiments, the method is capable of detecting the PSA at a limit of detection of less than about 10 pg/ml. In some embodiments, the method is capable of detecting the PSA at a limit of detection of less than about 5 pg/ml. In some embodiments, the method is capable of detecting the PSA at a limit of detection of less than about 1 pg/ml. In some embodiments, the method is capable of detecting the PSA at a limit of detection of less than about 0.5 pg/ml. In some embodiments, the method is capable of detecting the PSA at a limit of detection of less than about 0.1 pg/ml. In some embodiments, the method is capable of detecting the PSA at a limit of detection of less than about 0.05 pg/ml. Detection limits may be determined by use of the appropriate National Institute of Standards and Technology reference standard material.

The methods provided herein also include methods of determining a concentration of PSA in a sample by detecting single molecules of PSA in the sample. The “detecting” of a single molecule of PSA includes detecting the molecule directly or indirectly. In the case of indirect detection, labels that corresponds to single molecules of PSA, e.g., a labels that have been attached to the single molecules of PSA, may be detected.

Types of PSA for detection are as described herein, e.g., free PSA, PSA-ACT, PSA-A2M, PSA-IαI, or PSA-α1PI. In some embodiments, total PSA is detected and/or quantitated. In some embodiments, free PSA is detected and/or quantified. In some embodiments, a PSA complex is detected and/or quantitated.

A. Sample

The sample used to detect the level of PSA may be any suitable sample. Typically, the sample is a biological sample, e.g., a biological fluid. Such fluids include, without limitation, exhaled breath condensate (EBC), bronchoalveolar lavage fluid (BAL), blood, serum, plasma, urine, cerebrospinal fluid, pleural fluid, synovial fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the target particle of interest. Other similar specimens such as cell or tissue culture or culture broth are also of interest.

In some embodiments, the sample is a blood sample. In some embodiments the sample is a plasma sample. In some embodiments the sample is a serum sample.

B. Sample Preparation

In general, any method of sample preparation may be used that produces a label corresponding to a molecule of cardiac troponin that is wished to be measured, where the label is detectable in the instruments described herein. As is known in the art, sample preparation in which a label is added to one or more particles may be performed in a homogeneous or heterogeneous format. In some embodiments, the sample preparation is formed in a homogenous format. For analyzer systems employing a homogenous format, unbound label is not removed from the sample. See, e.g., U.S. patent application Ser. No. 11/048,660, incorporated by reference herein in its entirety. In some embodiments, the particle or particles of interest are labeled by addition of labeled antibody or antibodies that bind to the particle or particles of interest.

In some embodiments, a heterogeneous assay format is used where, typically, a step is employed for removing unbound label. Such assay formats are well-known in the art. One particularly useful assay format is a sandwich assay, e.g., a sandwich immunoassay. In this format, the molecule of interest, e.g., marker of a biological state, is captured, e.g., on a solid support, using a capture binding partner. Unwanted molecules and other substances may then optionally be washed away, followed by binding of a label comprising a detection binding partner and a detectable label, e.g., fluorescent moiety. Further washes remove unbound label, then the detectable label is released, usually though not necessarily still attached to the detection binding partner. In alternative embodiments, sample and label are added to the capture binding partner without a wash in between, e.g., at the same time. Other variations will be apparent to one of skill in the art.

In some embodiments, the method for detecting PSA uses a sandwich assay with antibodies, e.g., polyclonal antibodies, as capture binding partners. The method comprises binding PSA molecules in a sample to a capture antibody that is immobilized on a binding surface, and binding the detection antibody to the PSA molecule to form a “sandwich” complex. The detection antibody comprises a detectable fluorescent label, as described herein, which is detected, e.g., using the single molecule analyzers of the invention. Both the capture and detection antibodies specifically bind PSA. Many example of sandwich immunoassays are known, and some are described in U.S. Pat. Nos. 4,168,146 and 4,366,241, both of which are incorporated herein by reference. Further examples specific to PSA are described in the Examples.

The capture binding partner may be attached to a solid support, e.g., a microtiter plate or paramagnetic beads. In some embodiments, the invention provides a binding partner for PSA attached to a paramagnetic bead. Any suitable binding partner that is specific for PSA or PSA complexes may be used. The binding partner may be an antibody, e.g., a polyclonal antibody. The antibody may be specific for free PSA or for complexed PSA, or specific to all or substantially all forms of PSA (total PSA or PSA complexes), likely to be found in the sample of interest. Production and sources of antibodies to PSA are described elsewhere herein. Preferred antibodies for measuring free PSA are those that not substantially affected by PSA complex formation and which do not cross-react with ACT or CG-ACT. In some embodiments, the antibody is specific for a specific region of a PSA. In some embodiments, the region includes amino acids 1-13 of PSA. In some embodiments, the region includes amino acids 87-91 of PSA. Such antibodies are well-known in the art and are available from, e.g. BiosPacific, Emeryville, Calif. An example of a capture antibody useful in embodiments of the invention is an antibody, e.g., a polyclonal antibody, that reacts with free PSA and PSA forming complexes with other components. An exemplary antibody of this type is Polyclonal Antibody Clone Number G126C, available from BiosPacific, Emeryville, Calif. It will be appreciated that antibodies identified herein as useful as a capture antibody may also be useful as detection antibodies, and vice versa.

The attachment of the binding partner, e.g., antibody, to the solid support may be covalent or noncovalent. In some embodiments, the attachment is noncovalent. An example of a noncovalent attachment well-known in the art is biotin-avidin/streptavidin interactions. Thus, in some embodiments, a solid support, e.g., a microtiter plate or a paramagnetic bead, is attached to the capture binding partner, e.g., antibody, through noncovalent attachment, e.g., biotin-avidin/streptavidin interactions. In some embodiments, the attachment is covalent. Thus, in some embodiments, a solid support, e.g., a microtiter plate or a paramagnetic bead, is attached to the capture binding partner, e.g., antibody, through covalent attachment. Covalent attachment in which the orientation of the capture antibody is such that capture of the molecule of interest is optimized is especially useful. For example, in some embodiments, a solid support, e.g., a microtiter plate or a paramagnetic microparticle, may be used in which the attachment of the binding partner, e.g., antibody, is an oriented attachment, e.g., a covalent oriented attachment.

An exemplary protocol for oriented attachment of an antibody to a solid support is as follows: IgG is dissolved in 0.1M sodium acetate buffer, pH 5.5 to a final concentration of 1 mg/ml. An equal volume of ice-cold 20 mM sodium periodate in 0.1M sodium acetate, pH 5.5 is added. The IgG is allowed to oxidize for ½ hour on ice. Excess periodate reagent is quenched by the addition of 0.15 volume of 1M glycerol. Low molecular weight byproducts of the oxidation reaction are removed by ultrafiltration. The oxidized IgG fraction is diluted to a suitable concentration (typically 0.5 micrograms IgG per ml) and reacted with hydrazide-activated multiwell plates for at least two hours at room temperature. Unbound IgG is removed by washing the multiwell plate with borate buffered saline or another suitable buffer. The plate may be dried for storage, if desired. A similar protocol may be followed for microbeads if the material of the microbead is suitable for such attachment.

In some embodiments, the solid support is a microtiter plate. In some embodiments, the solid support is a paramagnetic bead. An exemplary paramagnetic bead is Streptavidin C1 (Dynal, 650.01-03). Other suitable beads will be apparent to those of skill in the art. Methods for attachment of antibodies to paramagnetic beads are well-known in the art. One example is given in the Examples.

The prostate specific antigen is contacted with the capture binding partner, e.g., capture antibody immobilized on a solid support. Some sample preparation may be used; e.g., preparation of serum from blood samples or concentration procedures before the sample is contacted with the capture antibody. Protocols for binding of proteins in immunoassays are well-known in the art and are included in the Examples.

C. Detection of Prostate Specific Antigen And Determination Of Concentration

Following elution, the label is run through a single molecule detector in, e.g., the elution buffer. A processing sample may contain no label, a single label, or a plurality of labels. The number of labels corresponds or is proportional to (if dilutions or fractions of samples are used) the number of molecules of prostate specific antigen captured during the capture step.

Any suitable single molecule detector capable of detecting the label used with the protein of interest may be used. Suitable single molecule detectors are described herein. Typically the detector will be part of a system that includes an automatic sampler for sampling prepared samples, and, optionally, a recovery system to recover samples.

In some embodiments, the processing sample is analyzed in a single molecule analyzer that utilizes a capillary flow system, and that includes a capillary flow cell, a laser to illuminate an interrogation space in the capillary through which processing sample is passed, a detector to detect radiation emitted from the interrogation space, and a source of motive force to move the processing sample through the interrogation space. In some embodiments, the single molecule analyzer further comprises a microscope objective lens that collects light emitted from the processing sample as it passes through the interrogation space, e.g., a high numerical aperture microscope objective. In some embodiments, the laser and detector are in a confocal arrangement. In some embodiments, the laser is a continuous wave laser. In some embodiments, the detector is an avalanche photodiode detector. In some embodiments, the source of motive force is a pump to provide pressure. In some embodiments, the invention provides an analyzer system that includes a sampling system capable of automatically sampling a plurality of samples providing a fluid communication between a sample container and the interrogation space. In some embodiments, the interrogation space has a volume of between about 0.001 and about 500 pL, or between about 0.01 pL and about 100 pL, or between about 0.01 pL and about 10 pL, or between about 0.01 pL and about 5 pL, or between about 0.01 pL and about 0.5 pL, or between about 0.02 pL and about 300 pL, or between about 0.02 pL and about 50 pL or between about 0.02 pL and about 5 pL or between about 0.02 pL and about 0.5 pL or between about 0.02 pL and about 2 pL, or between about 0.05 pL and about 50 pL, or between about 0.05 pL and about 5 pL, or between about 0.05 pL and about 0.5 pL, or between about 0.05 pL and about 0.2 pL, or between about 0.1 pL and about 25 pL. In some embodiments, the interrogation space has a volume between about 0.004 pL and about 100 pL. In some embodiments, the interrogation space has a volume between about 0.02 pL and about 50 pL. In some embodiments, the interrogation space has a volume between about 0.001 pL and about 10 pL. In some embodiments, the interrogation space has a volume between about 0.001 pL and about 10 pL. In some embodiments, the interrogation space has a volume between about 0.01 pL and about 5 pL. In some embodiments, the interrogation space has a volume between about 0.02 pL and about 5 pL. In some embodiments, the interrogation space has a volume between about 0.05 pL and about 5 pL. In some embodiments, the interrogation space has a volume between about 0.05 pL and about 10 pL. In some embodiments, the interrogation space has a volume between about 0.5 pL and about 5 pL. In some embodiments, the interrogation space has a volume between about 0.02 pL and about 0.5 pL.

In some embodiments, the single molecule detector used in the methods of the invention utilizes a capillary flow system, and includes a capillary flow cell, a continuous wave laser to illuminate an interrogation space in the capillary through which the processing sample is passed, a high numerical aperture microscope objective lens that collects light emitted from processing sample as it passes through the interrogation space, an avalanche photodiode detector to detect radiation emitted from the interrogation space, and a pump to provide pressure to move the processing sample through the interrogation space, where the interrogation space is between about 0.02 pL and about 50 pL. In some embodiments, the single molecule detector used in the methods of the invention utilizes a capillary flow system, and includes a capillary flow cell, a continuous wave laser to illuminate an interrogation space in the capillary through which the processing sample is passed, a high numerical aperture microscope objective lens that collects light emitted from the processing sample as it passes through the interrogation space wherein the lens has a numerical aperture of at least about 0.8, an avalanche photodiode detector to detect radiation emitted from the interrogation space, and a pump to provide pressure to move the processing sample through the interrogation space, where the interrogation space is between about 0.004 pL and about 100 pL. In some embodiments, the single molecule detector used in the methods of the invention utilizes a capillary flow system, and includes a capillary flow cell, a continuous wave laser to illuminate an interrogation space in the capillary through which the processing sample is passed, a high numerical aperture microscope objective lens that collects light emitted from the processing sample as it passes through the interrogation space wherein the lens has a numerical aperture of at least about 0.8, an avalanche photodiode detector to detect radiation emitted from the interrogation space, and a pump to provide pressure to move a processing sample through the interrogation space, where the interrogation space is between about 0.05 pL and about 10 pL. In some embodiments, the single molecule detector used in the methods of the invention utilizes a capillary flow system, and includes a capillary flow cell, a continuous wave laser to illuminate an interrogation space in the capillary through which the processing sample is passed, a high numerical aperture microscope objective lens that collects light emitted from the processing sample as it passes through the interrogation space wherein the lens has a numerical aperture of at least about 0.8, an avalanche photodiode detector to detect radiation emitted from the interrogation space, and a pump to provide pressure to move the processing sample through the interrogation space, where the interrogation space is between about 0.05 pL and about 5 pL. In some embodiments, the single molecule detector used in the methods of the invention utilizes a capillary flow system, and includes a capillary flow cell, a continuous wave laser to illuminate an interrogation space in the capillary through which the processing sample is passed, a high numerical aperture microscope objective lens that collects light emitted from the processing sample as it passes through the interrogation space wherein the lens has a numerical aperture of at least about 0.8, an avalanche photodiode detector to detect radiation emitted from the interrogation space, and a pump to provide pressure to move the processing sample through the interrogation space, where the interrogation space is between about 0.5 pL and about 5 pL.

In some embodiments, the single molecule detector is capable of determining a concentration of a molecule of interest in a sample where sample may range in concentration over a range of at least about 100-fold, or 1000-fold, or 10,000-fold, or 100,000-fold, or 300,00-fold, or 1,000,000-fold, or 10,000,000-fold, or 30,000,000-fold.

In some embodiments, the methods of the invention utilize a single molecule detector capable detecting a difference of less than about 50%, 40%, 300%, 20%, 15%, or 10% in concentration of an analyte between a first sample and a second sample that are introduced into the detector, where the volume of the first sample and the second sample introduced into the analyzer is less than about 100, 90, 80, 70, 60, 50, 40, 30, 20, 15, 10, 5, 4, 3, 2, or 1 ul, and wherein the analyte is present at a concentration of less than about 100, 90, 80, 70, 60, 50, 40, 30, 20, 15, 10, 5, 4, 3, 2, or 1 femtomolar. In some embodiments, the methods of the invention utilize a single molecule detector capable detecting a difference of less than about 50% in concentration of an analyte between a first sample and a second sample that are introduced into the detector, where the volume of the first sample and the second sample introduced into the analyzer is less than about 100 ul, and wherein the analyte is present at a concentration of less than about 100 femtomolar. In some embodiments, the methods of the invention utilize a single molecule detector capable detecting a difference of less than about 40% in concentration of an analyte between a first sample and a second sample that are introduced into the detector, where the volume of the first sample and the second sample introduced into the analyzer is less than about 50 ul, and wherein the analyte is present at a concentration of less than about 50 femtomolar. In some embodiments, the methods of the invention utilize a single molecule detector capable detecting a difference of less than about 20% in concentration of an analyte between a first sample and a second sample that are introduced into the detector, where the volume of the first sample and the second sample introduced into the analyzer is less than about 20 ul, and wherein the analyte is present at a concentration of less than about 20 femtomolar. In some embodiments, the methods of the invention utilize a single molecule detector capable detecting a difference of less than about 20% in concentration of an analyte between a first sample and a second sample that are introduced into the detector, where the volume of the first sample and the second sample introduced into the analyzer is less than about 10 ul, and wherein the analyte is present at a concentration of less than about 10 femtomolar. In some embodiments, the methods of the invention utilize a single molecule detector capable detecting a difference of less than about 20% in concentration of an analyte between a first sample and a second sample that are introduced into the detector, where the volume of the first sample and the second sample introduced into the analyzer is less than about 5 ul, and wherein the analyte is present at a concentration of less than about 5 femtomolar.

The single molecule detector and systems are described in more detail below. Further embodiments of suitable single molecule detectors and systems useful in the methods of the invention, such as detectors with more than one interrogation window, or detectors utilizing electrokinetic or electrophoretic flow, and the like, may be found in U.S. patent application Ser. No. 11/048,660, which is incorporated by reference herein in its entirety.

Between runs the instrument may be washed. A wash buffer that maintains the salt and surfactant concentrations of the sample may be used in some embodiments to maintain the conditioning of the capillary; i.e., to keep the capillary surface relatively constant between samples to reduce variability.

A feature that contributes to the extremely high sensitivity of the instruments and methods of the invention is the method of detecting and counting labels, which, in some embodiments, are attached to single molecules to be detected or, more typically, correspond to a single molecule to be detected. Briefly, the processing sample flowing through the capillary is effectively divided into a series of detection events, by subjecting a given interrogation space of the capillary to electromagnetic (EM) radiation from a laser that emits light at an appropriate excitation wavelength for the fluorescent moiety used in the label for a predetermined period of time, and detecting photons emitted during that time. Each predetermined period of time is a “bin.” If the total number of photons detected in a given bin exceeds a predetermined threshold level, a detection event is registered for that bin, i.e., a label has been detected. If the total number of photons is not at the predetermined threshold level, no detection event is registered. In some embodiments, the processing sample concentration is dilute enough that, for a large percentage of detection events, the detection event represents only one label passing through the window, which corresponds to a single molecule of interest in the original sample, that is, few detection events represent more than one label in a single bin. In some embodiments, further refinements are applied to allow greater concentrations of label in the processing sample to be detected accurately, i.e., concentrations at which the probability of two or more labels being detected as a single detection event is no longer insignificant.

Although other bin times may be used without departing from the scope of the present invention, in some embodiments the bin times are selected in the range of about 1 microsecond to about 5 ms. In some embodiments, the bin time is more than about 1, 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is less than about 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is about 1 to about 1000 microseconds. In some embodiments, the bin time is about 1 to about 750 microseconds. In some embodiments, the bin time is about 1 to about 500 microseconds. In some embodiments, the bin time is about 1 to about 250 microseconds. In some embodiments, the bin time is about 1 to about 100 microseconds. In some embodiments, the bin time is about 1 to about 50 microseconds. In some embodiments, the bin time is about 1 to about 40 microseconds. In some embodiments, the bin time is about 1 to about 30 microseconds. In some embodiments, the bin time is about 1 to about 500 microseconds. In some embodiments, the bin time is about 1 to about 20 microseconds. In some embodiments, the bin time is about 1 to about 10 microseconds. In some embodiments, the bin time is about 1 to about 500 microseconds. In some embodiments, the bin time is about 1 to about 5 microseconds. In some embodiments, the bin time is about 5 to about 500 microseconds. In some embodiments, the bin time is about 5 to about 250 microseconds. In some embodiments, the bin time is about 5 to about 100 microseconds. In some embodiments, the bin time is about 5 to 50 microseconds. In some embodiments, the bin time is about 5 to about 20 microseconds. In some embodiments, the bin time is about 5 to about 10 microseconds. In some embodiments, the bin time is about 10 to about 500 microseconds. In some embodiments, the bin time is about 10 to about 250 microseconds. In some embodiments, the bin time is about 10 to about 100 microseconds. In some embodiments, the bin time is about 10 to about 50 microseconds. In some embodiments, the bin time is about 10 to about 30 microseconds. In some embodiments, the bin time is about 10 to about 20 microseconds. In some embodiments, the bin time is about 5 microseconds. In some embodiments, the bin time is about 5 microseconds. In some embodiments, the bin time is about 6 microseconds. In some embodiments, the bin time is about 7 microseconds. In some embodiments, the bin time is about 8 microseconds. In some embodiments, the bin time is about 9 microseconds. In some embodiments, the bin time is about 10 microseconds. In some embodiments, the bin time is about 11 microseconds. In some embodiments, the bin time is about 12 microseconds. In some embodiments, the bin time is about 13 microseconds. In some embodiments, the bin time is about 14 microseconds. In some embodiments, the bin time is about 15 microseconds. In some embodiments, the bin time is about 16 microseconds. In some embodiments, the bin time is about 17 microseconds. In some embodiments, the bin time is about 18 microseconds. In some embodiments, the bin time is about 19 microseconds. In some embodiments, the bin time is about 20 microseconds. In some embodiments, the bin time is about 25 microseconds. In some embodiments, the bin time is about 30 microseconds. In some embodiments, the bin time is about 40 microseconds. In some embodiments, the bin time is about 50 microseconds. In some embodiments, the bin time is about 100 microseconds. In some embodiments, the bin time is about 250 microseconds. In some embodiments, the bin time is about 500 microseconds. In some embodiments, the bin time is about 750 microseconds. In some embodiments, the bin time is about 1000 microseconds.

In some embodiments, the background noise level is determined from the mean noise level, or the root-mean-square noise. In some embodiments, a typical noise value or standard noise value is used. In some embodiments, a statistical value is chosen. In most cases, the noise is expected to follow a Poisson distribution. Thus, in some embodiments, determining the concentration of a particle-label complex in a sample comprises determining the background noise level.

Thus, as a label flows through the capillary flow cell, it is irradiated by the laser beam to generate a burst of photons. The photons emitted by the label are discriminated from background light or background noise emission by considering only the bursts of photons that have energy above a predetermined threshold energy level which accounts for the amount of background noise that is present in the sample. Background noise typically comprises low frequency emission produced, for example, by the intrinsic fluorescence of non-labeled particles that are present in the sample, the buffer or diluent used in preparing the sample for analysis, Raman scattering and electronic noise. In some embodiments, the value assigned to the background noise is calculated as the average background signal noise detected in a plurality of bins, which are measurements of photon signals that are detected in an interrogation space during a predetermined length of time. Thus in some embodiments, background noise is calculated for each sample as a number specific to that sample.

Given the value for the background noise, the threshold energy level can be assigned. As discussed above, the threshold value is determined to discriminate true signals (due to fluorescence of a label) from the background noise. Care must be taken in choosing a threshold value such that the number of false positive signals from random noise is minimized while the number of true signals which are rejected is also minimized. Methods for choosing a threshold value include, but are not limited to, determining a fixed value above the noise level and calculating a threshold value based on the distribution of the noise signal. In one embodiment, the threshold is set at a fixed number of standard deviations above the background level. Assuming a Poisson distribution of the noise, using this method one can estimate the number of false positive signals over the time course of the experiment. In some embodiments, the threshold level is calculated as a value of 4 sigma above the background noise. For example, given an average background noise level of 200 photons, the analyzer system establishes a threshold level of 4√200 above the average background/noise level of 200 photons to be 256 photons. Thus, in some embodiments, determining the concentration of a label in a sample includes establishing the threshold level above which photon signals represent the presence of a label. Conversely, photon signals that have an energy level that is not greater than that of the threshold level indicate the absence of a label.

Many bin measurements can be taken to determine the concentration of a sample, and the absence or presence of a label is ascertained for each bin measurement. Typically, 60,000 measurements or more can be made in one minute (e.g., in embodiments in which the bin size is 1 ms; for smaller bin sizes the number of measurements is correspondingly larger, e.g., 6,000,000 measurements per minute for a bin size of 10 microseconds). Thus, no single measurement is crucial and the method provides for a high margin of error. The bins that are determined not to contain a label (“no” bins) are discounted and only the measurements made in the bins that are determined to contain label (“yes” bins) are accounted in determining the concentration of the label in the processing sample. Discounting measurements made in the “no” bins or bins that are devoid of label increases the signal to noise ratio and the accuracy of the measurements. Thus, in some embodiments, determining the concentration of a label in a sample comprises detecting the bin measurements that reflect the presence of a label.

The signal to noise ratio or the sensitivity of the analyzer system can be increased by minimizing the time that background noise is detected during a bin measurement in which a particle-label complex is detected. For example, in a bin measurement lasting 1 millisecond during which one particle-label complex is detected when passing across an interrogation space within 250 microseconds, 750 microseconds of the 1 millisecond are spent detecting background noise emission. The signal to noise ratio can be improved by decreasing the bin time. In some embodiments, the bin time is about 1 millisecond. In other embodiments, the bin time is about 750 microseconds, 500 microseconds, 250 microseconds, 100 microseconds, 50 microseconds, 25 microseconds or 10 microseconds. Other bin times are as described herein.

Other factors that affect measurements are the brightness or dimness of the fluorescent moiety, the flow rate, and the power of the laser. Various combinations of the relevant factors that allow for detection of label will be apparent to those of skill in the art. In some embodiments, the bin time is adjusted without changing the flow rate. It will be appreciated by those of skill in the art that as bin time decreases, laser power output directed at the interrogation space must increase to maintain a constant total energy applied to the interrogation space during the bin time. For example, if bin time is decreased from 1000 microseconds to 250 microseconds, as a first approximation, laser power output must be increased approximately four-fold. These settings allow for the detection of the same number of photons in a 250 μs bin as the number of photons counted during the 1000 μs bin given the previous settings, and allow for faster analysis of sample with lower backgrounds and thus greater sensitivity. In addition, flow rates may be adjusted in order to speed processing of sample. These numbers are merely exemplary, and the skilled practitioner can adjust the parameters as necessary to achieve the desired result.

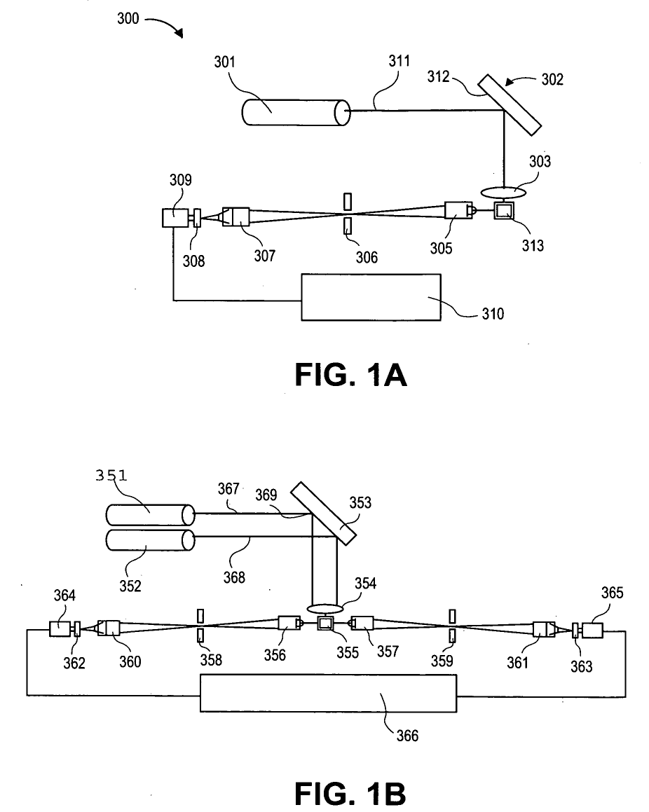

In some embodiments, the interrogation space encompasses the entire cross-section of the sample stream. When the interrogation space encompasses the entire cross-section of the sample stream, only the number of labels counted and the volume passing through a cross-section of the sample stream in a set length of time are needed to calculate the concentration of the label in the processing sample. In some embodiments, the interrogation space can be defined to be smaller than the cross-sectional area of sample stream by, for example, the interrogation space is defined by the size of the spot illuminated by the laser beam. In some embodiments, the interrogation space can be defined by adjusting the apertures 306 (FIG. 1A) or 358 and 359 (FIG. 1B) of the analyzer and reducing the illuminated volume that is imaged by the objective lens to the detector. In the embodiments when the interrogation space is defined to be smaller than the cross-sectional area of sample stream, the concentration of the label can be determined by interpolation of the signal emitted by the complex from a standard curve that is generated using one or more samples of known standard concentrations. In yet other embodiments, the concentration of the label can be determined by comparing the measured particles to an internal label standard. In embodiments where a diluted sample is analyzed, the dilution factor is accounted in calculating the concentration of the molecule of interest in the starting sample.

As discussed above, when the interrogation space encompasses the entire cross-section of the sample stream, only the number of labels counted passing through a cross-section of the sample stream in a set length of time (bin) and the volume of sample that was interrogated in the bin are needed to calculate the concentration the sample. The total number of labels contained in the “yes” bins is determined and related to the sample volume represented by the total number of bins used in the analysis to determine the concentration of labels in the processing sample. Thus, in one embodiment, determining the concentration of a label in a processing sample comprises determining the total number of labels detected “yes” bins and relating the total number of detected labels to the total sample volume that was analyzed. The total sample volume that is analyzed is the sample volume that is passed through the capillary flow cell and across the interrogation space in a specified time interval. Alternatively, the concentration of the label complex in a sample is determined by interpolation of the signal emitted by the label in a number of bins from a standard curve that is generated by determining the signal emitted by labels in the same number of bins by standard samples containing known concentrations of the label.

In some embodiments, the number of individual labels that are detected in a bin is related to the relative concentration of the particle in the processing sample. At relatively low concentrations, for example at concentrations below about 10-16 M the number of labels is proportional to the photon signal that is detected in a bin. Thus, at low concentrations of label the photon signal is provided as a digital signal. At relatively higher concentrations, for example at concentrations greater than about 10-16 M, the proportionality of photon signal to a label is lost as the likelihood of two or more labels crossing the interrogation space at about the same time and being counted as one becomes significant. Thus, in some embodiments, individual particles in a sample of a concentration greater than about 10-16 M are resolved by decreasing the length of time of the bin measurement.

Alternatively, in other embodiments, the total photon signal that is emitted by a plurality of particles that are present in any one bin is detected. These embodiments allow for single molecule detectors of the invention wherein the dynamic range is at least 3, 3.5, 4, 4.5, 5.5, 6, 6.5, 7, 7.5, 8, or more than 8 logs.

“Dynamic range,” as that term is used herein, refers to the range of sample concentrations that may be quantitated by the instrument without need for dilution or other treatment to alter the concentration of successive samples of differing concentrations, where concentrations are determined with an accuracy appropriate for the intended use. For example, if a microtiter plate contains a sample of 1 femtomolar concentration for an analyte of interest in one well, a sample of 10,000 femtomolar concentration for an analyte of interest in another well, and a sample of 100 femtomolar concentration for the analyte in a third well, an instrument with a dynamic range of at least 4 logs and a lower limit of quantitation of 1 femtomolar is able to accurately quantitate the concentration of all the samples without the need for further treatment to adjust concentration, e.g., dilution. Accuracy may be determined by standard methods, e.g., using a series of standards of concentrations that span the dynamic range and constructing a standard curve. Standard measures of fit of the resulting standard curve may be used as a measure of accuracy, e.g., an r² greater than about 0.7, 0.75, 0.8, 0.85, 0.9, 0.91, 0.92, 0.93, 0.94, 0.95, 0.96, 0.97, 0.98, or 0.99.

Increased dynamic range is achieved by altering the manner in which data from the detector is analyzed, and/or by the use of an attenuator between the detector and the interrogation space. At the low end of the range, where the processing sample is sufficiently dilute that each detection event, i.e., each burst of photons above a threshold level in a bin (the “event photons”), likely represents only one label, the data is analyzed to count detection events as single molecules; i.e., each bin is analyzed as a simple “yes” or “no” for the presence of label, as described above. For a more concentrated processing sample, where the likelihood of two or more labels occupying a single bin becomes significant, the number of event photons in a significant number of bins is found to be substantially greater than the number expected for a single label, e.g., the number of event photons in a significant number of bins corresponds to two-fold, three-fold, or more, than the number of event photons expected for a single label. For these samples, the instrument changes its method of data analysis to one of integrating the total number of event photons for the bins of the processing sample. This total will be proportional to the total number of labels that were in all the bins. For an even more concentrated processing sample, where many labels are present in most bins, background noise becomes an insignificant portion of the total signal from each bin, and the instrument changes its method of data analysis to one of counting total photons per bin (including background). An even further increase in dynamic range can be achieved by the use of an attenuator between the flow cell and the detector, when concentrations are such that the intensity of light reaching the detector would otherwise exceed the capacity of the detector for accurately counting photons, i.e., saturate the detector.

The instrument may include a data analysis system that receives input from the detector and determines the appropriate analysis method for the sample being run, and outputs values based on such analysis. The data analysis system may further output instructions to use or not use an attenuator, if an attenuator is included in the instrument. By utilizing such methods, the dynamic range of the instrument can be dramatically increased. Thus, in some embodiments, the instrument is capable of measuring concentrations of samples over a dynamic range of more than about 1000 (3 log), 10,000 (4 log), 100,000 (5 log), 350,000 (5.5 log), 1,000,000 (6 log), 3,500,000 (6.5 log), 10,000,000 (7 log), 35,000,000 (7.5 log), or 100,000,000 (8 log). In some embodiments, the instrument is capable of measuring concentrations of samples over a dynamic range of more than about 100,000 (5 log). In some embodiments, the instrument is capable of measuring concentrations of samples over a dynamic range of more than about 1,000,000 (6 log). In some embodiments, the instrument is capable of measuring concentrations of samples over a dynamic range of more than about 10,000,000 (7 log). In some embodiments, the instrument is capable of measuring the concentrations of samples over a dynamic range of from about 1-10 femtomolar to at least about 1000; 10,000; 100,000; 350,000; 1,000,000; 3,500,000; 10,000,000, or 35,000,000 femtomolar. In some embodiments, the instrument is capable of measuring the concentrations of samples over a dynamic range of from about 1-10 femtomolar to at least about 10,000 femtomolar. In some embodiments, the instrument is capable of measuring the concentrations of samples over a dynamic range of from about 1-10 femtomolar to at least about 100,000 femtomolar. In some embodiments, the instrument is capable of measuring the concentrations of samples over a dynamic range of from about 1-10 femtomolar to at least about 1,000,000 femtomolar. In some embodiments, the instrument is capable of measuring the concentrations of samples over a dynamic range of from about 1-10 femtomolar to at least about 10,000,000.

In some embodiments, an analyzer or analyzer system of the invention is capable of detecting an analyte, e.g., a biomarker, at a limit of detection of less than about 1 nanomolar, 1 picomolar, 1 femtomolar, 1 attomolar, 1 zeptomolar. In some embodiments, the analyzer or analyzer system is capable of detecting a change in concentration of the analyte, or of multiple analytes, e.g., a biomarker or biomarkers, from one sample to another sample of less than about 0.1, 1, 2, 5, 10, 20, 30, 40, 50, 60, or 80% when the biomarker is present at a concentration of less than about 1 nanomolar, 1 picomolar, 1 femtomolar, 1 attomolar, 1 zeptomolar in the samples, and when the size of each of the sample is less than about 100, 50, 40, 30, 20, 10, 5, 2, 1, 0.1, 0.01, 0.001, or 0.0001 ul. In some embodiments, the analyzer or analyzer system is capable of detecting a change in concentration of the analyte from a first sample to a second sample of less than about 20%, when the analyte is present at a concentration of less than about 1 picomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 100 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 50 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 5 μl. In some embodiments, the analyzer or analyzer system is capable of detecting a change in concentration of the analyte from a first sample to a second sample of less than about 20%, when the analyte is present at a concentration of less than about 1 femtomolar, and when the size of each of the samples is less than about 5 μl.

### V. Instruments and Systems Suitable for Highly Sensitive Analysis of Prostate Specific Antigen

The methods of the invention utilize analytical instruments of high sensitivity, e.g., single molecule detectors. Such single molecule detectors include embodiments as hereinafter described.

A. Apparatus/System

In one aspect, the methods described herein utilize an analyzer system capable of detecting a single particle in a sample. In one embodiment, the analyzer system is capable of single particle detection of a fluorescently labeled particle wherein the analyzer system detects energy emitted by an excited fluorescent label in response to exposure by an electromagnetic radiation source when the single particle is present in an interrogation space defined within a capillary flow cell fluidly connected to the sampling system of the analyzer system. In a further embodiment of the analyzer system, the single particle moves through the interrogation space of the capillary flow cell by means of a motive force. In another embodiment of the analyzer system, an automatic sampling system may be included in the analyzer system for introducing the sample into the analyzer system. In another embodiment of the analyzer system, a sample preparation system may be included in the analyzer system for preparing a sample. In a further embodiment, the analyzer system may contain a sample recovery system for recovering at least a portion of the sample after analysis is complete.

In one aspect, the analyzer system consists of an electromagnetic radiation source for exciting a single particle labeled with a fluorescent label. In one embodiment, the electromagnetic radiation source of the analyzer system is a laser. In a further embodiment, the electromagnetic radiation source is a continuous wave laser.

In a typical embodiment, the electromagnetic radiation source excites a fluorescent moiety attached to a label as the label passes through the interrogation space of the capillary flow cell. In some embodiments, the fluorescent label moiety includes one or more fluorescent dye molecules. In some embodiments, the fluorescent label moiety is a quantum dot. Any fluorescent moiety as described herein may be used in the label.

A label is exposed to electromagnetic radiation when the label passes through an interrogation space located within the capillary flow cell. The interrogation space is typically fluidly connected to a sampling system. In some embodiments the label passes through the interrogation space of the capillary flow cell due to a motive force to advance the label through the analyzer system. The interrogation space is positioned such that it receives electromagnetic radiation emitted from the radiation source. In some embodiments, the sampling system is an automated sampling system capable of sampling a plurality of samples without intervention from a human operator.

The label passes through the interrogation space and emits a detectable amount of energy when excited by the electromagnetic radiation source. In one embodiment, an electromagnetic radiation detector is operably connected to the interrogation space. The electromagnetic radiation detector is capable of detecting the energy emitted by the label, e.g., by the fluorescent moiety of the label.

In a further embodiment of the analyzer system, the system further includes a sample preparation mechanism where a sample may be partially or completely prepared for analysis by the analyzer system. In some embodiments of the analyzer system, the sample is discarded after it is analyzed by the system. In other embodiments, the analyzer system further includes a sample recovery mechanism whereby at least a portion, or alternatively all or substantially all, of the sample may be recovered after analysis. In such an embodiment, the sample can be returned to the origin of the sample. In some embodiments, the sample can be returned to microtiter wells on a sample microtiter plate. The analyzer system typically further consists of a data acquisition system for collecting and reporting the detected signal.

B. Single Particle Analyzer

As shown in FIG. 1A, described herein is one embodiment of an analyzer system 300. The analyzer system 300 includes an electromagnetic radiation source 301, a mirror 302, a lens 303, a capillary flow cell 313, a microscopic objective lens 305, an aperture 306, a detector lens 307, a detector filter 308, a single photon detector 309, and a processor 310 operatively connected to the detector.

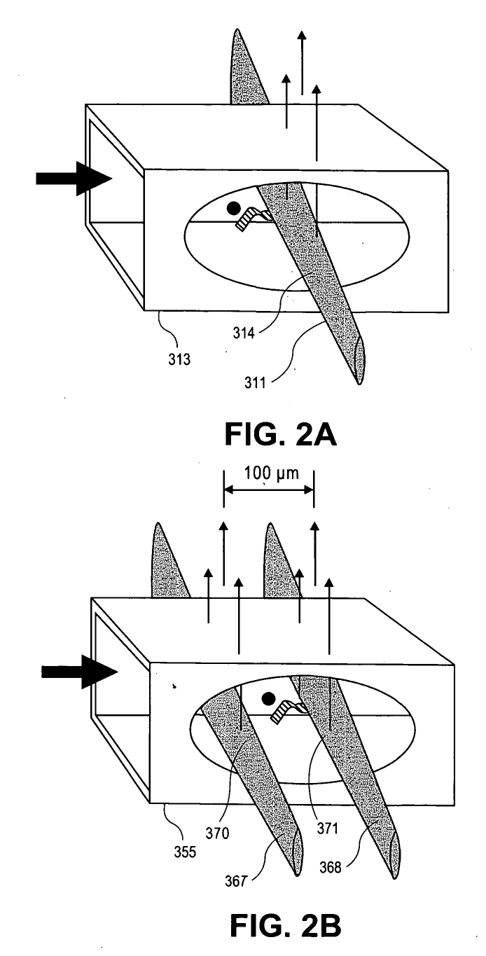

In operation the electromagnetic radiation source 301 is aligned so that its output beam 311 is reflected off of a front surface 312 of mirror the 302. The lens 303 focuses the beam 311 onto a single interrogation space (an illustrative example of an interrogation space 314 is shown in FIG. 2A) in the capillary flow cell 313. The microscope objective lens 305 collects light from sample particles and forms images of the beam onto the aperture 306. The aperture 306 affects the fraction of light emitted by the specimen in the interrogation space of the capillary flow cell 313 that can be collected. The detector lens 307 collects the light passing through the aperture 306 and focuses the light onto an active area of the detector 309 after it passes through the detector filters 308. The detector filters 308 minimize aberrant noise signals due to light scatter or ambient light while maximizing the signal emitted by the excited fluorescent moiety bound to the particle. The processor 310 processes the light signal from the particle according to the methods described herein.

In one embodiment, the microscope objective lens 305 is a high numerical aperture microscope objective. As used herein, “high numerical aperture lens” include a lens with a numerical aperture of equal to or greater than about 0.6. The numerical aperture is a measure of the number of highly diffracted image-forming light rays captured by the objective. A higher numerical aperture allows increasingly oblique rays to enter the objective lens and thereby produce a more highly resolved image. Additionally, the brightness of an image increases with a higher numerical aperture. High numerical aperture lenses are commercially available from a variety of vendors, and any one lens having a numerical aperture of equal to or greater than approximately 0.6 may be used in the analyzer system. In some embodiments, the lens has a numerical aperture of about 0.6 to about 1.3. In some embodiments, the lens has a numerical aperture of about 0.6 to about 1.0. In some embodiments, the lens has a numerical aperture of about 0.7 to about 1.2. In some embodiments, the lens has a numerical aperture of about 0.7 to about 1.0. In some embodiments, the lens has a numerical aperture of about 0.7 to about 0.9. In some embodiments, the lens has a numerical aperture of about 0.8 to about 1.3. In some embodiments, the lens has a numerical aperture of about 0.8 to about 1.2. In some embodiments, the lens has a numerical aperture of about 0.8 to about 1.0. In some embodiments, the lens has a numerical aperture of at least about 0.6. In some embodiments, the lens has a numerical aperture of at least about 0.7. In some embodiments, the lens has a numerical aperture of at least about 0.8. In some embodiments, the lens has a numerical aperture of at least about 0.9. In some embodiments, the lens has a numerical aperture of at least about 1.0. In some embodiments, the aperture of the microscope objective lens 305 is approximately 1.25. In an embodiment where a microscope objective lens 305 of 0.8 is used, a Nikon 60X/0.8 NA Achromat lens (Nikon, Inc., USA) can be used.

In some embodiments, the electromagnetic radiation source 301 is a laser that emits light in the visible spectrum. In all embodiments, the electromagnetic radiation source is set such that wavelength of the laser is set such that it is of a sufficient wavelength to excite the fluorescent label attached to the particle. In some embodiments, the laser is a continuous wave laser with a wavelength of 639 nm. In other embodiments, the laser is a continuous wave laser with a wavelength of 532 nm. In other embodiments, the laser is a continuous wave laser with a wavelength of 422 nm. In other embodiments, the laser is a continuous wave laser with a wavelength of 405 nm. Any continuous wave laser with a wavelength suitable for exciting a fluorescent moiety as used in the methods and compositions of the invention may be used without departing from the scope of the invention.

In a single particle analyzer system 300, as each particle passes through the beam 311 of the electromagnetic radiation source, the particle enters into an excited state. When the particle relaxes from its excited state, a detectable burst of light is emitted. The excitation-emission cycle is repeated many times by each particle in the length of time it takes for it to pass through the beam allowing the analyzer system 300 to detect tens to thousands of photons for each particle as it passes through an interrogation space 314. Photons emitted by fluorescent particles are registered by the detector 309 (FIG. 1A) with a time delay indicative of the time for the particle label complex to pass through the interrogation space. The photon intensity is recorded by the detector 309 and sampling time is divided into bins, which are uniform, arbitrary, time segments with freely selectable time channel widths. The number of signals contained in each bin evaluated. One or a combination of several statistical analytical methods are employed in order to determine when a particle is present. Such methods include determining the baseline noise of the analyzer system and setting a signal strength for the fluorescent label at a statistical level above baseline noise to eliminate false positive signals from the detector.

The electromagnetic radiation source 301 is focused onto a capillary flow cell 313 of the analyzer system 300 where the capillary flow cell 313 is fluidly connected to the sample system. An interrogation space 314 is shown in FIG. 2A. The beam 311 from the continuous wave electromagnetic radiation source 301 of FIG. 1A is optically focused to a specified depth within the capillary flow cell 313. The beam 311 is directed toward the sample-filled capillary flow cell 313 at an angle perpendicular to the capillary flow cell 313. The beam 311 is operated at a predetermined wavelength that is selected to excite a particular fluorescent label used to label the particle of interest. The size or volume of the interrogation space 314 is determined by the diameter of the beam 311 together with the depth at which the beam 311 is focused. Alternatively, the interrogation space can be determined by running a calibration sample of known concentration through the analyzer system.

When single molecules are detected in the sample concentration, the beam size and the depth of focus required for single molecule detection are set and thereby define the size of the interrogation space 314. The interrogation space 314 is set such that, with an appropriate sample concentration, only one particle is present in the interrogation space 314 during each time interval over which time observations are made. It will be appreciated that the detection interrogation volume as defined by the beam is not perfectly spherically shaped, and typically is a “bow-tie” shape. However, for the purposes of definition, “volumes” of interrogation spaces are defined herein as the volume encompassed by a sphere of a diameter equal to the focused spot diameter of the beam. The focused spot of the beam 311 may have various diameters without departing from the scope of the present invention. In some embodiments, the diameter of the focused spot of the beam is about 1 to about 5, 10, 15, or 20 microns, or about 5 to about 10, 15, or 20 microns, or about 10 to about 20 microns, or about 10 to about 15 microns. In some embodiments, the diameter of the focused spot of the beam is about 1, 2, 3, 4, 5, 6, 7, 8, 9, 10, 11, 12, 13, 14, 15, 16, 17, 18, 19, or 20 microns. In some embodiments, the diameter of the focused spot of the beam is about 5 microns. In some embodiments, the diameter of the focused spot of the beam is about 10 microns. In some embodiments, the diameter of the focused spot of the beam is about 12 microns. In some embodiments, the diameter of the focused spot of the beam is about 13 microns. In some embodiments, the diameter of the focused spot of the beam is about 14 microns. In some embodiments, the diameter of the focused spot of the beam is about 15 microns. In some embodiments, the diameter of the focused spot of the beam is about 16 microns. In some embodiments, the diameter of the focused spot of the beam is about 17 microns. In some embodiments, the diameter of the focused spot of the beam is about 18 microns. In some embodiments, the diameter of the focused spot of the beam is about 19 microns. In some embodiments, the diameter of the focused spot of the beam is about 20 microns.

In an alternate embodiment of the single particle analyzer system, more than one electromagnetic radiation source can be used to excite particles labeled with fluorescent labels of different wavelengths. In an alternate embodiment, more than one interrogation space in the capillary flow cell can be used. In another alternate embodiment, multiple detectors can be employed to detect different emission wavelengths from the fluorescent labels. An illustration incorporating each of these alternative embodiments of an analyzer system is shown in FIG. 1B. These embodiments are incorporated by reference from previous U.S. patent application Ser. No. 11/048,660.

In some embodiments of the analyzer system 300, a motive force is required to move a particle through the capillary flow cell 313 of the analyzer system 300. In one embodiment, the motive force can be a form of pressure. The pressure used to move a particle through the capillary flow cell can be generated by a pump. In some embodiments, a Scivex, Inc. HPLC pump can be used. In some embodiments where a pump is used as a motive force, the sample can pass through the capillary flow cell at a rate of 1 about μL/min to about 20 μL/min, or about 5 μL/min to about 20 μL/min. In some embodiments, the sample can pass through the capillary flow cell at a rate of about 5 μL/min. In some embodiments, the sample can pass through the capillary flow cell at a rate of about 10 μL/min. In some embodiments, the sample can pass through the capillary flow cell at a rate of about 15 μL/min. In some embodiments, the sample can pass through the capillary flow cell at a rate of about 20 μL/min. In some embodiments, an electrokinetic force can be used to move the particle through the analyzer system. Such a method has been previously disclosed and is incorporated by reference from previous U.S. patent application Ser. No. 11/048,660.

In one aspect of the analyzer system 300, the detector 309 of the analyzer system detects the photons emitted by the fluorescent label. In one embodiment, the photon detector is a photodiode. In a further embodiment, the detector is an avalanche photodiode detector. In some embodiments, the photodiodes can be silicon photodiodes with a wavelength detection of 190 nm and 1100 nm. When germanium photodiodes are used, the wavelength of light detected is between 400 nm to 1700 nm. In other embodiments, when an indium gallium arsenide photodiode is used, the wavelength of light detected by the photodiode is between 800 nm and 2600 nm. When lead sulfide photodiodes are used as detectors, the wavelength of light detected is between 1000 nm and 3500 nm.

In some embodiments, the optics of the electromagnetic radiation source 301 and the optics of the detector 309 are arranged in a conventional optical arrangement. In such an arrangement, the electromagnetic radiation source and the detector are aligned on different focal planes. The arrangement of the laser and the detector optics of the analyzer system as shown in FIGS. 1A and 1B is that of a conventional optical arrangement.

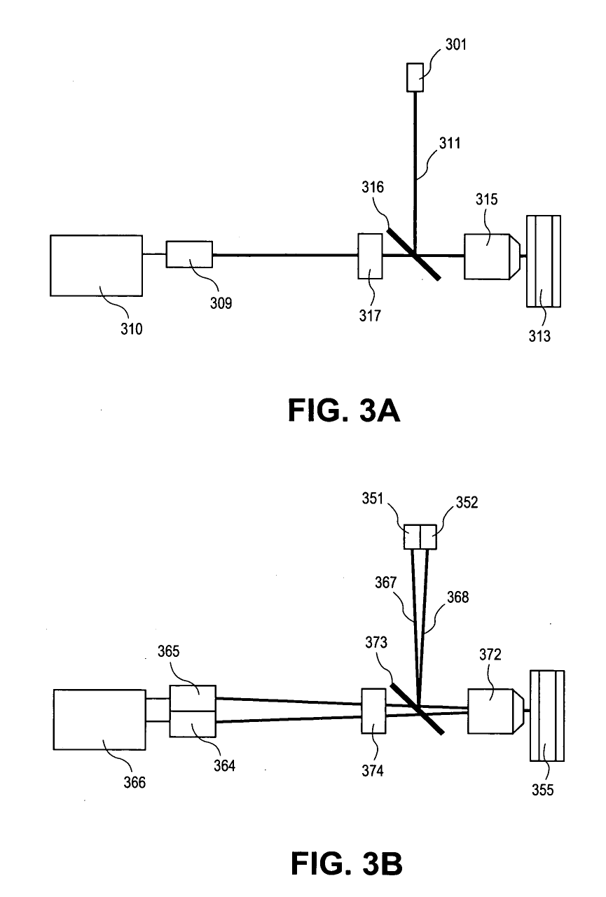

In some embodiments, the optics of the electromagnetic radiation source and the optics of the detector are arranged in a confocal optical arrangement. In such an arrangement, the electromagnetic radiation source 301 and the detector 309 are aligned on the same focal plane. The confocal arrangement renders the analyzer more robust because the electromagnetic radiation source 301 and the detector optics 309 do not need to be realigned if the analyzer system is moved. This arrangement also makes the use of the analyzer more simplified because it eliminates the need to realign the components of the analyzer system. The confocal arrangement for the analyzer 300 (FIG. 1A) and the analyzer 355 (FIG. 1B) are shown in FIGS. 3A and 3B respectively. FIG. 3A shows that the beam 311 from an electromagnetic radiation source 301 is focused by the microscope objective 315 to form one interrogation space 314 (FIG. 2A) within the capillary flow cell 313. A dichroic mirror 316, which reflects laser light but passes fluorescent light, is used to separate the fluorescent light from the laser light. Filter 317 that is positioned in front of the detector eliminates any non-fluorescent light at the detector. In some embodiments, an analyzer system configured in a confocal arrangement can comprise two or more interrogations spaces. Such a method has been previously disclosed and is incorporated by reference from previous U.S. patent application Ser. No. 11/048,660.

The laser can be a tunable dye laser, such as a helium-neon laser. The laser can be set to emit a wavelength of 632.8 nm. Alternatively, the wavelength of the laser can be set to emit a wavelength of 543.5 nm or 1523 nm. Alternatively, the electromagnetic laser can be an argon ion laser. In such an embodiment, the argon ion laser can be operated as a continuous gas laser at about 25 different wavelengths in the visible spectrum, the wavelength set between 408.9 and 686.1 nm but at its optimum performance set between 488 and 514.5 nm.

1. Electromagnetic Radiation Source

In some embodiments of the analyzer system a chemiluminescent label may be used. In such an embodiment, it may not be necessary to utilize an electromagnetic (EM) source for detection of the particle. In another embodiment, the extrinsic label or intrinsic characteristic of the particle is a light-interacting label or characteristic, such as a fluorescent label or a light-scattering label. In such an embodiment, a source of EM radiation is used to illuminate the label and/or the particle. Any suitable EM radiation source for excitation of fluorescent labels can be used.

In some embodiments, the analyzer system consists of an electromagnetic radiation source 301. Any number of radiation sources may be used in any one analyzer system 300 without departing from the scope of the invention. Multiple sources of electromagnetic radiation have been previously disclosed and are incorporated by reference from previous U.S. patent application Ser. No. 11/048,660. In some embodiments, all the continuous wave electromagnetic (EM) radiation sources emit electromagnetic radiation at the same wavelengths. In other embodiments, different sources emit different wavelengths of EM radiation.

In one embodiment, the EM source(s) 301, 351, 352 are continuous wave lasers producing wavelengths of between 200 nm and 1000 nm. Such EM sources have the advantage of being small, durable and relatively inexpensive. In addition, they generally have the capacity to generate larger fluorescent signals than other light sources. Specific examples of suitable continuous wave EM sources include, but are not limited to: lasers of the argon, krypton, helium-neon, helium-cadmium types, as well as tunable diode lasers (red to infrared regions), each with the possibility of frequency doubling. The lasers provide continuous illumination with no accessory electronic or mechanical devices, such as shutters, to interrupt their illumination. In an embodiment where a continuous wave laser is used, an electromagnetic radiation source of 3 mW may be of sufficient energy to excite a fluorescent label. A beam from a continuous wave laser of such energy output may be between about 2 to about 5 μm in diameter. The time of exposure of the particle to the laser beam in order to be exposed to 3 mW may be a time period of about 1 msec. In alternate embodiments, the time of exposure to the laser beam may be equal to or less than about 500 μsec. In an alternate embodiment, the time of exposure may be equal to or less than about 100 μsec. In an alternate embodiment, the time of exposure may be equal to or less than about 50 μsec. In an alternate embodiment, the time of exposure may be equal to or less than about 10 μsec.

LEDs are another low-cost, high reliability suitable illumination source. Recent advances in ultra-bright LEDs and dyes, with high absorption cross-section and quantum yield, support the applicability of LEDs to single particle detection. Such lasers could be used alone or in combination with other light sources such as mercury arc lamps, elemental arc lamps, halogen lamps, arc discharges, plasma discharges, light-emitting diodes, or combination of these.

In some embodiments, the EM source could be in the form of a pulse wave laser. In such an embodiment, the pulse size of the laser is an important factor. In such an embodiment, the size, focus spot, and the total energy emitted by the laser is important and must be of sufficient energy as to be able to excite the fluorescent label. When a pulse laser is used, a pulse of longer duration may be required. In some embodiments a laser pulse of about 2 nanoseconds may be used. In some embodiments a laser pulse of about 5 nanoseconds may be used. In some embodiments a pulse of between about 2 to about 5 nanoseconds may be used.

The optimal laser intensity depends on the photo bleaching characteristics of the single dyes and the length of time required to traverse the interrogation space (including the speed of the particle, the distance between interrogation spaces if more than one is used and the size of the interrogation space(s)). To obtain a maximal signal, it is desirable to illuminate the sample at the highest intensity which will not result in photo bleaching a high percentage of the dyes. The preferred intensity is one such that no more than about 5% of the dyes are bleached by the time the particle has traversed the interrogation space.

The power of the laser is set depending on the type of dye molecules that need to be stimulated and the length of time the dye molecules are stimulated, and/or the speed with which the dye molecules pass through the capillary flow cell. Laser power is defined as the rate at which energy is delivered by the beam and is measured in units of Joules/second, or Watts. It will be appreciated that the greater the power output of the laser, the shorter the time that the laser illuminates the particle may be, while providing a constant amount of energy to the interrogation space while the particle is passing through the space. Thus, in some embodiments, the combination of laser power and time of illumination is such that the total energy received by the interrogation space during the time of illumination is more than about 0.1, 0.5, 1, 2, 3, 4, 5, 6, 7, 8, 9, 10, 12, 15, 20, 25, 30, 35, 40, 45, 50, 60, 70, 80, 90, or 100 microJoule. In some embodiments, the combination of laser power and time of illumination is such that the total energy received by the interrogation space during the time of illumination is less than about 0.5, 1, 2, 3, 4, 5, 6, 7, 8, 9, 10, 12, 15, 20, 25, 30, 35, 40, 45, 50, 60, 70, 80, 90, 100, or 110 microJoule. In some embodiments, the combination of laser power and time of illumination is such that the total energy received by the interrogation space during the time of illumination is between about 0.1 and about 100 microJoule. In some embodiments, the combination of laser power and time of illumination is such that the total energy received by the interrogation space during the time of illumination is between about 1 and about 100 microJoule. In some embodiments, the combination of laser power and time of illumination is such that the total energy received by the interrogation space during the time of illumination is between about 1 and about 50 microJoule. In some embodiments, the combination of laser power and time of illumination is such that the total energy received by the interrogation space during the time of illumination is between about 2 and about 50 microJoule. In some embodiments, the combination of laser power and time of illumination is such that the total energy received by the interrogation space during the time of illumination is between about 3 and about 60 microJoule. In some embodiments, the combination of laser power and time of illumination is such that the total energy received by the interrogation space during the time of illumination is between about 3 and about 50 microJoule. In some embodiments, the combination of laser power and time of illumination is such that the total energy received by the interrogation space during the time of illumination is between about 3 and about 40 microJoule. In some embodiments, the combination of laser power and time of illumination is such that the total energy received by the interrogation space during the time of illumination is between about 3 and about 30 microJoule. In some embodiments, the combination of laser power and time of illumination is such that the total energy received by the interrogation space during the time of illumination is about 1 microJoule. In some embodiments, the combination of laser power and time of illumination is such that the total energy received by the interrogation space during the time of illumination is about 3 microJoule. In some embodiments, the combination of laser power and time of illumination is such that the total energy received by the interrogation space during the time of illumination is about 5 microJoule. In some embodiments, the combination of laser power and time of illumination is such that the total energy received by the interrogation space during the time of illumination is about 10 microJoule. In some embodiments, the combination of laser power and time of illumination is such that the total energy received by the interrogation space during the time of illumination is about 15 microJoule. In some embodiments, the combination of laser power and time of illumination is such that the total energy received by the interrogation space during the time of illumination is about 20 microJoule. In some embodiments, the combination of laser power and time of illumination is such that the total energy received by the interrogation space during the time of illumination is about 30 microJoule. In some embodiments, the combination of laser power and time of illumination is such that the total energy received by the interrogation space during the time of illumination is about 40 microJoule. In some embodiments, the combination of laser power and time of illumination is such that the total energy received by the interrogation space during the time of illumination is about 50 microJoule. In some embodiments, the combination of laser power and time of illumination is such that the total energy received by the interrogation space during the time of illumination is about 60 microJoule. In some embodiments, the combination of laser power and time of illumination is such that the total energy received by the interrogation space during the time of illumination is about 70 microJoule. In some embodiments, the combination of laser power and time of illumination is such that the total energy received by the interrogation space during the time of illumination is about 80 microJoule. In some embodiments, the combination of laser power and time of illumination is such that the total energy received by the interrogation space during the time of illumination is about 90 microJoule. In some embodiments, the combination of laser power and time of illumination is such that the total energy received by the interrogation space during the time of illumination is about 100 microJoule.

In some embodiments, the laser power output is set to at least about 1 mW, 2 mW, 3 mW, 4 mW, 5 mW, 6, mW, 7 mW, 8 mW, 9 mW, 10 mW, 13 mW, 15 mW, 20 mW, 25 mW, 30 mW, 40 mW, 50 mW, 60 mW, 70 mW, 80 mW, 90 mW, 100 mW, or more than 100 mW. In some embodiments, the laser power output is set to at least about 1 mW. In some embodiments, the laser power output is set to at least about 3 mW. In some embodiments, the laser power output is set to at least about 5 mW. In some embodiments, the laser power output is set to at least about 10 mW. In some embodiments, the laser power output is set to at least about 20 mW. In some embodiments, the laser power output is set to at least about 30 mW. In some embodiments, the laser power output is set to at least about 40 mW. In some embodiments, the laser power output is set to at least about 50 mW. In some embodiments, the laser power output is set to at least about 60 mW. In some embodiments, the laser power output is set to at least about 90 mW.

The time that the laser illuminates the interrogation space can be set to no less than about 1, 2, 3, 4, 5, 10, 15, 20, 30, 40, 50, 60, 70, 80, 90, 100, 150, 200, 150, 300, 350, 400, 450, 500, 600, 700, 800, 900, or 1000 microseconds. The time that the laser illuminates the interrogation space can be set to no more than about 2, 3, 4, 5, 10, 15, 20, 30, 40, 50, 60, 70, 80, 90, 100, 150, 200, 150, 300, 350, 400, 450, 500, 600, 700, 800, 900, 1000, 1500, or 2000 microseconds. The time that the laser illuminates the interrogation space can be set between about 1 and about 1000 microseconds. The time that the laser illuminates the interrogation space can be set between about 5 and about 500 microseconds. The time that the laser illuminates the interrogation space can be set between about 5 and about 100 microseconds. The time that the laser illuminates the interrogation space can be set between about 10 and about 100 microseconds. The time that the laser illuminates the interrogation space can be set between about 10 and about 50 microseconds. The time that the laser illuminates the interrogation space can be set between about 10 and about 20 microseconds. The time that the laser illuminates the interrogation space can be set between about 5 and about 50 microseconds. The time that the laser illuminates the interrogation space can be set between about 1 and about 100 microseconds. In some embodiments, the time that the laser illuminates the interrogation space is about 1 microsecond. In some embodiments, the time that the laser illuminates the interrogation space is about 5 microseconds. In some embodiments, the time that the laser illuminates the interrogation space is about 10 microseconds. In some embodiments, the time that the laser illuminates the interrogation space is about 25 microseconds. In some embodiments, the time that the laser illuminates the interrogation space is about 50 microseconds. In some embodiments, the time that the laser illuminates the interrogation space is about 100 microseconds. In some embodiments, the time that the laser illuminates the interrogation space is about 250 microseconds. In some embodiments, the time that the laser illuminates the interrogation space is about 500 microseconds. In some embodiments, the time that the laser illuminates the interrogation space is about 1000 microseconds.

For example, the time that the laser illuminates the interrogation space can be set to about 1 millisecond, 250 microseconds, 100 microseconds, 50 microseconds, 25 microseconds or 10 microseconds with a laser that provides a power output of about 3 mW, 4 mW, 5 mW, or more than 5 mW. In some embodiments, a label is illuminated with a laser that provides a power output of about 3 mW and illuminates the label for about 1000 microseconds. In other embodiments, a label is illuminated for less than about 1000 milliseconds with a laser providing a power output of not more than about 20 mW. In other embodiments, the label is illuminated with a laser power output of about 20 mW for less than or equal to about 250 microseconds. In some embodiments, the label is illuminated with a laser power output of about 5 mW for less than or equal to about 1000 microseconds.

2. Capillary Flow Cell

The capillary flow cell is fluidly connected to the sample system. In one embodiment, the interrogation space 314 of an analyzer system, is determined by the cross sectional area of the corresponding beam 311 and by a segment of the beam within the field of view of the detector 309. In one embodiment of the analyzer system, the interrogation space 314 has a volume, as defined herein, of between about between about 0.01 and about 500 pL, or between about 0.01 pL and about 100 pL, or between about 0.01 pL and about 10 pL, or between about 0.01 pL and about 1 pL, or between about 0.01 pL and about 0.5 pL, or between about 0.02 pL and about 300 pL, or between about 0.02 pL and about 50 pL or between about 0.02 pL and about 5 pL or between about 0.02 pL and about 0.5 pL or between about 0.02 pL and about 2 pL, or between about 0.05 pL and about 50 pL, or between about 0.05 pL and about 5 pL, or between about 0.05 pL and about 0.5 pL, or between about 0.05 pL and about 0.2 pL, or between about 0.1 pL and about 25 pL. In some embodiments, the interrogation space has a volume between about 0.01 pL and about 10 pL. In some embodiments, the interrogation space 314 has a volume between about 0.01 pL and about 1 pL. In some embodiments, the interrogation space 314 has a volume between about 0.02 pL and about 5 pL. In some embodiments, the interrogation space 314 has a volume between about 0.02 pL and about 0.5 pL. In some embodiments, the interrogation space 314 has a volume between about 0.05 pL and about 0.2 pL. In some embodiments, the interrogation space 314 has a volume of about 0.1 pL. Other useful interrogation space volumes are as described herein. It should be understood by one skilled in the art that the interrogation space 314 can be selected for maximum performance of the analyzer. Although very small interrogation spaces have been shown to minimize the background noise, large interrogation spaces have the advantage that low concentration samples can be analyzed in a reasonable amount of time. In embodiments in which two interrogation spaces 370 and 371 are used, volumes such as those described herein for a single interrogation space 314 may be used.

In one embodiment of the present invention, the interrogation spaces are large enough to allow for detection of particles at concentrations ranging from about 1000 femtomolar (fM) to about 1 zeptomolar (zM). In one embodiment of the present invention, the interrogation spaces are large enough to allow for detection of particles at concentrations ranging from about 1000 fM to about 1 attomolar (aM). In one embodiment of the present invention, the interrogation spaces are large enough to allow for detection of particles at concentrations ranging from about 10 fM to about 1 attomolar (aM). In many cases, the large interrogation spaces allow for the detection of particles at concentrations of less than about 1 fM without additional pre-concentration devices or techniques. One skilled in the art will recognize that the most appropriate interrogation space size depends on the brightness of the particles to be detected, the level of background signal, and the concentration of the sample to be analyzed.

The size of the interrogation space 314 can be limited by adjusting the optics of the analyzer. In one embodiment, the diameter of the beam 311 can be adjusted to vary the volume of the interrogation space 314. In another embodiment, the field of view of the detector 309 can be varied. Thus, the source 301 and the detector 309 can be adjusted so that single particles will be illuminated and detected within the interrogation space 314. In another embodiment, the width of aperture 306 (FIG. 1A) that determine the field of view of the detector 309 is variable. This configuration allows for altering the interrogation space, in near real time, to compensate for more or less concentrated samples, ensuring a low probability of two or more particles simultaneously being within an interrogation space. Similar alterations for two or more interrogation spaces, 370 and 371, may be performed.

In another embodiment, the interrogation space can be defined through the use of a calibration sample of known concentration that is passed through the capillary flow cell prior to the actual sample being tested. When only one single particle is detected at a time in the calibration sample as the sample is passing through the capillary flow cell, the depth of focus together with the diameter of the beam of the electromagnetic radiation source determines the size of the interrogation space in the capillary flow cell.

Physical constraints to the interrogation spaces can also be provided by a solid wall. In one embodiment, the wall is one or more of the walls of a flow cell 313 (FIG. 2A), when the sample fluid is contained within a capillary. In one embodiment, the cell is made of glass, but other substances transparent to light in the range of about 200 to about 1,000 nm or higher, such as quartz, fused silica, and organic materials such as Teflon, nylon, plastics, such as polyvinylchloride, polystyrene, and polyethylene, or any combination thereof, may be used without departing from the scope of the present invention. Although other cross-sectional shapes (e.g., rectangular, cylindrical) may be used without departing from the scope of the present invention, in one embodiment the capillary flow cell 313 has a square cross section. In another embodiment, the interrogation space may be defined at least in part by a channel (not shown) etched into a chip (not shown). Similar considerations apply to embodiments in which two interrogation spaces are used (370 and 371 in FIG. 2B).

The interrogation space is bathed in a fluid. In one embodiment, the fluid is aqueous. In other embodiments, the fluid is non-aqueous or a combination of aqueous and non-aqueous fluids. In addition the fluid may contain agents to adjust pH, ionic composition, or sieving agents, such as soluble macroparticles or polymers or gels. It is contemplated that valves or other devices may be present between the interrogation spaces to temporarily disrupt the fluid connection. Interrogation spaces temporarily disrupted are considered to be connected by fluid.

In another embodiment of the invention, an interrogation space is the single interrogation space present within the flow cell 313 which is constrained by the size of a laminar flow of the sample material within a diluent volume, also called sheath flow. In these and other embodiments, the interrogation space can be defined by sheath flow alone or in combination with the dimensions of the illumination source or the field of view of the detector. Sheath flow can be configured in numerous ways, including, but not limited to: the sample material is the interior material in a concentric laminar flow, with the diluent volume in the exterior; the diluent volume is on one side of the sample volume; the diluent volume is on two sides of the sample material; the diluent volume is on multiple sides of the sample material, but not enclosing the sample material completely; the diluent volume completely surrounds the sample material; the diluent volume completely surrounds the sample material concentrically; the sample material is the interior material in a discontinuous series of drops and the diluent volume completely surrounds each drop of sample material.

In some embodiments, single molecule detectors of the invention comprise no more than one interrogation space. In some embodiments, multiple interrogation spaces are used. Multiple interrogation spaces have been previously disclosed and are incorporated by reference from U.S. patent application Ser. No. 11/048,660. One skilled in the art will recognize that in some cases the analyzer will contain 2, 3, 4, 5, 6 or more distinct interrogation spaces.

3. Motive Force

In one embodiment of the analyzer system, the particles are moved through the interrogation space by a motive force. In some embodiments, the motive force for moving particles is pressure. In some embodiments, the pressure is supplied by a pump, an air pressure source, a vacuum source, a centrifuge, or a combination thereof. In some embodiments, the motive force for moving particles is an electrokinetic force. The use of an electrokinetic force as a motive force has been previously disclosed in a prior application and is incorporated by reference from U.S. patent application Ser. No. 11/048,660.

In one embodiment, pressure can be used as a motive force to move particles through the interrogation space of the capillary flow cell. In a further embodiment, pressure is supplied to move the sample by means of a pump. Suitable pumps are known in the art. In one embodiment, pumps manufactured for HPLC applications, such as those made by Scivax, Inc. can be used as a motive force. In other embodiments, pumps manufactured for microfluidics applications can be used when smaller volumes of sample are being pumped. Such pumps are described in U.S. Pat. Nos. 5,094,594, 5,730,187, 6,033,628, and 6,533,553, which discloses devices which can pump fluid volumes in the nanoliter or picoliter range. Preferably all materials within the pump that come into contact with sample are made of highly inert materials, e.g., polyetheretherketone (PEEK), fused silica, or sapphire.

A motive force is necessary to move the sample through the capillary flow cell to push the sample through the interrogation space for analysis. A motive force is also required to push a flushing sample through the capillary flow cell after the sample has been passed through. A motive force is also required to push the sample back out into a sample recovery vessel, when sample recovery is employed. Standard pumps come in a variety of sizes, and the proper size may be chosen to suit the anticipated sample size and flow requirements. In some embodiments, separate pumps are used for sample analysis and for flushing of the system. The analysis pump may have a capacity of approximately 0.000001 mL to approximately 10 mL, or approximately 0.001 mL to approximately 1 mL, or approximately 0.01 mL to approximately 0.2 mL, or approximately 0.005, 0.01, 0.05, 0.1, or 0.5 mL. Flush pumps may be of larger capacity than analysis pumps. Flush pumps may have a volume of about 0.01 mL to about 20 mL, or about 0.1 mL to about 10 mL, or about 0.1 mL to about 2 mL, or about or about 0.05, 0.1, 0.5, 1, 5, or 10 mL. These pump sizes are illustrative only, and those of skill in the art will appreciate that the suitable pump size may be chosen according to the application, sample size, viscosity of fluid to be pumped, tubing dimensions, rate of flow, temperature, and other factors well known in the art. In some embodiments, pumps of the system are driven by stepper motors, which are easy to control very accurately with a microprocessor.

In preferred embodiments, the flush and analysis pumps are used in series, with special check valves to control the direction of flow. The plumbing is designed so that when the analysis pump draws up the maximum sample, the sample does not reach the pump itself. This is accomplished by choosing the ID and length of the tubing between the analysis pump and the analysis capillary such that the tubing volume is greater than the stroke volume of the analysis pump.

4. Detectors

In one embodiment, light (e.g., light in the ultra-violet, visible or infrared range) emitted by a fluorescent label after exposure to electromagnetic radiation is detected. The detector 309 (FIG. 1A), or detectors (364, 365, FIG. 1B), is capable of capturing the amplitude and duration of photon bursts from a fluorescent label-moiety complex, and further converting the amplitude and duration of the photon burst to electrical signals. Detection devices such as CCD cameras, video input module cameras, and Streak cameras can be used to produce images with contiguous signals. In another embodiment, devices such as a bolometer, a photodiode, a photodiode array, avalanche photodiodes, and photomultipliers which produce sequential signals may be used. Any combination of the aforementioned detectors may also be used. In one embodiment, avalanche photodiodes are used for detecting photons.

Using specific optics between an interrogation space 314 (FIG. 2A) and its corresponding detector 309 (FIG. 1A), several distinct characteristics of the emitted electromagnetic radiation can be detected including: emission wavelength, emission intensity, burst size, burst duration, and fluorescence polarization. In some embodiments, the detector 309 is a photodiode that is used in reverse bias. A photodiode set in reverse bias usually has an extremely high resistance. This resistance is reduced when light of an appropriate frequency shines on the P/N junction. Hence, a reverse biased diode can be used as a detector by monitoring the current running through it. Circuits based on this effect are more sensitive to light than ones based on zero bias.

In one embodiment of the analyzer system, the photodiode can be an avalanche photodiode, which can be operated with much higher reverse bias than conventional photodiodes, thus allowing each photon-generated carrier to be multiplied by avalanche breakdown, resulting in internal gain within the photodiode, which increases the effective responsiveness (sensitivity) of the device. The choice of photodiode is determined by the energy or emission wavelength emitted by the fluorescently labeled particle. In some embodiments, the photodiode is a silicon photodiode that detects energy in the range of 190-1100 nm; in another embodiment the photodiode is a germanium photodiode that detects energy in the range of 800-1700 nm; in another embodiment the photodiode is an indium gallium arsenide photodiode that detects energy in the range of 800-2600 nm; and in yet other embodiments, the photodiode is a lead sulfide photodiode that detects energy in the range of between less than 1000 nm to 3500 nm. In some embodiments, the avalanche photodiode is a single-photon detector designed to detect energy in the 400 nm to 1100 nm wavelength range. Single photon detectors are commercially available (for example Perkin Elmer, Wellesley, Mass.).

In some embodiments the detector is an avalanche photodiode detector that detects energy between 300 nm and 1700 nm. In one embodiment, silicon avalanche photodiodes can be used to detect wavelengths between 300 nm and 1100 nm. Indium gallium arsenic photodiodes can be used to detect wavelengths between 900 nm and 1700 nm. In some embodiments, an analyzer system can comprise at least one detector; in other embodiments, the analyzer system can comprise at least two detectors, and each detector can be chosen and configured to detect light energy at a specific wavelength range. For example, two separate detectors can be used to detect particles that have been tagged with different labels, which upon excitation with an electromagnetic radiation source, will emit photons with energy in different spectra. In one embodiment, an analyzer system can comprise a first detector that can detect fluorescent energy in the range of 450-700 nm such as that emitted by a green dye (e.g. Alexa 546); and a second detector that can detect fluorescent energy in the range of 620-780 nm such as that emitted by a far-red dye (e.g. Alexa 647). Detectors for detecting fluorescent energy in the range of 400-600 nm such as that emitted by blue dyes (e.g. Hoechst 33342), and for detecting energy in the range of 560-700 nm such as that emitted by red dyes (Alexa 546 and Cy3) can also be used.

A system comprising two or more detectors can be used to detect individual particles that are each tagged with two or more labels that emit light in different spectra. For example, two different detectors can detect an antibody that has been tagged with two different dye labels. Alternatively, an analyzer system comprising two detectors can be used to detect particles of different types, each type being tagged with a different dye molecules, or with a mixture of two or more dye molecules. For example, two different detectors can be used to detect two different types of antibodies that recognize two different proteins, each type being tagged with a different dye label or with a mixture of two or more dye label molecules. By varying the proportion of the two or more dye label molecules, two or more different particle types can be individually detected using two detectors. It is understood that three or more detectors can be used without departing from the scope of the invention.

It should be understood by one skilled in the art that one or more detectors can be configured at each interrogation space, whether one or more interrogation spaces are defined within a flow cell, and that each detector may be configured to detect any of the characteristics of the emitted electromagnetic radiation listed above. The use of multiple detectors, e.g., for multiple interrogation spaces, has been previously disclosed in a prior application and is incorporated by reference here from U.S. patent application Ser. No. 11/048,660. Once a particle is labeled to render it detectable (or if the particle possesses an intrinsic characteristic rendering it detectable), any suitable detection mechanism known in the art may be used without departing from the scope of the present invention, for example a CCD camera, a video input module camera, a Streak camera, a bolometer, a photodiode, a photodiode array, avalanche photodiodes, and photomultipliers producing sequential signals, and combinations thereof. Different characteristics of the electromagnetic radiation may be detected including: emission wavelength, emission intensity, burst size, burst duration, fluorescence polarization, and any combination thereof.

C. Sampling System

In a further embodiment, the analyzer system may include a sampling system to prepare the sample for introduction into the analyzer system. The sampling system included is capable of automatically sampling a plurality of samples and providing a fluid communication between a sample container and a first interrogation space.

In some embodiments, the analyzer system of the invention includes a sampling system for introducing an aliquot of a sample into the single particle analyzer for analysis. Any mechanism that can introduce a sample may be used. Samples can be drawn up using either a vacuum suction created by a pump or by pressure applied to the sample that would push liquid into the tube, or by any other mechanism that serves to introduce the sample into the sampling tube. Generally, but not necessarily, the sampling system introduces a sample of known sample volume into the single particle analyzer; in some embodiments where the presence or absence of a particle or particles is detected, precise knowledge of the sample size is not critical. In preferred embodiments the sampling system provides automated sampling for a single sample or a plurality of samples. In embodiments where a sample of known volume is introduced into the system, the sampling system provides a sample for analysis of more than about 0.0001, 0.001, 0.01, 0.1, 1, 2, 5, 10, 20, 30, 40, 50, 60, 70, 80, 90, 100, 150, 200, 500, 1000, 1500, or 2000 μl. In some embodiments the sampling system provides a sample for analysis of less than about 2000, 1000, 500, 200, 100, 90, 80, 70, 60, 50, 40, 30, 20, 10, 5, 2, 1, 0.1, 0.01, or 0.001 μl. In some embodiments the sampling system provides a sample for analysis of between about 0.01 and about 1500 μl, or about 0.1 and about 1000 μl, or about 1 and about 500 μl, or about 1 and about 100 μl, or about 1 and about 50 μl, or about 1 and about 20 μl. In some embodiments, the sampling system provides a sample for analysis between about 5 μl and about 200 μl, or about 5 μl and about 100 μl, or about 5 μl and about 50 μl. In some embodiments, the sampling system provides a sample for analysis between about 10 μl and about 200 μl, or between about 10 μl and about 100 ul, or between about 10 μl and about 50 μl. In some embodiments, the sampling system provides a sample for analysis between about 0.5 μl and about 50 μl.

In some embodiments, the sampling system provides a sample size that can be varied from sample to sample. In these embodiments, the sample size may be any one of the sample sizes described herein, and may be changed with every sample, or with sets of samples, as desired.

Sample volume accuracy, and sample to sample volume precision of the sampling system, is required for the analysis at hand. In some embodiments, the precision of the sampling volume is determined by the pumps used, typically represented by a CV of less than about 50, 40, 30, 20, 10, 5, 4, 3, 2, 1, 0.5, 0.1, 0.05, or 0.01% of sample volume. In some embodiments, the sample to sample precision of the sampling system is represented by a CV of less than about 50, 40, 30, 20, 10, 5, 4, 3, 2, 1, 0.5, 0.1, 0.05, or 0.01%. In some embodiments, the intra-assay precision of the sampling system is represented by a CV of less than about 10, 5, 1, 0.5, or 0.1%. In some embodiments, the intra-assay precision of the sampling system shows a CV of less than about 5%. In some embodiments, the interassay precision of the sampling system is represented by a CV of less than about 10, 5, or 1%. In some embodiments, the interassay precision of the sampling system shows a CV of less than about 5%.

In some embodiments, the sampling system provides low sample carryover, advantageous in that an additional wash step is not required between samples. Thus, in some embodiments, sample carryover is less than about 1, 0.5, 0.1, 0.05, 0.04, 0.03, 0.02, 0.01, 0.005, or 0.001%. In some embodiments, sample carryover is less than about 0.02%. In some embodiments, sample carryover is less than about 0.01%.

In some embodiments the sampler provides a sample loop. In these embodiments, multiple samples are drawn into tubing sequentially and each is separated from the others by a “plug” of buffer. The samples typically are read one after the other with no flushing in between. Flushing is done once at the end of the loop. In embodiments where a buffer “plug” is used, the plug may be recovered ejecting the buffer plug into a separate well of a microtiter plate.

The sampling system may be adapted for use with standard assay equipment, for example, a 96-well microtiter plate, or, preferably, a 384-well plate. In some embodiments the system includes a 96 well plate positioner and a mechanism to dip the sample tube into and out of the wells, e.g., a mechanism providing movement along the X, Y, and Z axes. In some embodiments, the sampling system provides multiple sampling tubes from which samples may be stored and extracted from, when testing is commenced. In some embodiments, all samples from the multiple tubes are analyzed on one detector. In other embodiments, multiple single molecule detectors may be connected to the sample tubes. Samples may be prepared by steps that include operations performed on sample in the wells of the plate prior to sampling by the sampling system, or sample may be prepared within the analyzer system, or some combination of both.

D. Sample Preparation System

Sample preparation includes the steps necessary to prepare a raw sample for analysis. These steps can involve, by way of example, one or more steps of: separation steps such as centrifugation, filtration, distillation, chromatography; concentration, cell lysis, alteration of pH, addition of buffer, addition of diluents, addition of reagents, heating or cooling, addition of label, binding of label, cross-linking with illumination, separation of unbound label, inactivation and/or removal of interfering compounds and any other steps necessary for the sample to be prepared for analysis by the single particle analyzer. In some embodiments, blood is treated to separate out plasma or serum. Additional labeling, removal of unbound label, and/or dilution steps may also be performed on the serum or plasma sample.

In some embodiments, the analyzer system includes a sample preparation system that performs some or all of the processes needed to provide a sample ready for analysis by the single particle analyzer. This system may perform any or all of the steps listed above for sample preparation. In some embodiments, samples are partially processed by the sample preparation system of the analyzer system. Thus, in some embodiments, a sample may be partially processed outside the analyzer system first. For example, the sample may be centrifuged first. The sample may then be partially processed inside the analyzer by a sample preparation system. Processing inside the analyzer includes labeling the sample, mixing the sample with a buffer and other processing steps that will be known to one in the art. In some embodiments, a blood sample is processed outside the analyzer system to provide a serum or plasma sample, which is introduced into the analyzer system and further processed by a sample preparation system to label the particle or particles of interest and, optionally, to remove unbound label. In other embodiments preparation of the sample can include immunodepletion of the sample to remove particles that are not of interest or to remove particles that can interfere with sample analysis. In yet other embodiments, the sample can be depleted of particles that can interfere with the analysis of the sample. For example, sample preparation can include the depletion of heterophilic antibodies, which are known to interfere with immunoassays that use non-human antibodies to directly or indirectly detect a particle of interest. Similarly, other proteins that interfere with measurements of the particles of interest can be removed from the sample using antibodies that recognize the interfering proteins.

In some embodiments, the sample can be subjected to solid phase extraction prior to being assayed and analyzed. For example, a serum sample that is assayed for cAMP can first be subjected to solid phase extraction using a c18 column to which it binds. Other proteins such as proteases, lipases and phosphatases are washed from the column, and the cAMP is eluted essentially free of proteins that can degrade or interfere with measurements of cAMP. Solid phase extraction can be used to remove the basic matrix of a sample, which can diminish the sensitivity of the assay. In yet other embodiments, the particles of interest present in a sample may be concentrated by drying or lyophilizing a sample and solubilizing the particles in a smaller volume than that of the original sample. For example, a sample of exhaled breath condensate (EBC) can be dried and resuspended in a small volume of a suitable buffer solution to enhance the detection of the particle of interest.

In some embodiments the analyzer system provides a sample preparation system that provides complete preparation of the sample to be analyzed on the system, such as complete preparation of a blood sample, a saliva sample, a urine sample, a cerebrospinal fluid sample, a lymph sample, a BAL sample, an exhaled breath condensate sample (EBC), a biopsy sample, a forensic sample, a bioterrorism sample, or any other suitable sample and the like. In some embodiments the analyzer system provides a sample preparation system that provides some or all of the sample preparation. In some embodiments, the initial sample is a blood sample that is further processed by the analyzer system. In some embodiments, the sample is a serum or plasma sample that is further processed by the analyzer system. The serum or plasma sample may be further processed by, e.g., contacting with a label that binds to a particle or particles of interest; the sample may then be used with or without removal of unbound label.

In some embodiments, sample preparation is performed, either outside the analysis system or in the sample preparation component of the analysis system, on one or more microtiter plates, such as a 96-well plate. Reservoirs of reagents, buffers, and the like can be in intermittent fluid communication with the wells of the plate by means of tubing or other appropriate structures, as are well-known in the art. Samples may be prepared separately in 96 well plates or tubes. Sample isolation, label binding and, if necessary, label separation steps may be done on one plate. In some embodiments, prepared particles are then released from the plate and samples are moved into tubes for sampling into the sample analysis system. In some embodiments, all steps of the preparation of the sample are done on one plate and the analysis system acquires sample directly from the plate. Although this embodiment is described in terms of a 96-well plate, it will be appreciated that any vessel for containing one or more samples and suitable for preparation of sample may be used. For example, standard microtiter plates of 384 or 1536 wells may be used. More generally, in some embodiments, the sample preparation system is capable of holding and preparing more than about 5, 10, 20, 30, 40, 50, 60, 70, 80, 90, 100, 200, 300, 500, 1000, 5000, or 10,000 samples. In some embodiments, multiple samples may be sampled for analysis in multiple analyzer systems. Thus, in some embodiments, 2 samples, or more than about 2, 3, 4, 5, 7, 10, 15 20, 50, or 100 samples are sampled from the sample preparation system and run in parallel on multiple sample analyzer systems.

Microfluidics systems may also be used for sample preparation and as sample preparation systems that are part of analyzer systems, especially for samples suspected of containing concentrations of particles high enough that detection requires smaller samples. Principles and techniques of microfluidic manipulation are known in the art. See, e.g., U.S. Pat. Nos. 4,979,824; 5,770,029; 5,755,942; 5,746,901; 5,681,751; 5,658,413; 5,653,939; 5,653,859; 5,645,702; 5,605,662; 5,571,410; 5,543,838; 5,480,614, 5,716,825; 5,603,351; 5,858,195; 5,863,801; 5,955,028; 5,989,402; 6,041,515; 6,071,478; 6,355,420; 6,495,104; 6,386,219; 6,606,609; 6,802,342; 6,749,734; 6,623,613; 6,554,744; 6,361,671; 6,143,152; 6,132,580; 5,274,240; 6,689,323; 6,783,992; 6,537,437; 6,599,436; 6,811,668 and published PCT patent application no. WO9955461(A1). Samples may be prepared in series or in parallel, for use in a single or multiple analyzer systems.

Preferably, the sample comprises a buffer. The buffer can be mixed with the sample outside the analyzer system, or it can be provided by the sample preparation mechanism. While any suitable buffer can be used, the preferable buffer has low fluorescence background, is inert to the detectably labeled particle, can maintain the working pH and, in embodiments wherein the motive force is electrokinetic, has suitable ionic strength for electrophoresis. The buffer concentration can be any suitable concentration, such as in the range from about 1 to about 200 mM. Any buffer system may be used as long as it provides for solubility, function, and delectability of the molecules of interest. Preferably, for application using pumping, the buffer is selected from the group consisting of phosphate, glycine, acetate, citrate, acidulate, carbonate/bicarbonate, imidazole, triethanolamine, glycine amide, borate, MES, Bis-Tris, ADA, aces, PIPES, MOPSO, Bis-Tris Propane, BES, MOPS, TES, HEPES, DIPSO, MOBS, TAPSO, Trizma, HEPPSO, POPSO, TEA, EPPS, Tricine, Gly-Gly, Bicine, HEPBS, TAPS, AMPD, TABS, AMPSO, CHES, CAPSO, AMP, CAPS, and CABS. The buffer can also be selected from the group consisting of Gly-Gly, bicine, tricine, 2-morpholine ethanesulfonic acid (MES), 4-morpholine propanesulfonic acid (MOPS) and 2-amino-2-methyl-1-propanol hydrochloride (AMP). A useful buffer is 2 mM Tris/borate at pH 8. 1, but Tris/glycine and Tris/HCl are also acceptable. Other buffers are as described herein.

Buffers useful for electrophoresis are disclosed in a prior application and are incorporated by reference herein from U.S. patent application Ser. No. 11/048,660.

E. Sample Recovery

One highly useful feature of embodiments of the analyzers and analysis systems of the invention is that the sample can be analyzed without consuming it. This can be especially important when sample materials are limited. Recovering the sample also allows one to do other analyses or reanalyze it. The advantages of this feature for applications where sample size is limited and/or where the ability to reanalyze the sample is desirable, e.g., forensic, drug screening, and clinical diagnostic applications, will be apparent to those of skill in the art.

Thus, in some embodiments, the analyzer system of the invention further provides a sample recovery system for sample recovery after analysis. In these embodiments, the system includes mechanisms and methods by which the sample is drawn into the analyzer, analyzed and then returned, e.g., by the same path, to the sample holder, e.g., the sample tube. Because no sample is destroyed and because it does not enter any of the valves or other tubing, it remains uncontaminated. In addition, because all the materials in the sample path are highly inert, e.g., PEEK, fused silica, or sapphire, there is little contamination from the sample path. The use of the stepper motor controlled pumps (particularly the analysis pump) allows precise control of the volumes drawn up and pushed back out. This allows complete or nearly complete recovery of the sample with little if any dilution by the flush buffer. Thus, in some embodiments, more than about 50%, 60%, 70%, 80%, 85%, 90%, 95%, 96%, 97%, 98%, 99%, 99.5%, or 99.9% of the sample is recovered after analysis. In some embodiments, the recovered sample is undiluted. In some embodiments, the recovered sample is diluted less than about 1.5-fold, 1.4-fold, 1.3-fold, 1.2-fold, 1.1-fold, 1.05-fold, 1.01-fold, 1.005-fold, or 1.001-fold.

For sampling and/or sample recovery, any mechanism for transporting a liquid sample from a sample vessel to the analyzer may be used. In some embodiments the inlet end of the analysis capillary has attached a short length of tubing, e.g., PEEK tubing that can be dipped into a sample container, e.g. a test tube or sample well, or can be held above a waste container. When flushing, to clean the previous sample from the apparatus, this tube is positioned above the waste container to catch the flush waste. When drawing a sample in, the tube is put into the sample well or test tube. Typically the sample is drawn in quickly, and then pushed out slowly while observing particles within the sample. Alternatively, in some embodiments, the sample is drawn in slowly during at least part of the draw-in cycle; the sample may be analyzed while being slowly drawn in. This can be followed by a quick return of the sample and a quick flush. In some embodiments, the sample may be analyzed both on the inward (draw-in) and outward (pull out) cycle, which improves counting statistics, e.g., of small and dilute samples, as well as confirming results, and the like. If it is desired to save the sample, it can be pushed back out into the same sample well it came from, or to another. If saving the sample is not desired, the tubing is positioned over the waste container.

### VI. Methods Using Highly Sensitive Analysis of Prostate Specific Antigen

The methods of the present invention make possible measurement of PSA levels at concentrations far lower than previously measured. Although PSA is an accepted marker for cancer, its usefulness has been limited by the fact that, with current methods of analysis, it is only detectable after a certain amount of PSA has been released into the blood, because of the lack of sensitivity of current methods. PSA levels can be measured and monitored for the presence of cancer or a tumor or the recurrence of a tumor after cancer has gone into remission.

In some embodiments, the invention provides a method for determining a diagnosis, prognosis, or method of treatment in an individual by i) determining a concentration of PSA in a sample or determining the concentrations of PSA in a series of samples from the individual, where the concentration is determined by a PSA assay with a limit of detection for the PSA in said sample of less than about 150, 100, 80, 60, 40, 20, 10, 5, 1, 0.5, 0.1. or 0.05 pg/ml, e.g., less than about 100 pg/ml; and ii) determining a diagnosis, prognosis, or method of treatment in said individual, based on the concentration in the sample, or on the concentrations in the series of samples. The method of determining the concentration of PSA includes any suitable method with the requisite sensitivity, e.g., the methods descried herein. In some embodiments, the methods utilize a method of determining a concentration of PSA in the sample where the method comprises detecting single molecules of PSA, or complexes or fragments thereof.

In some embodiments, the threshold concentration of PSA is determined by analyzing samples, e.g., blood, serum, or plasma samples, from an apparently healthy population for PSA, and determining the level at which 80, 90, 95, 96, 97, 98, 99, 99.5, or 99.9% of the population fall below that level (concentration). This value is the threshold value. In women, or in men with prostate resection, there are already low levels of PSA present. Therefore, any measure of PSA about this threshold is indicative of a higher probability of cancer. In some embodiments, the threshold value is set at the 99^{th} percentile. This threshold value can be determined from a population in general or can be determined for a specific population. For example, in women normal PSA levels can be determined based on PSA levels detected from normal women who have yet to reach menopause. In some embodiments, the analyzing is performed using a method with a level of detection for the PSA of less than about 150, 100, 80, 60, 40, 20, 10, 5, 1, 0.5, 0.1. or 0.05 pg/ml, e.g., less than about 100 pg/ml.

In some embodiments, the invention provides a method for determining a diagnosis, prognosis, or method of treatment in an individual by comparing a value for a concentration of PSA in a sample from the individual with a normal value or a range of normal values for PSA, where the normal value or range of normal values is determined by a PSA assay with a limit of detection for the PSA in said sample of less than about 150, 100, 80, 60, 40, 20, 10, 5, 1, 0.5, 0.1. or 0.05 pg/ml, e.g., less than about 100 pg/ml; and ii) determining a diagnosis, prognosis, or method of treatment in said individual, based on comparison.

In some embodiments, the PSA is free PSA. In some embodiments, the PSA is PSA-ACT. In some embodiments, the PSA is PSA-A2M. The PSA can any suitable PSA complex. The method may use total PSA, e.g., free PSA and PSA-ACT and PSA-A2M, or PSA-ACT, or PSA-A2M, as described herein, in determining a diagnosis, prognosis, or method of treatment. In some embodiments, the method may use the concentration of free, complexed, or percentage of free PSA, or any comparison of these (e.g., a ratio), to determine a diagnosis, prognosis, or method of treatment.

A. Samples

The sample or series of samples may be any suitable sample; in some embodiments, the sample(s) will be blood, serum, or plasma. In some embodiments, the sample or series of samples are serum samples. The individual may be an animal, e.g., mammal, e.g., human.

A single sample may be taken, or a series of samples may be taken. If a series of samples is taken, they may be taken at any suitable interval, e.g., intervals of minutes, hours, days, weeks, months, or years. When an individual is followed for longer periods, sample intervals may be months or years. Diagnosis, prognosis, or method of treatment may be determined from a single sample, or from one or more of a series of samples, or from changes in the series of samples, e.g., an increase in concentration at a certain rate may indicate a severe condition whereas increase at a slower rate or no increase may indicate a relatively benign or less serious condition. The rate of change may be measured over the course of hours, days, weeks, months, or years. Rate of change in a given individual may, in some cases, be more relevant than an absolute value. In other settings, a rise in values over a period of days, weeks, months or years in an individual can indicate ongoing and worsening condition or recurrence of cancer.

In some embodiments, at least one sample is taken at or near the time the individual presents to a health professional with one or more symptoms indicative of a condition that may involve cancer or any other condition in which PSA levels are elevated. Settings in which an individual may present to a health care professional include, but are not limited to ambulatory, urgent care, critical care, intensive care, monitoring unit, inpatient, outpatient, physician office, medical clinic, emergency response setting, including an ambulance, and health screening settings. In some embodiments, one or more samples are taken from the individual and are assayed for PSA locally, i.e., at or near the setting at which the samples are taken. For example, an individual who presents at a hospital may have one or more samples taken that are assayed for PSA within the hospital. In some embodiments, one or more samples are taken from the individual and are assayed for PSA in a CLIA laboratory. In some embodiments, the individual displays one or more symptoms consistent with prostate cancer. Such symptoms include, but are not limited to, slowing or weakening of a urinary stream, increase in the frequency of urination, hematuria, or impotence, pain to the hips, spine, ribs, numbness in the legs or feet or loss of bladder or bowel control. In some embodiments, the individual displays one or more symptoms consistent with breast cancer. Such symptoms include, but are not limited to, nipple tenderness, a lump or thickening in the breast or underarm area, change in the size or shape of the breast, inverted nipple, scaly, red or swollen skin of the breast, areola, or nipple, ridges or pitting in the skin of the breast, areola or nipple, or nipple discharge. In addition prostate cancer and breast cancer have other molecular markers that are detectable in the blood. The detection of these markers, in addition to PSA, may give a more definitive diagnosis of a cancerous condition. Other molecular markers for prostate cancer and breast cancer are given below.

B. Determination of Diagnosis, Prognosis, or Method of Treatment

In some embodiments, step ii) includes comparing said concentration or series of concentrations to a normal value for said concentration, comparing said concentration or series of concentrations to a predetermined threshold level, comparing said concentration or series of concentrations to a baseline value, or determining a rate of change of concentration for said series of concentrations.

In some embodiments, step ii) comprises comparing said concentration of PSA in said sample with a predetermined threshold concentration, and determining a diagnosis, prognosis, or method of treatment if the sample concentration is greater than the threshold level. The threshold concentration can be determined by, e.g., determining the 99th percentile concentration of PSA in a group of individuals, and setting said threshold concentration at said 99th percentile concentration. An example of this is given in Examples.

Normal values, threshold values, rates of change, ratios of values, and other useful diagnostic and prognostic indicators may be established by methods well-known in the art. For example, these values may be determined by comparing samples from a case population and a control population, where the case population exhibits the biological state for which diagnosis, prognosis, or method of treatment is desired, and the control population does not exhibit the biological state. Another example is following the level of PSA of an individual over time. In this case, a patient is tested on consecutive weeks, months, or years, which will further serve as a reference for that individual. In some embodiments, a longitudinal study may be done, e.g., the case population may be a subset of the control population that, over time, exhibits the biological state. It will be appreciated that data from a plurality of studies may be used to determine a consensus value or range of values for normal, and for prognostic or diagnostic levels.

In developing a diagnostic or prognostic test, data for one or more potential markers may be obtained from a group of subjects. The group of subjects is divided into at least two sets, and preferably the first set and the second set each have an approximately equal number of subjects. The first set includes subjects who have been confirmed as having cancer or, more generally, being in a first condition state. For example, this first set of patients may be those that have recently had a disease incidence, or may be those having a specific type of cancer, such as prostate cancer. The confirmation of the condition state may be made through a more rigorous testing such as a digital rectal exam (DRE), transrectal ultrasound (TRUS), biopsy, or a mammogram. Hereinafter, subjects in this first set will be referred to as “diseased”. The second set of subjects is simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects. In still another alternative, this second set may represent those at a different time point from disease incidence. Preferably, data for the same set of markers is available for each patient. This set of markers may include all candidate markers which may be suspected as being relevant to the detection of a particular disease or condition. Actual known relevance is not required. Embodiments of the compositions, methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

1. Prostate Cancer

The level of PSA in a sample is used to detect prostate cancer in men. The National Cancer Institute branch of the NIH has indicated that 0 to 2.5 ng/ml is a low level range of PSA. The higher a man's PSA level, the more likely it is that cancer is present. PSA levels above 4 ng/ml is usually considered an indicator of prostate cancer, but in some cases, men with PSA levels below 4 ng/ml have been diagnosed with prostate cancer. Screening for prostate cancer involves measuring the percentage of free PSA in a sample (the ratio of free PSA to the total PSA). The percentage of free PSA will be lower in men who have prostate cancer than in men who do not. A biopsy is recommended for men with a percentage of free PSA less than 10% and is encouraged for men with a percentage of free PSA of less than 25%. Another option might be to measure only complexed or non-free PSA instead of the total and the free PSA. Another measurement regarding PSA is how fast the PSA levels rise over time. The faster the rise in PSA levels the more cause for concern. For this method, it would be good to detect low levels of PSA. In addition, the low level of detection of PSA is advantageous because of the small percentage of the male population who have low levels of PSA but who are nonetheless still diagnosed with prostate cancer. Thus, the invention disclosed provides methods for diagnosis, prognosis, or methods of treatment based on the highly sensitive detection of PSA in individuals.

For treatment of prostate cancer, a measure of the level of PSA in a sample is also advantageous in the diagnosis of recurrence of prostate cancer in an individual who has undergone surgical resection. After surgical resection of the prostate, PSA levels in the blood are significantly reduced to almost undetectable levels. Elevation in the PSA level from the low levels of PSA after surgical resection may be an indicator of recurrence of prostate cancer after surgical resection. In this case, an individual who has undergone surgical resection may be monitored for changes in the level of PSA. After surgical resection, a measure of the level of PSA in a sample taken from an individual can be made. This level of PSA measured after surgical resection can be set as the threshold level of PSA for that individual. The levels of PSA can then be monitored over time by taking samples from an individual at different time periods after surgical resection. Further, clinical trials can establish at what percentage over a threshold level of PSA the level of PSA is an indicator of recurrence of prostate cancer. In some cases, the percentage is about 5% over the threshold level. In some cases the percentage is about 10% over the threshold level. In some cases, the percentage is about 15% over the threshold level.

In some embodiments, at least one sample is taken before or after a digital rectal exam (DRE) or a transrectal ultrasound (TRUS). Elevated levels of PSA may be an indication that the patient has prostate cancer. Also, elevations in PSA levels can be an indicator that a patient is no longer in remission. Post surgical resection, PSA levels currently go undetectable, since current tests have a lower limit of quantitation of 100 pg/ml. One method is to measure a PSA level post surgical resection wherein the value of the PSA decreases to levels less than approximately 100 pg/ml, or approximately less than 50 pg/ml, or approximately less than 10 pg/ml, or approximately less than 1 pg/ml, or approximately less than 0.5 pg/ml. This value becomes the threshold level of PSA for that individual. The PSA levels of the patient can then be monitored over time by measuring the PSA levels in a series of samples. An increase in the level of PSA a predetermined percentage above the threshold level is an indication of recurrence of prostate cancer after surgical resection. Other comparisons may be done as well, such as comparisons of any of the samples to normal or threshold levels, or determination of a rate of change in the concentration of PSA in the samples, all of which may yield useful information regarding presence of cancers, as well as other conditions as described herein.

Thus, the invention provides a method of diagnosing, predicting, and/or preventing or treating prostate cancer in an individual by assaying a sample from the individual, e.g., a blood sample, plasma sample, and/or serum sample, for PSA, e.g., free PSA, and detecting a concentration of PSA in the sample at a limit of detection of less than about 150, 100, 80, 60, 40, 20, 10, 5, 1, 0.5, 0.1. or 0.05 pg/ml, e.g., less than about 100 pg/ml, wherein the concentration of PSA in the sample indicates or predicts prostate cancer. The PSA may be free PSA, PSA-ACT, or PSA-A2M, and may be total PSA or a measure of a particular form, e.g., free, complexed, or fragment; in some embodiments, a ratio of one or more forms of the PSA is used, as described herein. In some embodiments, PSA-ACT is measured in the sample or series of samples. In some embodiments, PSA-A2M is measured in the sample or series of samples. In some embodiments, total PSA is measured in the sample or series of samples. In some embodiments, the PSA level is determined at or near the time the individual presents to a health professional with symptoms indicative of prostate cancer. Such symptoms include, but are not limited to, slowing or weakening of a urinary stream, increase in the frequency of urination, hematuria, or impotence, pain to the hips, spine, ribs, numbness in the legs or feet or loss of bladder or bowel control.

In some embodiments, a series of measurements is taken, and an elevated level of PSA concentration in the samples indicates, predicts, or provides a basis for prognosis of prostate cancer. In some embodiments, a level of over 50%, over 100%, over 150%, over 200%, over 250%, over 300%, over 400%, or over 500% of baseline indicates, predicts, or provides a basis for prognosis of prostate cancer or recurrence of prostate cancer. In some embodiments, a PSA level of over about 0.25, 0.3, 0.35, 0.4 ng/ml in a single sample indicates, predicts, or provides a basis for prognosis of prostate cancer, regardless of baseline levels, if obtained. In some embodiments, a PSA level of about 0.25-4, or about 0.25-10, or about 10-15, or about 10-20, about 20-30, or about 20-40 ng/ml indicates, predicts, or provides a basis for prognosis of prostate cancer.

In some embodiments, diagnosis or prognosis includes stratification for the individual, based on PSA concentration in the sample or series of samples. Such stratification may be based on the concentration of PSA in single samples, ratios of different forms of PSA, absolute values for different forms of PSA, rate of change in concentration for PSA or for one or more forms of PSA in a series of samples, change in ratios of different forms of PSA over time in a series of samples, and any other information based at least in part on PSA concentration in the sample or series of samples. Stratification may be based on values obtained from populations of normal and diseased subjects, as described herein. Appropriate treatment may also be determined based on the stratification of the individual.

In some embodiments, concentration of PSA is determined in combination with one or more other markers, e.g. p53, and the concentrations of each marker are considered in determining the diagnosis, prognosis, or method of treatment. Other clinical indications typically will also be taken into account, e.g., DRE results, symptoms, history, and the like, as will be apparent to those of skill in the art. Appropriate algorithms for diagnosis, prognosis, or treatment may be constructed based on the combinations of such markers and clinical indications in combination with PSA levels.

Markers useful in combination with PSA in the methods of the invention include but are not limited to prostate specific membrane antigen (PSMA), KIAA 18, KIAA 96, prostate carcinoma tumor antigen-1 (PCTA-1), prostate secretory protein (PSP), prostate acid phosphatase (PAP), human glandular kallekrein 2 (HK-2), prostate stem cell antigen (PSCA), PTI-1, CLAR1 (U.S. Pat. No. 6,361,948), PG1, BPC-1, prostate-specific transglutaminase, cytokeratin 15, semenogelin II, NAALADase, PD-41, p53, TCSF (U.S. Pat. No. 5,856,112), p300, actin, EGFR, and HER-2/neuprotein. Other markers of inflammation may be detected, and include combinations of Il-8, IL-1β, IL6, IL10, TNF, and IL-12p70, as well as other cytokines or markers that will be apparent to those of skill in the art.

In some embodiments, PSA, alone or in combination with other markers or clinical signs, measured as described herein, is used to determine whether the tumor is no longer in remission. In some embodiments PSA, alone or in combination with other markers or clinical signs, measured as described herein, is used to determine the extent of the tumor. In the latter case, percent of free PSA may be compared to total PSA; the smaller the percentage of free PSA, the more likely the presence of prostate cancer.

PSA is also known as human kallikrein 3 (hk3), and is a member of the kallikrein (KLK) family. Elevated levels of kallikreins have been shown to be indicators of various types of cancerous conditions. Shaw et al, Clinical Chemistry 2007; 53(8) 1423-32. There are 15 different types of KLKs (KLK1 through KLK15) and each can be found in various tissues or fluid samples of a human. Conditions in which elevated levels of kallikreins can be used as an indicator include conditions that affect the pancreas, salivary glands, colon, small intestine, esophagus, kidney, lymph node, prostate gland, stomach, thyroid, ureter, vagina, adrenal gland, skin, breast, brain, spinal cord, heart, tonsils, liver, cervix, fallopian tube, lung, thymus, trachea, testis, bone marrow, bone, and cartilage. As in diagnosis of prostate cancer, a specific kind of KLK, can be measured in a sample taken from an individual and compared to a threshold level for that KLK. The threshold level of KLK can be the level of that specific KLK found in a reference population. A level of a specific KLK above a percentage of the threshold level of KLK may be considered an indication of the presence of a condition associated with that specific KLK. For example, KLK3 (also known as hk3) is an indicator of prostate health. A measure of KLK3 in addition to PSA may offer diagnostic value to the diagnosis of prostate cancer. KLK3 values for a reference population may be compared to the KLK3 values of an individual and if this value is higher than 10%, 12%, 15%, or higher of the threshold value, this would be an indicator of a condition affecting the prostate.

2. Breast Cancer

PSA has been detected in women as well as in men. The normal range of PSA in men is believed to be from approximately 100-200 pg/ml to approximately 1000 pg/ml. The normal range of PSA in women has not been detectable due to the low levels of PSA in the sera of women. Because of the sensitivity of current systems/assays for detecting PSA (current assays have the limit of detection at 5-10 pg/ml and the lower limit of quantification is 50-100 pg/ml), the normal range of PSA has not been defined.

PSA is not exclusively synthesized by the human prostate gland, but it is also produced by the breast, ovary, liver, kidney, pancreas, lung, adrenal and parotid glands. PSA expression has been shown to be inversely associated with the presence of breast cancer. PSA has been found in tumor extracts, in milk of lactating women, and in nipple aspirates. It has been shown that PSA levels detected in nipple aspirates is inversely associated with the presence of breast cancer and PSA levels decreased in tumors with more advanced disease stage, larger tumor size and nodal involvement (Narita et. al, Folia Histochem Cytobiol. 2006; 44(3):165-72). It has also been shown that PSA better predicts disease involvement in pre-menopausal women. PSA has been shown to be present in benign breast tumors, normal breast tissue and in breast cancer. Very low levels of circulating PSA are detectable in female sera.

The low levels of PSA detectable in women may be used to determine whether or not a woman is developing breast cancer. In some embodiments, the level of PSA in a sample taken from an individual is measured. This value is compared with the value of PSA in a reference population. This reference population may include all women, or all men. It may include women who are pre-menopausal or post-menopausal. It may include a population of individuals based on race, or age, or nationality, family history of breast cancer, or any other demographic in which to categorize an individual. This is important for cases in which breast cancer is more prevalent in one group of individuals over another group of individuals. The level of PSA is then compared to the threshold level. In cases where PSA is used as an indicator of breast cancer, the lower the level of PSA in an individual compared to baseline, the higher the likelihood of advanced disease stage, larger tumor size and nodal involvement. Because PSA levels in nipple aspirates have been shown to be inversely associated with the presence of breast cancer a decrease in the value of PSA below threshold level may be an indication of breast cancer in a woman.

In some embodiments, at least one sample is taken before or after a mammogram. The levels of PSA may be an indication that the patient has breast cancer. Also, the PSA level can be an indicator that a patient's cancer is no longer in remission. Other comparisons may be done as well, such as comparisons of any of the samples to normal or threshold levels, or determination of a rate of change in the concentration of PSA in the samples, all of which may yield useful information regarding presence of cancers, as well as other conditions as described herein.

In determining a normal range of PSA for women, the threshold would be 99% of the distribution of levels detected in normal women. In some embodiments, the threshold concentration of PSA is determined by analyzing samples, e.g., blood, serum, or plasma samples, from an apparently healthy population for PSA, and determining the level at which about 80, 90, 95, 96, 97, 98, 99, 99.5, or 99.9% of the population fall below that level (concentration) The distribution of PSA in a reference normal population can be measured and then, for an individual, the increase or decrease in PSA levels as compared to the reference population could be determined. This method would take into account the different threshold levels of normal women depending on age populations, race, and other factors. Thus, the invention provides a method of diagnosing, predicting, and/or preventing or treating breast cancer in an individual by assaying a sample from the individual, e.g., a blood sample, plasma sample, and/or serum sample, for PSA, e.g., total PSA, and detecting a concentration of PSA in the sample at a limit of detection of less than about 150, 100, 80, 60, 40, 20, 10, 5, 1, 0.5, 0.1. or 0.05 pg/ml, e.g., less than about 100 pg/ml, wherein the concentration of PSA in the sample indicates or predicts breast cancer. The PSA may be free PSA, total PSA, PSA-ACT, or PSA-A2M, or a measure of a particular form of PSA, e.g., free, complexed, or fragment; in some embodiments, a ratio of one or more forms of the PSA is used, as described herein. In some embodiments, PSA-ACT is measured in the sample or series of samples. In some embodiments, PSA-A2M is measured in the sample or series of samples. In some embodiments, total PSA is measured in the sample or series of samples. In some embodiments, the PSA level is determined at or near the time the individual presents to a health professional with symptoms indicative of breast cancer. Such symptoms include, but are not limited to, nipple tenderness, a lump or thickening in the breast or underarm area, change in the size or shape of the breast, inverted nipple, scaly, red or swollen skin of the breast, areola, or nipple, ridges or pitting in the skin of the breast, areola or nipple, or nipple discharge.

In some embodiments, a series of measurements is taken, and an elevated level of PSA concentration in the samples indicates, predicts, or provides a basis for prognosis of breast cancer. In some embodiments, a level of about over 50%, over 100%, over 150%, over 200%, over 250%, over 300%, over 400%, or over 500% of baseline indicates, predicts, or provides a basis for prognosis of breast cancer or recurrence of breast cancer. In some embodiments, a PSA level of over about 0.5 ng/ml in a single sample indicates, predicts, or provides a basis for prognosis of breast cancer, regardless of baseline levels, if obtained. Using the invention described herein, PSA has been detected in normal female sera and plasma. In some embodiments, the PSA concentration in normal female sera and plasma is greater than about 4 pg/ml, or about 3-4 pg/ml, or about 2-3 pg/ml, or about 1-2 pg/ml, or about less than 1 pg/ml. In some embodiments, the PSA concentration in normal female sera and plasma is about 0-0.1 pg/ml, or about 0.1-0.2, or about 0.2-0.3, or about 0.3-0.4, or about 0.4-0.5, or about 0.5-0.6, or about 0.6-0.7, or about 0.7-0.8, or about 0.8-0.9, or about 0.9-1 pg/ml.

In some embodiments diagnosis or prognosis includes stratification for the individual, based on PSA concentration in the sample or series of samples. Such stratification may be based on the concentration of PSA in single samples, ratios of different forms of PSA, absolute values for different forms of PSA, rate of change in concentration for PSA or for one or more forms of PSA in a series of samples, change in ratios of different forms of PSA over time in a series of samples, and any other information based at least in part on PSA concentration in the sample or series of samples. Stratification may be based on values obtained from populations of normal and diseased subjects, as described herein. Appropriate treatment may also be determined based on the stratification of the individual.

In some embodiments, concentration of PSA is determined in combination with one or more other markers, e.g. estrogen, and the concentrations of each marker are considered in determining the diagnosis, prognosis, or method of treatment. Other clinical indications typically will also be taken into account, e.g., mammogram results, symptoms, history, and the like, as will be apparent to those of skill in the art. Appropriate algorithms for diagnosis, prognosis, or treatment may be constructed based on the combinations of such markers and clinical indications in combination with PSA levels.

Markers useful in combination with PSA in the methods of the invention include but are not limited to estrogen, epidermal growth factor (EGF), transforming growth factor (TGF), prostaglandin E2 (PGE2); estrogen-regulated proteins such as pS2; interleukins (eg., IL-10); S-100 protein; vimentin; epithelial membrane antigen; bel-2; CA15-3 (an aberrant form of polymorphic epithelial mucin (PEM)); CA 19-9; mucin core carbohydrates (eg., Tn antigen and Tn-like antigens); alpha-lactalbumin; lipid-associated sialic acid (LASA); galactose-N-acetylgalactosamine (Gal-GalNAC); GCDFP-15; Le(y)-related carbohydrate antigen; CA 125; urokinase-type plasminogen activator (uPA) and uPA related antigens and complexes (eg., LMW-uPA, HMW-uPA, uPA aminoterminal fragment (ATF), uPA receptor (uPAR) and complexes with inhibitors such as PA1-1 and PA1-2); beta-glucuronidase; CD31; CD44 splice variants; blood group antigens (eg., ABH, Lewis, and MN). Other markers of inflammation may be detected, and include combinations of Il-8, IL-1, IL6, IL10, TNF, and IL-12p70, as well as other cytokines or markers that will be apparent to those of skill in the art.

In some embodiments, PSA, alone or in combination with other markers or clinical signs, measured as described herein, is used to determine whether the tumor is no longer in remission. In some embodiments, PSA, alone or in combination with other markers or clinical signs, measured as described herein, is used to determine the extent of the tumor. In the latter case, percent of free PSA may be compared to total PSA; the smaller the percentage of free PSA, the more likely the presence of breast cancer.

3. Other Conditions

As previously mentioned, PSA is not exclusively synthesized by the human prostate gland, but it is also produced by the breast, ovary, liver, kidney, pancreas, lung, adrenal and parotid glands. This leads to other possibilities of PSA as an indicator of cancerous conditions. In women, increased levels of PSA could possibly be indicative of ovarian, liver, kidney, pancreatic, lung, adrenal, or parotid cancer. Also, in men who have had surgical resection of the prostate, increased PSA levels could also indicate these cancers.

C. Business Methods

The present invention relates to systems and methods (including business methods) for establishing markers of prostate specific antigen that can be used for diagnosing, prognosing, or determining a method of treatment of a biological state or a condition in an organism, preparing diagnostics based on such markers, and commercializing/marketing diagnostics and services utilizing such diagnostics. The biological state may be prostate cancer, or breast cancer, or other non-prostate cancer states as described herein.

In one embodiment, the business methods herein comprise: establishing one or more PSA markers using a method comprising: establishing a range of concentrations for said marker or markers in biological samples obtained from a first population by measuring the concentrations of the marker or markers in the biological samples by detecting single molecules of the marker or markers at a level of detection of less than about 150, 100, 80, 60, 40, 20, 10, 5, 1, 0.5, 0.1, or 0.05 pg/ml, e.g., less than about 100 pg/ml; and commercializing the one or more markers established in the above step, e.g., in a diagnostic product. The diagnostic product herein can include one or more antibodies that specifically binds to the PSA and a fluorescent moiety that is capable of emitting an average of at least about 200 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules.

In one embodiment, the business methods herein comprise: establishing a range of normal values for PSA using a system comprising: establishing a range of concentrations for said PSA in biological samples obtained from a first population by measuring the concentrations of the marker the biological samples by detecting single molecules of the marker at a level of detection less than about 150, 100, 80, 60, 40, 20, 10, 5, 1, 0.5, 0.1. or 0.05 pg/ml, e.g., less than about 100 pg/ml; and providing a diagnostic service to determine if an organism has or does not have a state or condition of interest, e.g., recurrence of prostate cancer or breast cancer, or other non-prostate cancer condition. A diagnostic service herein may be provided by a CLIA approved laboratory that is licensed under the business or the business itself. The diagnostic services herein can be provided directly to a health care provider, a health care insurer, or a patient. Thus the business methods herein can make revenue from selling e.g., diagnostic services or diagnostic products.

The business methods herein also contemplate providing diagnostic services to, for example, health care providers, insurers, patients, etc. The business herein can provide diagnostic services by either contracting out with a service lab or setting up a service lab (under Clinical Laboratory Improvement Amendment (CLIA) or other regulatory approval). Such service lab can then carry out the methods disclosed herein to identify if PSA is within a sample.

### VII. Compositions

The invention provides compositions useful in the detection and quantitation of prostate specific antigen. Compositions include binding partners to PSA that are labeled with suitable labels for detection by the methods of the invention, pairs of binding partners in which one or both of the binding partners are labeled with suitable labels for detection by the methods of the invention, solid supports to which capture binding partners are attached, in some embodiments also with detection binding partners.

Exemplary embodiments include a composition for the detection of PSA that includes a binding partner to the PSA attached to a fluorescent moiety, where the fluorescent moiety is capable of emitting an average of at least about 200 photons when simulated by a laser emitting light at the excitation wavelength of the moiety, where the laser is focused on a spot of not less than about 5 microns in diameter that contains the moiety, and wherein the total energy directed at the spot by the laser is no more than about 3 microJoules. In some embodiments, the binding partner includes an antibody to PSA. In some embodiments, the antibody is a polyclonal antibody. In some embodiments, the antibody is a monoclonal antibody. In some embodiments, the PSA is selected from the group consisting of free PSA and complexed PSA. In some embodiments, the complexed PSA is PSA-ACT. In some embodiments, complexed PSA is PSA-A2M. The fluorescent moiety may contain one or more molecules that comprises at least one substituted indolium ring system in which the substituent on the 3-carbon of the indolium ring contains a chemically reactive group or a conjugated substance. The label composition may include a fluorescent moiety that includes one or more dye molecules selected from the group consisting of AlexaFluor 488, 532, 647, 700, or 750. The label composition may include a fluorescent moiety that includes one or more dye molecules selected from the group consisting of AlexaFluor 488, 532, 700, or 750. The label composition may include a fluorescent moiety that includes one or more dye molecules that are AlexaFluor 488. The label composition may include a fluorescent moiety that includes one or more dye molecules that are AlexaFluor 555. The label composition may include a fluorescent moiety that includes one or more dye molecules that are AlexaFluor 610. The label composition may include a fluorescent moiety that includes one or more dye molecules that are AlexaFluor 647. The label composition may include a fluorescent moiety that includes one or more dye molecules that are AlexaFluor 680. The label composition may include a fluorescent moiety that includes one or more dye molecules that are AlexaFluor 700. The label composition may include a fluorescent moiety that includes one or more dye molecules that are AlexaFluor 750.

In some embodiments, the invention provides a composition that includes a set of standards for the determination of a concentration of a PSA, wherein at least one of the standards is at a concentration of total PSA less than about 150, 100, 50, 25, 10, 5, 1, 0.5, 0.1, or 0.05 pg/ml, e.g., less than about 100 pg/ml. In some embodiments, the invention provides a composition that includes a set of standards for the determination of a concentration of a total PSA, wherein at least one of the standards is at a concentration of total PSA less than about 150 pg/ml. In some embodiments, the invention provides a composition that includes a set of standards for the determination of a concentration of a total PSA, wherein at least one of the standards is at a concentration of total PSA less than about 50 pg/ml. In some embodiments, the invention provides a composition that includes a set of standards for the determination of a concentration of a total PSA, wherein at least one of the standards is at a concentration of total PSA less than about 10 pg/ml. In some embodiments, the invention provides a composition that includes a set of standards for the determination of a concentration of a total PSA, wherein at least one of the standards is at a concentration of total PSA less than about 5 pg/ml. In some embodiments, the invention provides a composition that includes a set of standards for the determination of a concentration of a total PSA, wherein at least one of the standards is at a concentration of total PSA less than about 1 pg/ml. In some embodiments, the invention provides a composition that includes a set of standards for the determination of a concentration of a total PSA, wherein at least one of the standards is at a concentration of total PSA less than about 0.5 pg/ml. In some embodiments, the invention provides a composition that includes a set of standards for the determination of a concentration of a total PSA, wherein at least one of the standards is at a concentration of total PSA less than about 0.1 pg/ml. In some embodiments, the invention provides a composition that includes a set of standards for the determination of a concentration of a total PSA, wherein at least one of the standards is at a concentration of total PSA less than about 0.05 pg/ml.

Other compositions of the invention are as described herein.

### VIII. Kits

The invention further provides kits. Kits of the invention include one or more compositions useful for the sensitive detection of prostate specific antigen, as described herein, in suitable packaging. In some embodiments kits of the invention provide labels, e.g., binding partner such as an antibody that is specific for prostate specific antigen, where the binding partner is attached to a fluorescent moiety. In some embodiments kits of the invention provide binding partner pairs, e.g., antibody pairs, that are specific for prostate specific antigen, where at least one of the binding partners is a label for prostate specific antigen, as described herein. In some embodiments, the binding partners, e.g., antibodies, are provided in separate containers. In some embodiments, the binding partners, e.g., antibodies, are provided in the same container. In some embodiments, one of the binding partners, e.g., antibodies, is immobilized on a solid support, e.g., a microtiter plate or a paramagnetic bead. In some of these embodiments, the other binding partner, e.g., antibody, is labeled with a fluorescent moiety as described herein.

Binding partners, e.g., antibodies, solid supports, and fluorescent labels for components of the kits may be any suitable such components as described herein.

The kits may additionally include reagents useful in the methods of the invention, e.g., buffers and other reagents used in binding reactions, washes, buffers or other reagents for preconditioning the instrument on which assays will be run, and elution buffers or other reagents for running samples through the instrument.

Kits may include one or more standards, e.g., standards for use in the assays of the invention, such as standards of highly purified, PSA, or various fragments, complexes, and the like, thereof. Kits may further include instructions.

### EXAMPLES

The following examples are offered by way of illustration and not by way of limiting the remaining disclosure. Unless otherwise specified, processing samples in the Examples were analyzed in single molecule detector (SMD) as described herein, with the following parameters: Laser: continuous wave gallium arsenite diode laser of wavelength 639 nm (Blue Sky Research, Milpitas, Calif.), focused to a spot size of approximately 2 microns (interrogation space of 0.004 pL as defined herein); flow rate=5 microliter/min through a fused silica capillary of 100 micron square ID and 300 micron square OD; non-confocal arrangement of lenses; focusing lens of 0.8 numerical aperture (Olympus); silicon avalanche photodiode detector (Perkin Elmer, Waltham, Mass.).

### Example 1

### Comparison of PSA Assay Platforms

Assay: The purpose of this assay was to compare the sensitivity and reliability of the PSA assay system.

Materials: the following materials were used in the procedure described below: Assay plate: Nunc Maxisorp, product 464718, 384 well, clear, passively coated with a monoclonal antibody, BiosPacific #8311 (1 mg/ml in citrate-phosphate-NaC buffer pH 6.0, with 0.1% sodium azide as a preservative, stored at +2 to +8° C. For the standard curve, human PSA antigen (BioSpecific #J63000190) was used. The diluent for the standard concentrations was human serum. The standard was diluted to 10 ug/ml, aliquoted and frozen to −80° C. Dilution of the standards was done in a 96 well, conical, polypropylene, (Nunc product #249944). The following buffers and solutions were used: (a) assay buffer: BBS with 1% BSA and 0.1% Triton X-100; (b) detection antibody (Ab): goat polyclonal antibody affinity purified (BioSpecific G126C), which was labeled with fluorescent dye AlexaFluor 647, and stored at 4° C.; detection antibody diluent: 50% assay buffer, 50% passive blocking solution; wash buffer: borate buffer saline (BBS) with Triton X-100 (1.0 M borate, 15.0 M sodium chloride, 10% Triton X-100, pH=8.3); elution buffer: BBS with 4M urea, 0.02% Triton X-100 and 0.001% BSA.

Procedure: total PSA standard and sample preparation and analysis:

The standard curve was prepared as follows: working standards were prepared (0-120 pg/ml) by serial dilutions of the stock of PSA into standard diluent or to achieve a range of PSA concentrations of between 1.2 pg/ml 4.3 μg/ml.

10 μl passive blocking solution and 10 μl of standard or of sample were added to each well. Standards were run in quadruplicate. The plate was sealed with Axyseal sealing film, centrifuged for 1 min at 3000 RPM, and incubated for 2 hours at 25° C. with shaking. The plate was washed five times, and centrifuged until rotor reached 3000 RPM in an inverted position over a paper towel. A 1 nM working dilution of detection antibody was prepared, and 20 μl detection antibody were added to each well. The plate was sealed and centrifuged, and the assay incubated for 1 hour at 25° C. with shaking. 30 μl elution buffer were added per well, the plate was sealed and the assay incubated for 1 hour at 25° C. The plate was either stored for up to 48 hours at 4° C. prior to analysis, or the sample was analyzed immediately.

A similar procedure was followed for standards run on ACS: Centaur assays and Beckman ACCESS assays.

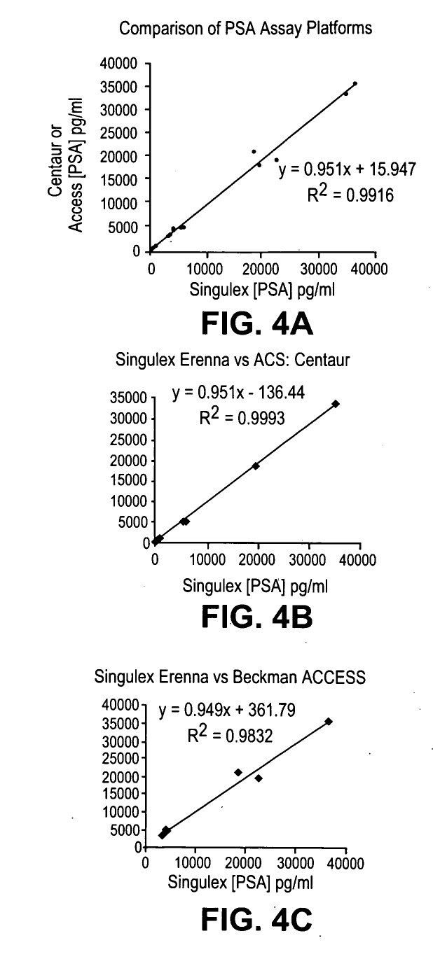

Results: Assays of PSA standards for all three assay detection systems showed similar detection sensitivity for PSA. The concentration of PSA for each assay method is shown in Table 3. Linearized standard curves comparing the different assays are shown in FIGS. 4A-4C. The R2 value comparing the Singulex PSA assay system and the ACS:Centaur or Beckman ACCESS PSA platforms is 0.9916. The R2 value for the Singulex Erenna system vs. ACS:Centaur and Beckman ACCESS PSA assays are 0.9993 and 0.9832, respectively. Singulex PSA assay system is as accurate as the current PSA detection systems on the market.

TABLE 3
Concentration of PSA Detected [pg/ml]

| Sample | | ACS: | Beckman |
|---|---|---|---|
| ID | Singulex | Centaur | ACCESS |
| 10300574 | 299 | 280 | |
| 10300579 | 737 | 760 | |
| 10300580 | 1007 | 820 | |
| 10300583 | 373 | 430 | |
| 10300755 | 425 | 560 | |
| 10569446 | 5508 | 5000 | |
| 10670330 | 3322 | | 3130 |
| 10670331 | 3606 | | 3450 |
| 10670333 | 4291 | | 4270 |
| 10670335 | 4067 | | 4620 |
| 11035092 | 19684 | 18200 | |
| 11047057 | 35271 | 33320 | |
| 11091414 | 36860 | | 35530 |
| 11091712 | 18690 | | 21020 |
| 11091745 | 22836 | | 19410 |

### Example 2

### Sandwich Assays for Biomarkers: Total PSA

Assay: The purpose of this assay was to detect the presence of total PSA in human serum. The assay format was a two-step sandwich immunoassay based on human PSA antigen and an affinity purified goat polyclonal detection antibody. Ten microliters of sample were required. The working range of the assay is 0-100 pg/ml with a typical analytical limit of detection of 0.1-3 pg/ml. The assay required about four hours of bench time to complete.

Materials: the following materials were used in the procedure described below: Assay plate: Nunc Maxisorp, product 464718, 384 well, clear, passively coated with a monoclonal antibody, BiosPacific #8311 (1 mg/ml in citrate-phosphate-NaC buffer pH 6.0, with 0.1% sodium azide as a preservative, stored at +2 to +8° C. For the standard curve, human PSA antigen (BioSpecific #J63000190) was used. The diluent for the standard concentrations was human serum. The standard was diluted to 10 ug/ml, aliquoted and frozen to −80° C. Dilution of the standards was done in a 96 well, conical, polypropylene, (Nunc product #249944). The following buffers and solutions were used: (a) assay buffer: BBS with 1% BSA and 0.1% Triton X-100; (b) detection antibody (Ab): goat polyclonal antibody affinity purified (BioSpecific G126C), which was labeled with fluorescent dye AlexaFluor 647, and stored at 4° C.; detection antibody diluent: 50% assay buffer, 50% passive blocking solution; wash buffer: borate buffer saline (BBS) with Triton X-100 (1.0 M borate, 15.0 M sodium chloride, 10% Triton X-100, pH=8.3); elution buffer: BBS with 4M urea, 0.02% Triton X-100 and 0.001% BSA.

Preparation of AlexaFluor 647 labeled antibodies: the detection antibody G-129-C was conjugated to AlexaFluor 647 by first dissolving 100 ug of G-129-C in 400 uL of the coupling buffer (0.1M NaHCO3). The antibody solution was then concentrated to 50 ul by transferring the solution into YM-30 filter and subjecting the solution and filter to centrifugation. The YM-30 filter and antibody was then washed three times by adding 400 ul of the coupling buffer. The antibody was recovered by adding 50 □l to the filter, inverting the filter, and centrifuging for 1 minute at 5,000×g. The resulting antibody solution was 1-2 ug/ul. AlexaFluor 647 NHS ester was reconstituted by adding 20 ul DMSO to one vial of AlexaFluor 647, this solution was stored at −20° C. for up to one month. 3 ul of AlexaFluor 647 stock solution was added to the antibody solution, which was then mixed and incubated in the dark for one hour. After the one hour, 7.5 ul 1M tris was added to the antibody AlexaFluor 647 solution and mixed. The solution was ultrafiltered with YM-30 to remove low molecular weight components. The volume of the retentate, which contained the antibody conjugated to AlexaFluor 647, was adjusted to 200-400 □l by adding PBS. 3 ul 10% NaN3 was added to the solution, the resulting solution was transferred to an Ultrafree 0.22 centrifugal unit and spun for 2 minutes at 12,000×g. The filtrate containing the conjugated antibody was collected and used in the assays.

Procedure: total PSA standard and sample preparation and analysis:

The standard curve was prepared as follows: working standards were prepared (0-120 pg/ml) by serial dilutions of the stock of PSA into standard diluent or to achieve a range of PSA concentrations of between 1.2 pg/ml-4.3 μg/ml.

10 μl passive blocking solution and 10 μl of standard or of sample were added to each well. Standards were run in quadruplicate. The plate was sealed with Axyseal sealing film, centrifuged for 1 min at 3000 RPM, and incubated for 2 hours at 25° C. with shaking. The plate was washed five times, and centrifuged until rotor reached 3000 RPM in an inverted position over a paper towel. A 1 nM working dilution of detection antibody was prepared, and 20 μl detection antibody were added to each well. The plate was sealed and centrifuged, and the assay incubated for 1 hour at 25° C. with shaking. 30 μl elution buffer were added per well, the plate was sealed and the assay incubated for 1 hour at 25° C. The plate was either stored for up to 48 hours at 4° C. prior to analysis, or the sample was analyzed immediately.

For analysis, 20 μl per well were acquired at 40 μl/minute, and 5 μl were analyzed at 5 μl/minute. The data were analyzed based on a threshold of 4 sigma. Raw signal versus concentration of the standards was plotted. A linear fit was performed for the low concentration range, and a non-linear fit was performed for the full standard curve. The limit of detection (LoD) was calculated as LOD=(3× standard deviation of zeros)/slope of linear fit. The concentrations of the samples were determined from the equation (linear or non-linear) appropriate for the sample signal.

An aliquot was pumped into the analyzer. Individually-labeled antibodies were measured during capillary flow by setting the interrogation volume such that the emission of only 1 fluorescent label was detected in a defined space following laser excitation. With each signal representing a digital event, this configuration enables extremely high analytical sensitivities. Total fluorescent signal is determined as a sum of the individual digital events. Each molecule counted is a positive data point with hundreds to thousands of DMC events/sample. The limit of detection of the total PSA assay of the invention was determined by the mean+3 SD method.

Results: Data for the low end of a typical PSA standard curve measured using the assay protocol is shown in Table 4. The limit of detection (LoD) of the four standards tested was less than 0.05 pg/ml.

TABLE 4
PSA Standard Curve - Low End Level of Detection <0.05 pg/ml

| | Detected Events - | | Limit of Detection |
|---|---|---|---|
| | Background (DE) | Slope | (LoD) |
| Standard 1 | 105 | 687 | 0.026 |
| Standard 2 | 105 | 747 | 0.061 |
| Standard 3 | 83 | 622 | 0.030 |
| Standard 4 | 97 | 721 | 0.032 |

The sensitivity of the analyzer was tested using seven precision curves generated on different days using different reagents as shown in Table 5. Linearized standard curve for the range of LoD are shown in FIG. 4.

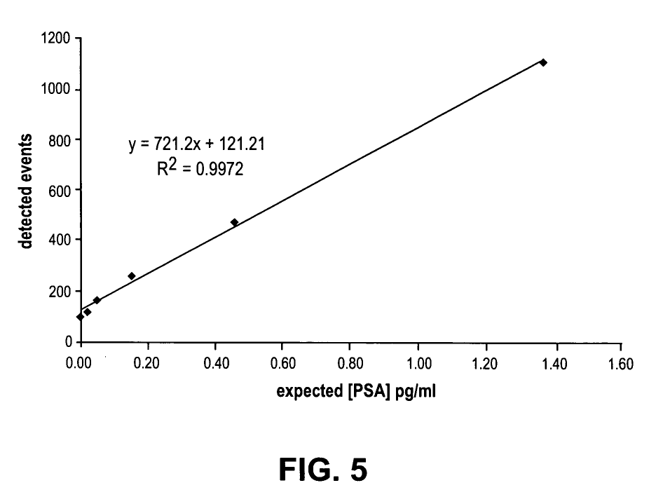

Linearized standard curves for the seven PSA samples are shown in FIG. 5. The seven curves are pack interpolated to show how well they fit together. The analytical limit of detection (LoD) was determined across seven different assays, each assay was generated on different days and consisted of different reagents. The average LoD ranged from 0.02 pg/ml to 108 pg/ml.

These data show that the analyzer system of the invention allows for performing highly sensitive laser induced immunoassay for sub-femtomolar concentrations of total PSA.

TABLE 5
Precision Curves of Seven Different Standard Curves

| Expected | cv 1 | cv 2 | cv 3 | cv 4 | cv 5 | cv 6 | cv 7 | avg | | |
|---|---|---|---|---|---|---|---|---|---|---|
| pg/ml | pg/ml | pg/ml | pg/ml | pg/ml | pg/ml | pg/ml | pg/ml | pg/ml | stdev | CV |
| 111 | 100 | 108 | 117 | 104 | 100 | 105 | 124 | 108 | 9 | 8% |
| 37 | 41 | 40 | 42 | 39 | 35 | 37 | 38 | 39 | 2 | 6% |
| 12 | 13 | 12 | 13 | 12 | 12 | 12 | 12 | 12 | 0 | 3% |
| 4.1 | 4.7 | 4.0 | 4.6 | 4.4 | 4.2 | 4.1 | 3.8 | 4.3 | 0.3 | 8% |
| 1.37 | 1.12 | 1.26 | 1.19 | 1.27 | 1.38 | 1.41 | 1.40 | 1.29 | 0.11 | 9% |
| 0.46 | 0.44 | 0.46 | 0.43 | 0.39 | 0.52 | 0.54 | 0.55 | 0.48 | 0.06 | 13% |
| 0.15 | 0.17 | 0.15 | 0.15 | 0.15 | 0.13 | 0.14 | 0.15 | 0.15 | 0.01 | 7% |
| 0.05 | 0.06 | 0.07 | 0.06 | 0.06 | 0.08 | 0.00 | 0.16 | 0.15 | 0.01 | 7% |
| 0.02 | 0.02 | 0.00 | 0.04 | 0.02 | 0.00 | 0.00 | 0.09 | 0.02 | 0.03 | 138% |

### Example 3

### Sandwich Bead-Based Assays for Total PSA

Assay: The assays described above use the same microtiter plate format where the plastic surface is used to immobilize target molecules. The single particle analyzer system also is compatible with assays done in solution using microparticles or beads to achieve separation of bound from unbound entities.

Materials: MyOne Streptavidin C1 microparticles (MPs) are obtained from Dynal (650.01-03, 10 mg/ml stock). Buffers used in the assay include: 10× borate buffer saline Triton Buffer (BBST) (1.0 M borate, 15.0 M sodium chloride, 10% Triton X-100, pH 8.3); assay buffer (2 mg/ml normal goat IgG, 2 mg/ml normal mouse IgG, and 0.2 mg/ml MAB-33-IgG-Polymer in 0.1 M Tris (pH 8.1), 0.025 M EDTA, 0.15 M NaCl, 0.1% BSA, 0.1% Triton X-100, and 0.1% NaN3, stored at 4C); sand elution buffer (BBS with 4 M urea, 0.02% Triton X-100, and 0.001% BSA, stored at 2-8C). Antibodies used in the sandwich bead-based assay include: Bio-Ab (A34650228P (BiosPacific) with 1-2 biotins per IgG) and Det-Ab (G-129-C (BiosPacific) conjugated to A647, 2-4 fluors per IgG). The standard is anti-human PSA (BiosPacific, cat #J63000190). The calibrator diluent is 30 mg/ml BSA in TBS wEDTA.

Microparticles Coating: 100 ul of the MPs stock is placed in an eppendorf tube. The MPs are washed three times with 100 ul of BBST wash buffer by applying a magnet, removing the supernatant, removing the magnet, and resuspending in wash buffer. After the washes the MPs are resuspended in 100 ul of assay buffer and 15 ug of Bio-Ab are added. The mixture is then incubated for 30 minutes at room temperature (25° C.) with constant shaking. The MPs are washed five times with 1 ml wash buffer as described above. After the washes the MPs are resuspended in 15 ml of assay buffer (or 100 ul to store at 4° C.).

Preparation of Standard and Samples: the Standard is Diluted with Calibrator Diluent to Prepare Proper Standard curve (usually 200 pg/ml down to 0.1 pg/ml). Frozen serum and plasma samples need to be centrifuged 10 minutes at room temperature at 13 K rpm. Clarified serum/plasma is removed carefully to avoid taking any possible pellets or floaters and put into fresh tubes. 50 ul of each standard or sample is pipetted into appropriate wells.

Capture Target: 150 ul of MPs (after resuspension to 15 ml in assay buffer+400 mM NaCl) are added to each well. The mixture is incubated on JitterBug, 5 at room temperature for 1 hr.

Washes and Detection: The plate is placed on a magnet and the supernatant is removed after ensuring that all MPs are captured by the magnet. 250 ul of wash buffer are added after removing the plate from the magnet. The plate is then placed on the magnet and the supernatant is removed after ensuring that all MPs are captured by the magnet. 20 ul Det-Ab are added per well (Det-Ab to 500 ng/ml is diluted in assay buffer+400 mM NaCl)). The mixture is incubated on JitterBug, 5 at room temperature for 30 min.

Washes and Elution: The plate is placed on a magnet and washed three times with wash buffer. The supernatant is removed after ensuring that all MPs are captured by the magnet and 250 ul of wash buffer are added. After the washes the samples are transferred into a new 96-well plate. The new plate is then placed on the magnet and the supernatant is removed after ensuring that all MPs are captured by the magnet. 250 ul of wash buffer are then added after removing the plate from the magnet. The plate is then placed on the magnet and the supernatant is removed after ensuring that all MPs are captured by the magnet. 20 ul of elution buffer are then added and the mixture is incubated on JitterBug, 5 at room temperature for 30 min.

Filter out MPs and transfer to 384-well plate: The standard and samples are transferred into a 384-well filter plate placed on top of a 384-well assay plate. The plate is then centrifuged at room temperature at 3000 rpm with a plate rotor. The filter plate is removed and the appropriate calibrators are added. The plate is covered and is ready to be run on SMD.

Single Molecule Detector: An aliquot is pumped into the analyzer. Individually-labeled antibodies are measured during capillary flow by setting the interrogation volume such that the emission of only 1 fluorescent molecule is detected in a defined space following laser excitation. With each signal representing a digital event, this configuration enables extremely high analytical sensitivities. Total fluorescent signal is determined as a sum of the individual digital events. Each molecule counted is a positive data point with hundreds to thousands of DMC events/sample. The limit of detection the total PSA assay of the invention is determined by the mean+3 SD method.

### Example 4

### Concentration Range for Total PSA in Male Populations

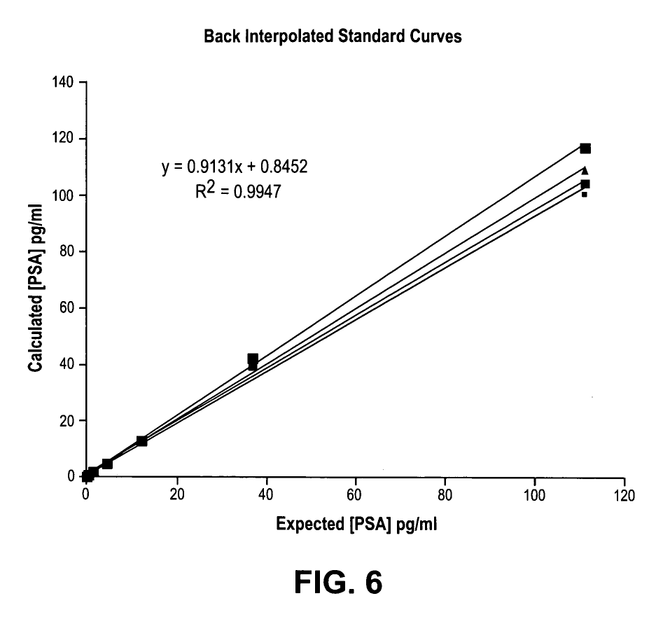

Male plasma samples were measured for PSA using the sandwich immunoassay as described in Example 1 above and the number of signals or events as described above were counted using the single particle analyzer system of the invention. The concentration of serum PSA was determined by correlating the signals detected by the analyzer with the standard curves as described above. Male plasma samples from male patients were diluted to 1:100, 1:1000, and 1:10,000. The plasma samples from four males were measure for PSA levels. The samples were then diluted to 1:100, 1:1000, and 1:10,000. The estimated concentration of PSA corresponding to the level of dilution was plotted on the graph shown in FIG. 6. The graph shows the linearity of the dilution series.

### Example 5

### Concentration Range for Total PSA in a Female Population of Total Non-Diseased Subjects

A reference range or normal range for total PSA concentrations in human serum was established using serum and plasma samples from 53 apparently healthy subjects (non-diseased). A sandwich immunoassay as described in Example 1 was performed and the number of signals or events as described above were counted using the single particle analyzer system of the invention. The concentration of serum PSA was determined by correlating the signals detected by the analyzer with the standard curve as described above. All assays were preformed once on each individual.

PSA in women may be an indicator of breast cancer, or the development of breast cancer. Current methods for PSA testing allow for the detection of PSA levels down to 5-10 pg/ml and is as low as 3 pg/ml. The range of PSA levels in normal women has not yet been determined.

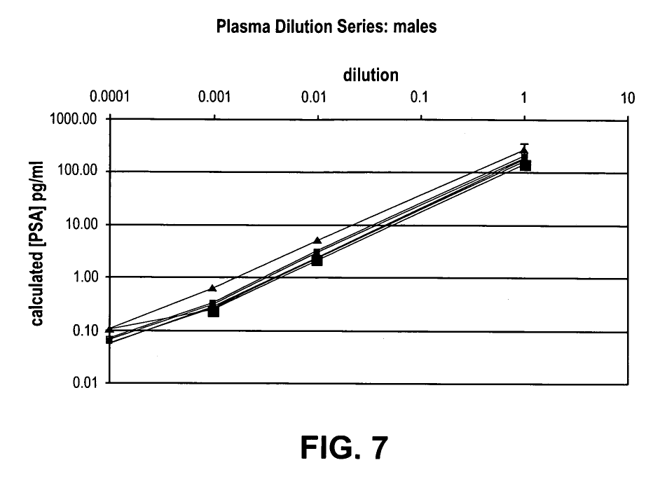

The assay of the invention is sufficiently sensitive and precise to determine levels of PSA as low as less than approximately 1 pg/ml, less than approximately 0.5 pg/ml, and less than approximately 0.1 pg/ml. The assay of the invention is sufficiently sensitive and precise to measure PSA levels in women as low as 0.1, as seen in FIGS. 7A and 7B.

### Example 6

### Erenna Magnetic Particle (MP)-Based Immunoassay for Detection of Free and Total PSA in Normal Female Serum

Recent advances in immunological detection of prostate specific antigen (PSA) in females have shown it to have promise as a potential biomarker for diagnosis and staging of breast cancer. As in prostate cancer, the relative proportion of free to total PSA is elevated in breast cancer patients, and has been shown to decrease after surgery. However, the diagnostic utility of free PSA for breast cancer diagnosis is currently limited by the poor sensitivity of available assays. A novel assay system, the Erenna™ Immunoassay (Singulex) utilizing single molecule counting and paramagnetic microparticles (MPs) has been shown to have increased sensitivity and precision, which could potentially be applied to increase the sensitivity of PSA quantification in female patients. The sensitivity of the Erenna MP-based Immunoassay for quantification of free and total PSA concentration in serum obtained from healthy female subjects. Thus in some embodiments of the invention, free PSA, total PSA, or the ratio of free PSA to total PSA is used to determine a diagnosis, prognosis, or choice of treatment in breast cancer patients or individuals suspected of having breast cancer.

Sample preparation: Serum samples from 48 normal female blood donors between the ages of 18 and 57 years were obtained. Donors were free of apparent malignancies at the time of collection. Concentrations of free PSA and total PSA were assayed using 50 ul of serum per sample. For each MP-based assay, analytes were either detected with total PSA (Diagnostic Systems Laboratories, Inc.) or free PSA (BiosPacific) capture and detection antibody sets.

Procedure: Assays were carried out with a 60 minute capture, a 30 minute capture, and a 45 minute elution step. Determinations were made in triplicate as an average (+/−std dev). Ratios of free PSA to total PSA were calculated and expressed as percentages of free PSA. Preliminary assay sensitivity, limit of quantification and precision was determined by back interpolation of standard curves over 6 assay runs.

Results: Average analytical sensitivity of the Erenna free and total PSA assays were 0.031 ng/L and 0.023 ng/L, respectively. Precision ranged from 3-8% and 2-12% CV (0.1-25 pg/mL) for the Erenna free and total PSA assays, respectively. Total PSA was measurable in all donors and free PSA was measurable in 93.75% (45/48) of donors. Concentration of free PSA varied in normal female sera from 0.07-5.41 pg/mL, compared to concentrations from 0.24-49.12 pg/mL for total PSA. No trends were observed in concentration of free or total PSA with respect to age. Ratios of free to total PSA ranged from 1% to 88% free PSA, with a median ratio of 16% free PSA and with 8.3% (4/48) of samples having a ratio>50%. The Erenna PSA Immunoassay was able to quantify free and total PSA in all sera obtained from healthy female subjects. The increased sensitivity and precision of the Erenna PSA assay will allow for future studies evaluating free PSA and total PSA as a potential diagnostic biomarker for early detection of breast cancer.

What is claimed is:

1. A method for detecting a single prostate specific antigen (PSA) molecule, fragment, or complex in a sample at a level less that 100 pg/ml comprising:
a. labeling said PSA molecule, fragment, or complex, if present, with a label; and
b. detecting the presence or absence of said label, wherein detection of the presence of said label indicates the presence of said single PSA molecule, fragment, or complex of PSA in said sample.

2. The method of claim 1, wherein the level of PSA detected is less than 5 pg/ml.

3. The method of claim 1, wherein the level of PSA detected is less than 1 pg/ml.

4. The method of claim 1, wherein the level of PSA detected is less than 0.1 pg/ml.

5. The method of claim 1, wherein the PSA is selected from total PSA, free PSA, a PSA complex, PSA-ACT, and PSA-A2M.

6. The method of claim 1, wherein said detecting is capable of detecting said single molecules of PSA at a limit of detection of less than 100 pg/ml.

7. The method of claim 1, wherein said label comprises a fluorescent moiety.

8. The method of claim 7, wherein said fluorescent moiety comprises a dye selected from the group consisting of AlexaFluor 488, AlexaFluor 532, AlexaFluor 647, AlexaFluor 680, or AlexaFluor 700.

9. The method of claim 1, wherein said label further comprises a binding partner for said PSA molecule, fragment, or complex.

10. The method of claim 9, wherein said binding partner comprises an antibody specific to said PSA molecule, fragment, or complex.

11. The method of claim 1, further comprising capturing said PSA or PSA complex on a solid support.

12. The method of claim 11, wherein said solid support comprises a capture partner specific for said PSA or PSA complex that is attached to said solid support.

13. The method of claim 12, wherein said attachment of said capture partner to said solid support is selected from a noncovalent or covalent bond.

14. The method of claim 12, wherein said capture partner comprises an antibody.

15. The method of claim 1, wherein said label comprises a fluorescent moiety, and wherein step (b) comprises passing said label through a single-molecule detector.

16. The method of claim 15, wherein said single molecule detector comprises
a. an electromagnetic radiation source for stimulating said fluorescent moiety;
b. a capillary flow cell for passing said fluorescent moiety;
c. a source of motive force for moving said fluorescent moiety in said capillary flow cell;
d. an interrogation space defined within said capillary flow cell for receiving electromagnetic radiation emitted from said electromagnetic source;
e. an electromagnetic radiation detector operably connected to said interrogation space for measuring an electromagnetic characteristic of said stimulated fluorescent moiety; and
f. a microscope objective lens situated between said interrogation space and said detector, wherein the lens has a numerical aperture of 0.6 or greater.

17. A method of diagnosing prostate cancer based on levels of PSA wherein the level of PSA in a sample is detected at a level of sensitivity of less than 100 pg/ml.

18. The method of claim 17 wherein the diagnosis is of a recurrence of prostate cancer after surgical resection.

19. The method of claim 17 wherein the diagnosis of recurrence of prostate cancer is made by measuring the PSA levels from a series of samples.

20. The method of claim 17 wherein the diagnosis of recurrence of prostate cancer is made by measuring the change in levels of PSA in a series of samples.

21. A method for detecting a single prostate specific antigen (PSA) molecule, fragment, or complex in a sample at a level less that 5 pg/ml comprising:
a. labeling said PSA molecule, fragment, or complex, if present, with a label; and
b. detecting the presence or absence of said label, wherein detection of the presence of said label indicates the presence of said single PSA molecule, fragment, or complex of PSA in said sample.

22. The method of claim 21 wherein the level of PSA detected is in the range of about 0.1 pg/ml to about 1 pg/ml.

23. A method for assessing the likelihood of recurrence of cancer in an individual comprising:
a. determining a concentration of prostate specific antigen in a sample or determining the concentrations of prostate specific antigen in a series of samples from said individual, wherein the concentration is determined by a prostate specific antigen assay with a limit of detection of said prostate specific antigen in a sample less than about 5 pg/ml; and
b. determining the likelihood of recurrence of cancer for said individual, based on the concentration of prostate specific antigen in said sample, or in said concentrations of prostate specific antigen in said series of samples.

24. The method of claim 23 wherein step (b) further comprises measuring a change in concentration of prostate specific antigen in a sequential series of samples from said individual whereby said change is used to assess the likelihood or recurrence of cancer in said individual.

25. A method of monitoring decreases in a level of PSA after surgical resection comprising measuring the level of PSA in a first sample from an individual wherein said first sample is taken from said individual prior to surgical resection and further measuring the level of PSA in a second sample, said second sample taken from said individual after surgical resection is complete, and further comparing the levels of PSA in said first and second samples.

26. The method of claim 25 further comprising measuring the level of PSA in a series of samples taken from said individual after surgical resection and comparing the level of PSA from each sample from said series of sample to the level of PSA in said sample taken from said first sample.

27. A method of monitoring the effectiveness of a therapeutic treatment in an individual comprising measuring the level of PSA in a first sample from said individual wherein said first sample is taken prior to administration of said therapeutic treatment and further comprising measuring the level of PSA in a series of samples taken from said individual at different time points subsequent to beginning said therapeutic treatment and further comparing the level of PSA prior to said therapeutic treatment to the level of PSA subsequent to said therapeutic treatment to determine the effectiveness of said therapeutic treatment.

28. The method of claim 27 wherein said therapeutic treatment is altered in response to the level of PSA measured subsequent to said therapeutic treatment.

29. A method for diagnosing breast cancer in an individual comprising comparing a level of PSA in a sample obtained from said individual to a normal range of PSA levels wherein the normal range is determined from a distribution of PSA levels in a reference population consisting of normal individuals.

30. The method of claim 29 further comprising detecting levels of PSA in a sample at a sensitivity of less than 100 pg/ml.

31. A method for screening an individual for the presence of breast cancer comprising detecting a level of PSA in a series of samples taken from said individual and further detecting increases or decreases in PSA levels of said individual compared to a predetermined threshold level of PSA.

32. The method of claim 31 wherein PSA levels in the series of samples are detected at a level of sensitivity of less than 100 pg/ml.

33. A method of diagnosing a condition indicated by increased levels of kallikreins wherein the level of kallikreins in a sample is detected at a level of sensitivity of less than 100 pg/ml.

34. The method of claim 33 wherein the kallikrein detected is human killikrein 3 (hk3).

35. A method for determining a diagnosis, prognosis, or method of treatment in an individual comprising:
a. determining a concentration of prostate specific antigen in a sample or determining the concentrations of prostate specific antigen in a series of samples from said individual, wherein said concentration is determined by a prostate specific antigen assay with a limit of detection for said prostate specific antigen in a sample less than about 100 pg/ml; and
b. determining a diagnosis, prognosis, or method of treatment for said individual, based on said concentration of PSA in said sample, or on said concentrations of PSA in said series of samples.

36. The method of claim 35 wherein step (b) comprises a diagnosis, prognosis or method of treatment for an individual in which recurrence of prostate cancer has occurred.

37. The method of claim 35 wherein step (b) comprises a diagnosis, prognosis, or method of treatment for an individual having or susceptible to having breast cancer.

38. The method of claim 35 wherein step (b) comprises an analysis selected from the group consisting of comparing said concentration or series of concentrations to a normal value for said concentration, comparing said concentration or series of concentrations to a baseline value, and determining a rate of change of concentration for said series of concentrations.

39. The method of claim 35 wherein step (b) comprises comparing said concentration of PSA in a said sample with a predetermined threshold concentration, and determining a diagnosis, prognosis, or method of treatment if the sample concentration is greater than the threshold level.

40. A composition for the detection of a prostate specific antigen (PSA) molecule, fragment, or complex comprising a binding partner to the prostate specific antigen molecule, fragment, or complex attached to a fluorescent moiety, wherein said fluorescent moiety is capable of emitting at least about 200 photons when stimulated by a laser emitting light at the excitation wavelength of the moiety, wherein the laser is focused on a spot not less than about 5 microns in diameter that contains the moiety, and wherein the total energy directed at the spot by the laser is no more than about 3 microJoules.

41. The composition of claim 40 wherein said binding partner comprises an antibody to said PSA molecule, fragment, or complex.

42. The composition of claim 40 wherein said PSA is selected from free PSA, total PSA, or a PSA complex.

43. The composition of claim 42 wherein the PSA complex is selected from PSA-ACT and PSA-A2M.

44. The composition of claim 40 wherein said fluorescent moiety comprises a molecule that comprises at least one substituted indolium ring system in which the substituent on the 3-carbon of the indolium ring contains a chemically reactive group or a conjugated group.

45. The composition of claim 40 wherein said fluorescent moiety comprises a dye selected from the group consisting of AlexaFluor 488, AlexaFluor 532, AlexaFluor 647, AlexaFluor 680, AlexaFluor 700.

46. A composition comprising a set of standards for the determination of a concentration of a PSA wherein at least one of the standards is at a concentration of PSA less than about 100 pg/ml.

47. A kit comprising a composition comprising an antibody to prostate specific antigen attached to a fluorescent dye moiety, wherein said moiety is capable of emitting at least about 200 photons when stimulated by a laser emitting light at the excitation wavelength of the moiety, wherein the laser is focused on a spot not less than about 5 microns in diameter that contains the moiety, and wherein the total energy directed at the spot by the laser is no more than about 3 microJoules, wherein said composition is packaged in suitable packaging.

48. The kit of claim 47 wherein the PSA is free PSA, total PSA, or a PSA complex.

49. The kit of claim 48 wherein the complexed PSA is selected from PSA-ACT or PSA-A2M.

50. The kit of claim 47 further comprising a composition comprising a capture antibody attached to a solid support.

51. The kit of claim 47 further comprising a composition comprising a capture antibody for complexed PSA attached to a solid support.

52. The kit of claim 51 wherein said solid support comprises a microtiter plate or paramagnetic microparticles.

53. The kit of claim 47 further comprising a component selected from the group consisting of wash buffer, assay buffer, elution buffer, and calibrator diluent.

54. The kit of claim 47 further comprising a standard for the PSA.
